(19) United States
(12) Patent Application Publication
(10) Pub. No.: US 2021/0198167 A1
(43) Pub. Date: Jul. 1, 2021

(54) ORGANIC ELECTROLUMINESCENT COMPOUND AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Doo-Hyeon Moon, Gyeonggi-do (KR); Dong-Hyung Lee, Gyeonggi-do (KR); Du-Yong Park, Gyeonggi-do (KR)

(21) Appl. No.: 17/119,726

(22) Filed: Dec. 11, 2020

(30) Foreign Application Priority Data
Dec. 26, 2019 (KR) 10-2019-0174692
Oct. 23, 2020 (KR) 10-2020-0138233

(51) Int. Cl. C07C 13/66; C09K 11/06; H01L 51/00

(52) U.S. Cl. CPC C07C 13/66; C09K 11/06; H01L 51/0054; H01L 51/006; H01L 51/0072; H01L 51/5056; H01L 51/0074; C09K 2211/1011; C09K 2211/1029; C09K 2211/1088; C09K 2211/1092; H01L 51/0073

(57) ABSTRACT

The present disclosure relates to an organic electroluminescent compound and an organic electroluminescent device comprising the same. The organic electroluminescent device having improved driving voltage and/or luminous efficiency can be provided by using the organic electroluminescent compound according to the present disclosure.

### TECHNICAL FIELD

The present disclosure relates to an organic electroluminescent compound and an organic electroluminescent device comprising the same.

### BACKGROUND ART

In 1987, Tang et al. of Eastman Kodak first developed a small molecule green organic electroluminescent device (OLED) of TPD/Alq₃ bilayer consisting of a light-emitting layer and a charge transport layer. Thereafter, the development of OLEDs was rapidly effected and OLEDs have been commercialized. An OLED changes electric energy into light by applying electricity to an organic light-emitting material, and commonly comprises an anode, a cathode, and an organic layer between the two electrodes.

The organic layer of the organic electroluminescent device may comprise a hole injection layer, a hole transport layer, a hole auxiliary layer, a light-emitting auxiliary layer, an electron blocking layer, a light-emitting layer, an electron buffer layer, a hole blocking layer, an electron transport layer, an electron injection layer, etc. The materials used in the organic layer can be classified into a hole injection material, a hole transport material, a hole auxiliary material, a light-emitting auxiliary material, an electron blocking material, a light-emitting material (including host and dopant materials), an electron buffer material, a hole blocking material, an electron transport material, an electron injection material, etc., depending on their functions.

Recently, attempts have been made to improve color purity and/or light extraction efficiency by optimizing an optical thickness between an anode and a cathode in order to improve the characteristics of an organic electroluminescent device. For example, the thickness of the organic layer and the refractive index of the organic layer included in the device may be adjusted in order to satisfy the efficient light extraction condition of a specific wavelength for implementing a specific color, and the two are in a complementary relationship with each other. That is, in order to achieve specific optical properties, if a material having high refractive index is used, the thickness of the device can be reduced. This makes it possible to make the device thinner and to reduce the amount of material used. Accordingly, there is a need for development of an organic electroluminescent material having high refractive index properties. Conventionally, it has already been studied how certain materials exhibit high refractive indices (J. phys. Chem. A. 1999, 103, 1818 to 1821). However, most papers and patents only disclosed the comparison results in similar structures, and do not accurately match with the tendency in diverse structures (J. Mater. Chem., 2011, 21, 19187-19202).

In addition, Korean Patent Appln. Laid-Open No. 2017-0124957 discloses a compound in which a diarylamine is bonded to 5-position of benzo[b]fluorene, but an organic electroluminescent compound having a higher refractive index is still required.

### DISCLOSURE OF INVENTION

### Technical Problems

The objective of the present disclosure is at least one of the following: The present disclosure provides an organic electroluminescent compound having a novel structure. The present disclosure provides a hole transport material comprising an organic electroluminescent compound having a novel structure. The present disclosure provides an organic electroluminescent compound having a higher refractive index compared to a conventional organic electroluminescent compound.

### Solution to Problem

The present inventors found that a compound substituted with an amine group at a specific position of benzo[b]fluorene has effective properties as an organic electroluminescent compound and can also provide a high refractive index. Specifically, the objective can be achieved by an organic electroluminescent compound represented by the following formula 1.

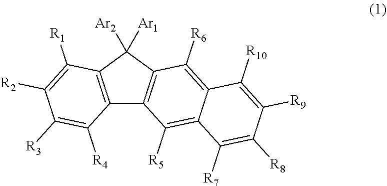

wherein

Ar₁ and Ar₂ each independently represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, or a substituted or unsubstituted (3- to 30-membered)heteroaryl, or Ar₁ and Ar₂ may be linked to each other to form a fused ring;

R₁ to R₆ each independently represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsiyl, or a substituted or unsubstituted tri(C6-C30)arylsiyl;

R₇ to R₁₀ each independently represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arysilyl, a substituted or unsubstituted (C1-C30)alkyldi(C6-C30)arylsilyl, a substituted or unsubstituted tri(C6-C30)arylsilyl, or

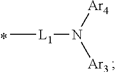

with a proviso that at least one of R₇ to R₁₀ is

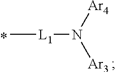

L₁ is a single bond, a substituted or unsubstituted (C6-C30)arylene, or a substituted or unsubstituted (3- to 30-membered)heteroarylene;

Ar₃ and Ar₄ each independently represent hydrogen, deuterium, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, or a substituted or unsubstituted (3- to 30-membered)heteroaryl, or Ara and Ar₄ may be linked to each other to form a fused ring; and

* represents the position linked to benzofluorene.

### Advantageous Effects of Invention

The present disclosure provides at least one of the following advantages: According to the present disclosure, it is possible to obtain an organic electroluminescent compound, preferably a hole transport material, having a novel structure that can be used in an organic electroluminescent device. The organic electroluminescent compound according to the present disclosure has a higher refractive index than conventional compounds having similar structures. By using the organic electroluminescent compound according to the present disclosure, it is possible to manufacture a thinner organic electroluminescent device and reduce the amount of materials used. By using the organic electroluminescent compound according to the present disclosure, it is possible to obtain an organic electroluminescent device with improved driving voltage and/or luminous efficiency.

### MODE FOR THE INVENTION

Hereinafter, the present disclosure will be described in detail. However, the following description is intended to explain the invention, and is not meant to restrict the scope of the invention.

The term “organic electroluminescent compound” in the present disclosure means a compound that may be used in an organic electroluminescent device, and may be comprised in any layer constituting an organic electroluminescent device, as necessary.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material, an electron buffer material, a hole blocking material, an electron transport material, or an electron injection material.

Herein, the term “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. The term “(C2-C30)alkenyl” is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. The term “(C2-C30)alkynyl” is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl, etc. The term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. The term “(3- to 7-membered)heterocycloalkyl” is meant to be a cycloalkyl having 3 to 7, preferably 5 to 7 ring backbone atoms, and including at least one heteroatom selected from the group consisting of B, N, O, S, Si, and P, preferably at least one heteroatom selected from the group consisting of O, S, and N. The above heterocycloalkyl may include tetrahydrofuran, pyrrolidine, thiolane, tetrahydropyran, etc. The term “(C6-C30)aryl(ene)” is meant to be a monocyclic or fused ring radical derived from an aromatic hydrocarbon having 6 to 30 ring backbone carbon atoms, which may be partially saturated. The number of ring backbone carbon atoms is preferably 6 to 25, and more preferably 6 to 18. The above aryl comprises those having a spiro structure. The above aryl may include phenyl, biphenyl, terphenyl, naphthyl, binaphthyl, phenylnaphthyl, naphthylphenyl, phenylterphenyl, fluorenyl, phenylfluorenyl, diphenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, azulenyl, tetramethyldihydrophenanthrenyl, etc. More specifically, the aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenylyl, 3-biphenyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenylyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl(ene)” means an aryl group having 3 to 30 ring backbone atoms and including at least one heteroatom(s) selected from the group consisting of B, N, O, S, Si, and P. The number of heteroatoms is preferably 1 to 4. The above heteroaryl(ene) may be a monocyclic ring or a fused ring condensed with at least one benzene ring, and may be partially saturated. In addition, the above heteroaryl(ene) comprises the form in which at least one heteroaryl or aryl group is linked to a heteroaryl group via a single bond(s), and also comprises those having a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazoyl, triazinyl, tetrazinyl, triazolyl, tetrazoyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, dibenzoselenophenyl, naphthobenzofuranyl, naphthobenzothiophenyl, benzofuroquinoyl, benzofuroquinazolinyl, benzofuronaphthyridinyl, benzofuropyrimidinyl, naphthofuropyrimidinyl, benzothienoquinoyl, benzothienoquinazolinyl, benzothienonaphthyridinyl, benzothienopyrimidinyl, naphthothienopyrimidinyl, pyrimidoindolyl, benzopyrimidoindoyl, benzofuropyrazinyl, naphthofuropyrazinyl, benzothienopyrazinyl, naphthothienopyrazinyl, pyrazinoindolyl, benzopyrazinoindolyl, benzimidazolyl, benzothiazolyl, benzoisothiazolyl, benzoisoxazolyl, benzoxazolyl, isoindolyl, indolyl, benzoindoyl, indazolyl, benzothiadiazoyl, quinolyl, isoquinolyl, cinnolinyl, quinazolinyl, benzoquinazolinyl, quinoxalinyl, benzoquinoxalinyl, naphthyridinyl, carbazolyl, benzocarbazolyl, dibenzocarbazolyl, phenoxazinyl, phenothiazinyl, phenanthridinyl, benzodioxolyl, dihydroacridinyl, benzotriazole phenazinyl, imidazopyridyl, chromenoquinazolinyl, thiochromenoquinazolinyl, dimethylbenzoperimidinyl, indoocarbazolyl, indenocarbazolyl, etc. More specifically, the heteroaryl may include 1-pyrroyl, 2-pyrroyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazoyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indoyl, 5-indoyl, 6-indoyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinoyl, 1-isoquinolyl, 3-isoquinoyl, 4-isoquinolyl, 5-isoquinolyl, 6-isoquinolyl, 7-isoquinolyl, 8-isoquinoyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazoyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazoyl, 4-oxazoyl, 5-oxazoyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. “Halogen” includes F, Cl, Br, and I.

In addition, “ortho (o-),” “meta (m-),” and “para (p-)” are prefixes, which represent the relative positions of substituents, respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

In addition, “substituted” in the expression “substituted or unsubstituted” means that a hydrogen atom in a certain functional group is replaced with another atom or another functional group (i.e., a substituent), and also comprises being substituted with a group in which two or more of the substituents are linked. For example, “a substituent in which two or more substituents are linked” may be pyridine-triazine. That is, pyridine-triazine may be interpreted as one heteroaryl substituent or the substituent in which two heteroaryl substituents are linked. In the present disclosure, the substituents of the substituted alkyl, the substituted aryl, the substituted arylene, the substituted heteroaryl, the substituted heteroarylene, the substituted cycloalkyl, the substituted heterocycloalkyl, the substituted trialkylsilyl, the substituted dialkylarylsilyl, the substituted alkyldiarylsilyl, and the substituted triarylsilyl, each independently, are at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with at least one of a (C1-C30)alkyl(s), a (3- to 30-membered)heteroaryl(s) and a di(C6-C30)arylamino(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C2-C30)alkenylamino; a mono- or di-(C6-C30)arylamino; a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3-to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituents, each independently, are at least one selected from the group consisting of a (C1-C20)alkyl; a (5- to 25-membered)heteroaryl unsubstituted or substituted with a (C6-C18)aryl(s); and a (C6-C25)aryl unsubstituted or substituted with at least one of a (C1-C30)alkyl(s) and a (5- to 25-membered)heteroaryl(s). According to another embodiment of the present disclosure, the substituents, each independently, are at least one selected from the group consisting of a (C1-C10)alkyl, a (5- to 25-membered)heteroaryl, and a (C6-C25)aryl. For example, the substituents, each independently, may be at least one selected from the group consisting of methyl, phenyl, and naphthyl.

In the formulas of the present disclosure, in case adjacent substituents are linked to each other to form a ring, the ring may be a substituted or unsubstituted mono- or polycyclic (3- to 30-membered) alicyclic or aromatic ring, or the combination thereof. In addition, the formed ring may contain at least one heteroatom selected from N, O, and S. The above ring may be preferably a substituted or unsubstituted mono- or polycyclic (3- to 26-membered) alicyclic or aromatic ring, or the combinations thereof, more preferably an unsubstituted mono- or polycyclic (5- to 15-membered) aromatic ring. In addition, the ring may form a spiro ring with a backbone structure. For example, the above ring may be a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, a substituted or unsubstituted carbazole ring, etc.

In the formulas of the present disclosure, the heteroaryl(ene) and the heterocycloalkyl, each independently, may contain at least one heteroatom selected from B, N, O, S, Si, and P. Also, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (5- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arysilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, and a substituted or unsubstituted (C1-C30)alkyl(C6-C30)arylamino.

Hereinafter, the compound represented by formula 1 will be described in more detail.

In formula 1, Ar₁ and Ar₂ each independently represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, or a substituted or unsubstituted (3- to 30-membered)heteroaryl, or Ar₁ and Ar₂ may be linked to each other to form a fused ring. Ar₁ and Ar₂ may be the same or different. According to one embodiment of the present disclosure, Ar₁ and Ar₂ each independently are a substituted or unsubstituted (C1-C20)alkyl or a substituted or unsubstituted (C6-C25)aryl, or Ar₁ and Ar₂ may be linked to each other to form a fused ring. According to another embodiment of the present disclosure, Ar₁ and Ar₂ each independently are an unsubstituted (C1-C10)alkyl or an unsubstituted (C6-C25)aryl, or Ar₁ and Ar₂ may be linked to each other to form a fused ring. For example, Ar₁ and Ar₂ may be each independently methyl or phenyl, or may be linked to each other to form a fluorene ring.

In formula 1, R₁ to R₆ each independently represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsiyl, or a substituted or unsubstituted tri(C6-C30)arylsilyl. According to one embodiment of the present disclosure, R₁ to R₆ each independently are hydrogen, deuterium, a substituted or unsubstituted (C6-C25)aryl, or a substituted or unsubstituted (5-to 25-membered)heteroaryl. According to another embodiment of the present disclosure, R to R₆ each independently are hydrogen or an unsubstituted (C6-C18)aryl. For example, R₁ to R₆ each independently may be hydrogen or a phenyl.

In formula 1, R₇ to R₁₀ each independently represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or

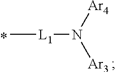

with a proviso that at least one of R₇ to R₁₀ is

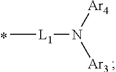

and * represents the position linked to benzofluorene. According to one embodiment of the present disclosure, R₇ to R₁₀ each independently are hydrogen, deuterium, a substituted or unsubstituted (C1-C10)alkyl, a substituted or unsubstituted (C6-C25)aryl, a substituted or unsubstituted (5- to 25-membered)heteroaryl, or

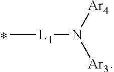

According to another embodiment of the present disclosure, any one of R₇ to R₁₀ is

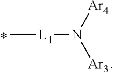

For example, any one of R₇ to R₁₀ may be

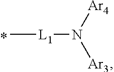

and the others may be hydrogen.

L₁ is a single bond, a substituted or unsubstituted (C6-C30)arylene, or a substituted or unsubstituted (3- to 30-membered)heteroarylene. According to one embodiment of the present disclosure, L₁ is a single bond or a substituted or unsubstituted (C6-C25)arylene. According to another embodiment of the present disclosure, L₁ is a single bond or a substituted or unsubstituted (C6-C18)arylene. For example, L₁ may be a single bond, phenylene, or naphthylene.

Ar₃ and Ar₄ each independently represent hydrogen, deuterium, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, or a substituted or unsubstituted (3- to 30-membered)heteroaryl, or Ar₃ and Ar₄ may be linked to each other to form a fused ring. Ar₃ and Ar₄ may be the same of different. According to one embodiment of the present disclosure, Ar₃ and Ar₄ each independently are a substituted or unsubstituted (C6-C25)aryl, or a substituted or unsubstituted (5- to 25-membered)heteroaryl. According to another embodiment of the present disclosure, Ar₃ and Ar₄ each independently are a (C6-C25)aryl unsubstituted or substituted with at least one (C1-C10)alkyl(s), or a (5- to 25-membered)heteroaryl unsubstituted or substituted with at least one (C6-C18)aryl(s). For example, Ar₃ and Ar₄ each independently may be phenyl, biphenyl, naphthylphenyl, phenylnaphthyl, terphenyl, dimethylfluorenyl, diphenylfluorenyl, 9,9′-spirobifluorenyl, dibenzothiophenyl, dibenzofuranyl, or carbazolyl substituted with a phenyl(s).

Formula 1 may be represented by at least one of the following formulae 1-1 to 1-4:

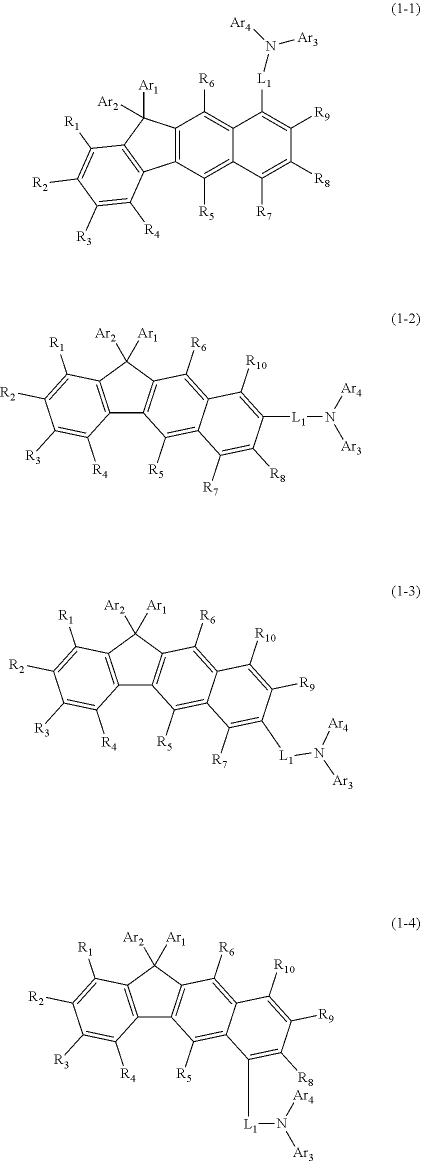

wherein

Ar₁ to Ar₄, R₁ to R₆, and L₁ are as defined in formula 1.

In formulae 1-1 to 1-4, R₇ to R₁₀ each independently represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arysilyl, or a substituted or unsubstituted tri(C6-C30)arylsilyl. According to one embodiment of the present disclosure, R₇ to R₁₀ each independently are hydrogen, deuterium, a substituted or unsubstituted (C1-C20)alkyl, a substituted or unsubstituted (C6-C25)aryl, or a substituted or unsubstituted (5- to 25-membered)heteroaryl. According to another embodiment of the present disclosure, R₇ to R₁₀ each independently are hydrogen, deuterium, an unsubstituted (C1-C10)alkyl, an unsubstituted (C6-C18)aryl, or a substituted or unsubstituted (5- to 20-membered)heteroaryl. For example, R₇ to R₁₀ may be hydrogen.

The compound represented by formula 1 may be at least one selected from the following compounds, but is not limited thereto.

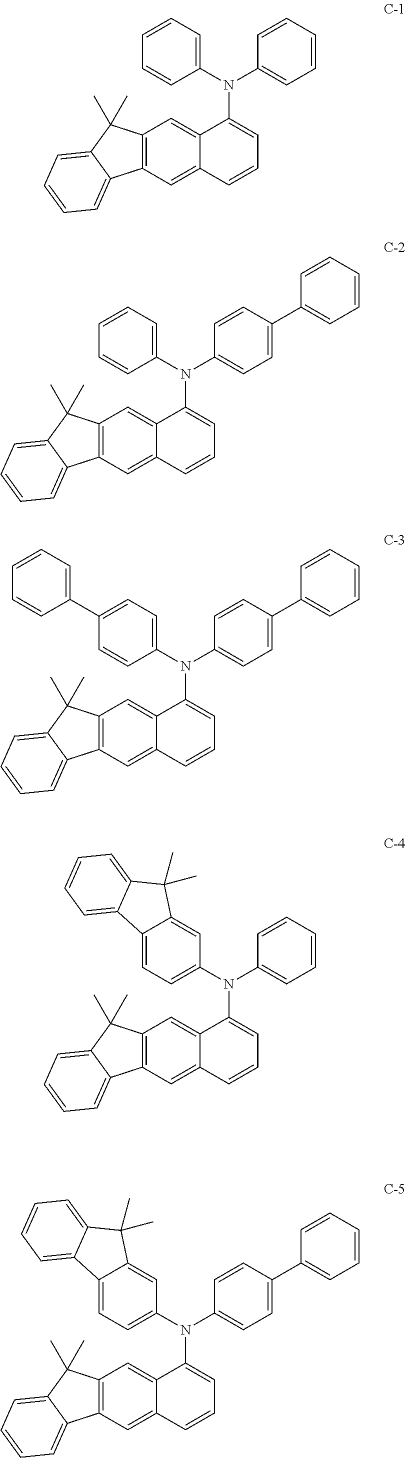 [SMILES: CC1(C)c2ccccc2-c2cc3cccc(N(c4ccccc4)c4ccccc4)c3cc21 ; CC1(C)c2ccccc2-c2cc3cccc(N(c4ccccc4)c4ccc(-c5ccccc5)cc4)c3cc21 ; CC1(C)c2ccccc2-c2cc3cccc(N(c4ccc(-c5ccccc5)cc4)c4ccc(-c5ccccc5)cc4)c3cc21 ; CC1(C)c2ccccc2-c2ccc(N(c3ccccc3)c3cccc4cc5c(cc34)C(C)(C)c3ccccc3-5)cc21 ; CC1(C)c2ccccc2-c2ccc(N(c3ccc(-c4ccccc4)cc3)c3cccc4cc5c(cc34)C(C)(C)c3ccccc3-5)cc21]

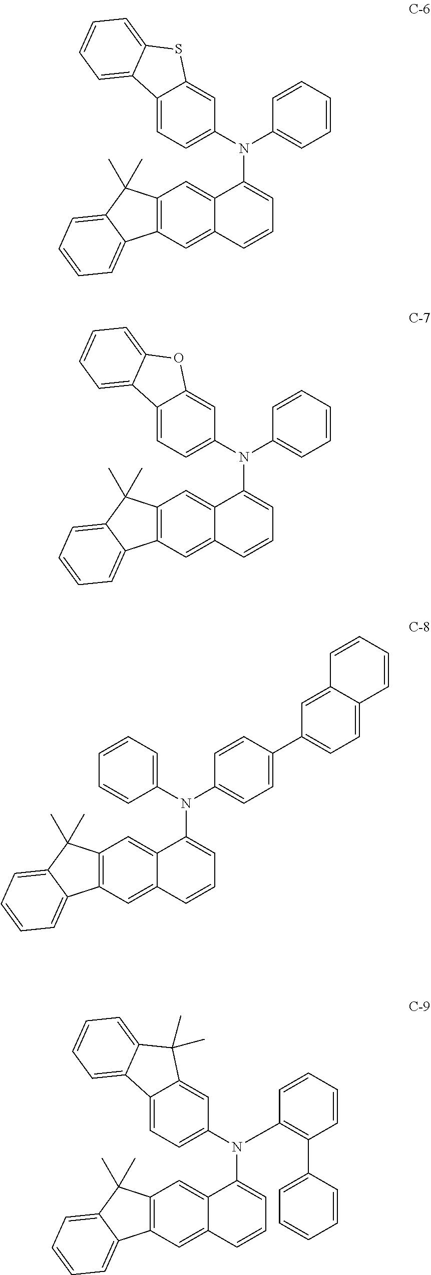 [SMILES: CC1(C)c2ccccc2-c2cc3cccc(N(c4ccccc4)c4ccc5c(c4)sc4ccccc45)c3cc21 ; CC1(C)c2ccccc2-c2cc3cccc(N(c4ccccc4)c4ccc5c(c4)oc4ccccc45)c3cc21 ; CC1(C)c2ccccc2-c2cc3cccc(N(c4ccccc4)c4ccc(-c5ccc6ccccc6c5)cc4)c3cc21 ; CC1(C)c2ccccc2-c2ccc(N(c3ccccc3-c3ccccc3)c3cccc4cc5c(cc34)C(C)(C)c3ccccc3-5)cc21]

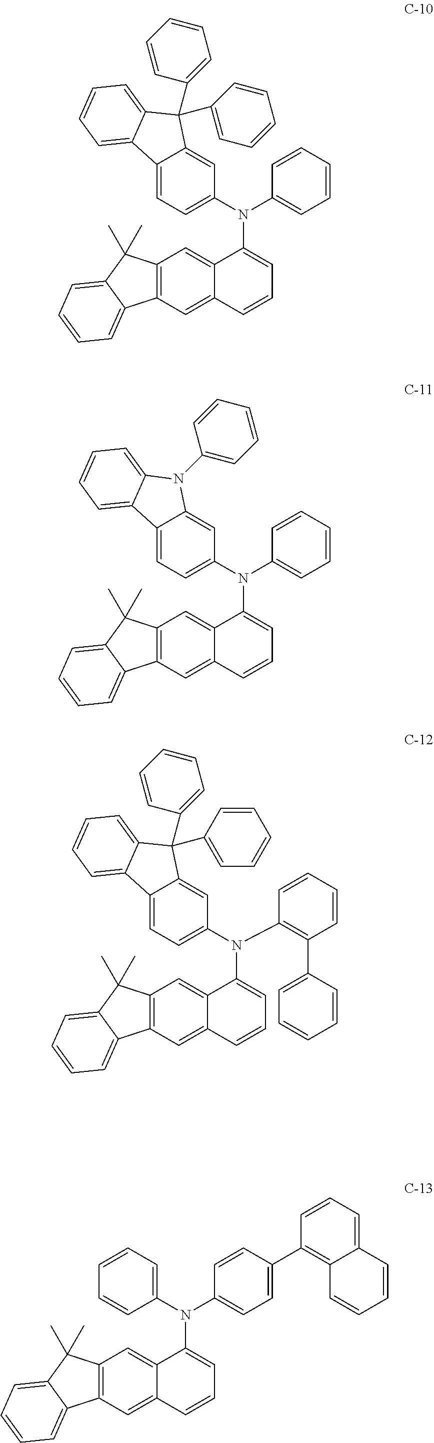 [SMILES: CC1(C)c2ccccc2-c2cc3cccc(N(c4ccccc4)c4ccc5c(c4)C(c4ccccc4)(c4ccccc4)c4ccccc4-5)c3cc21 ; CC1(C)c2ccccc2-c2cc3cccc(N(c4ccccc4)c4ccc5c6ccccc6n(-c6ccccc6)c5c4)c3cc21 ; CC1(C)c2ccccc2-c2cc3cccc(N(c4ccc5c(c4)C(c4ccccc4)(c4ccccc4)c4ccccc4-5)c4ccccc4-c4ccccc4)c3cc21 ; CC1(C)c2ccccc2-c2cc3cccc(N(c4ccccc4)c4ccc(-c5cccc6ccccc56)cc4)c3cc21]

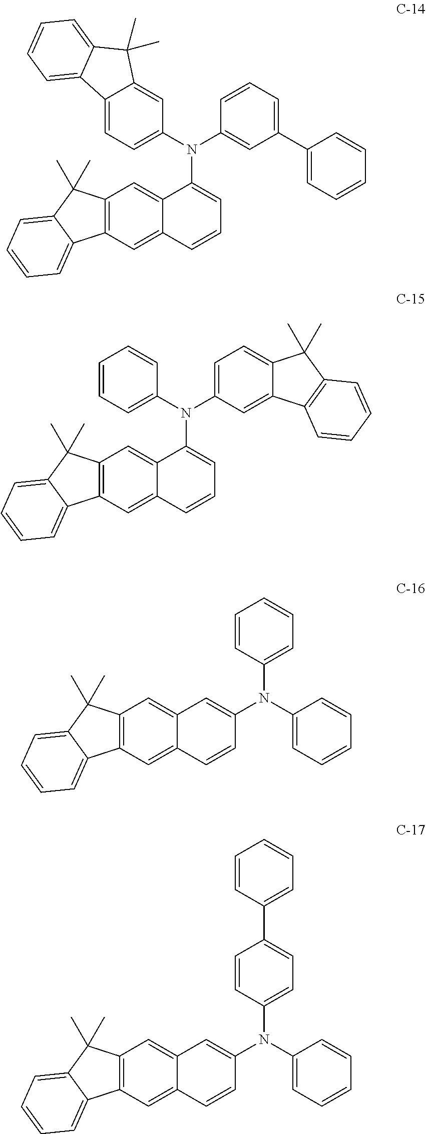 [SMILES: CC1(C)c2ccccc2-c2ccc(N(c3cccc(-c4ccccc4)c3)c3cccc4cc5c(cc34)C(C)(C)c3ccccc3-5)cc21 ; CC1(C)c2ccccc2-c2cc(N(c3ccccc3)c3cccc4cc5c(cc34)C(C)(C)c3ccccc3-5)ccc21 ; CC1(C)c2ccccc2-c2cc3ccc(N(c4ccccc4)c4ccccc4)cc3cc21 ; CC1(C)c2ccccc2-c2cc3ccc(N(c4ccccc4)c4ccc(-c5ccccc5)cc4)cc3cc21]

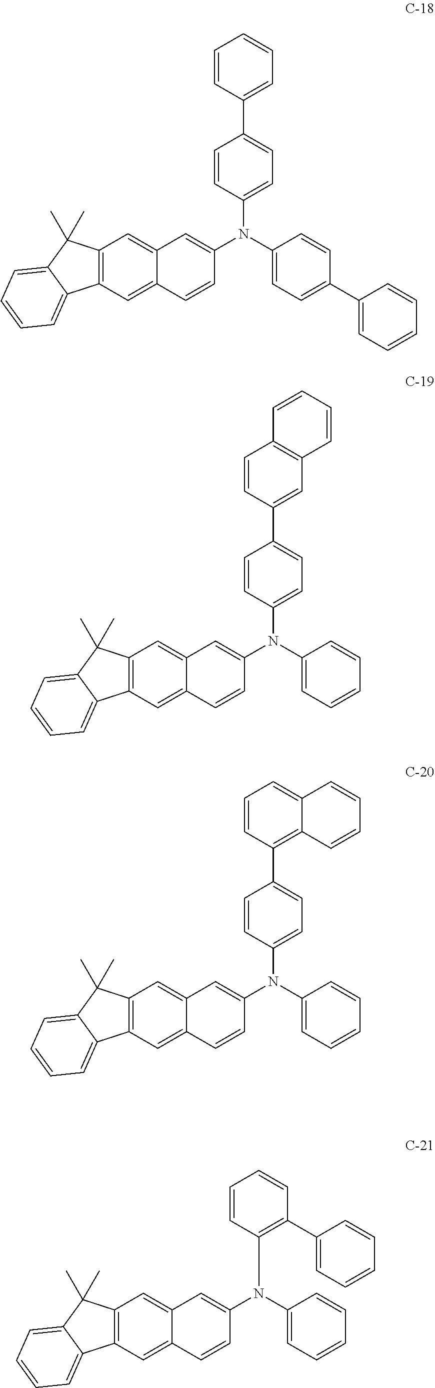 [SMILES: CC1(C)c2ccccc2-c2cc3ccc(N(c4ccc(-c5ccccc5)cc4)c4ccc(-c5ccccc5)cc4)cc3cc21 ; CC1(C)c2ccccc2-c2cc3ccc(N(c4ccccc4)c4ccc(-c5ccc6ccccc6c5)cc4)cc3cc21 ; CC1(C)c2ccccc2-c2cc3ccc(N(c4ccccc4)c4ccc(-c5cccc6ccccc56)cc4)cc3cc21 ; CC1(C)c2ccccc2-c2cc3ccc(N(c4ccccc4)c4ccccc4-c4ccccc4)cc3cc21]

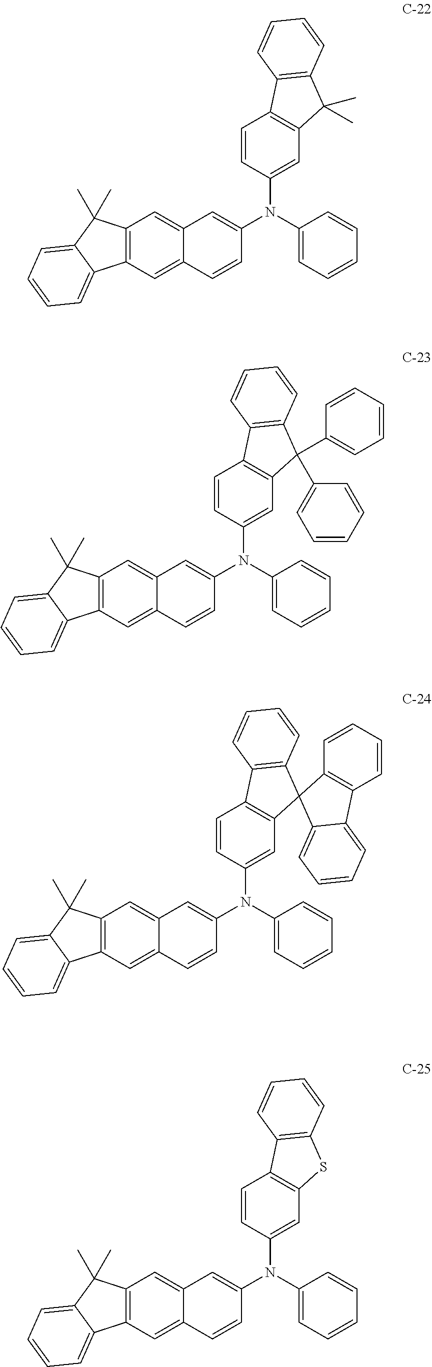 [SMILES: CC1(C)c2ccccc2-c2ccc(N(c3ccccc3)c3ccc4cc5c(cc4c3)C(C)(C)c3ccccc3-5)cc21 ; CC1(C)c2ccccc2-c2cc3ccc(N(c4ccccc4)c4ccc5c(c4)C(c4ccccc4)(c4ccccc4)c4ccccc4-5)cc3cc21 ; CC1(C)c2ccccc2-c2cc3ccc(N(c4ccccc4)c4ccc5c(c4)C4(c6ccccc6-c6ccccc64)c4ccccc4-5)cc3cc21 ; CC1(C)c2ccccc2-c2cc3ccc(N(c4ccccc4)c4ccc5c(c4)sc4ccccc45)cc3cc21]

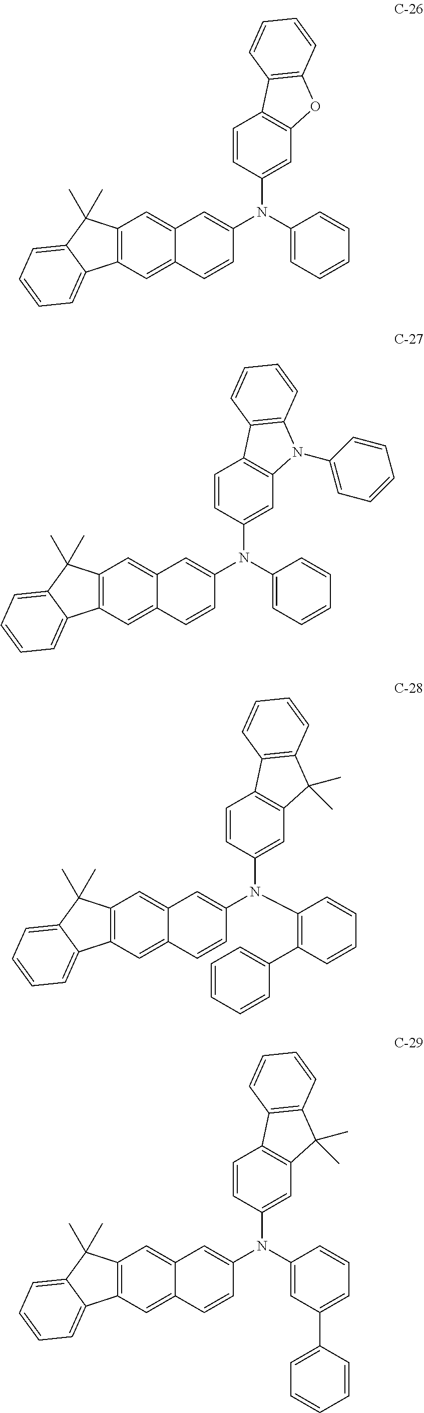 [SMILES: CC1(C)c2ccccc2-c2cc3ccc(N(c4ccccc4)c4ccc5c(c4)oc4ccccc45)cc3cc21 ; CC1(C)c2ccccc2-c2cc3ccc(N(c4ccccc4)c4ccc5c6ccccc6n(-c6ccccc6)c5c4)cc3cc21 ; CC1(C)c2ccccc2-c2ccc(N(c3ccc4cc5c(cc4c3)C(C)(C)c3ccccc3-5)c3ccccc3-c3ccccc3)cc21 ; CC1(C)c2ccccc2-c2ccc(N(c3cccc(-c4ccccc4)c3)c3ccc4cc5c(cc4c3)C(C)(C)c3ccccc3-5)cc21]

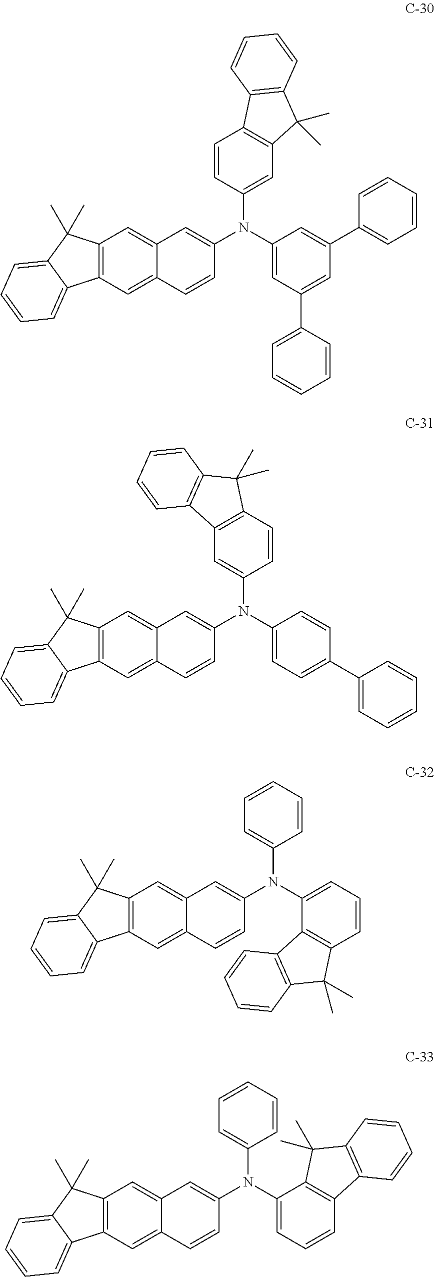 [SMILES: CC1(C)c2ccccc2-c2ccc(N(c3cc(-c4ccccc4)cc(-c4ccccc4)c3)c3ccc4cc5c(cc4c3)C(C)(C)c3ccccc3-5)cc21 ; CC1(C)c2ccccc2-c2cc(N(c3ccc(-c4ccccc4)cc3)c3ccc4cc5c(cc4c3)C(C)(C)c3ccccc3-5)ccc21 ; CC1(C)c2ccccc2-c2cc3ccc(N(c4ccccc4)c4cccc5c4-c4ccccc4C5(C)C)cc3cc21 ; CC1(C)c2ccccc2-c2cc3ccc(N(c4ccccc4)c4cccc5c4C(C)(C)c4ccccc4-5)cc3cc21]

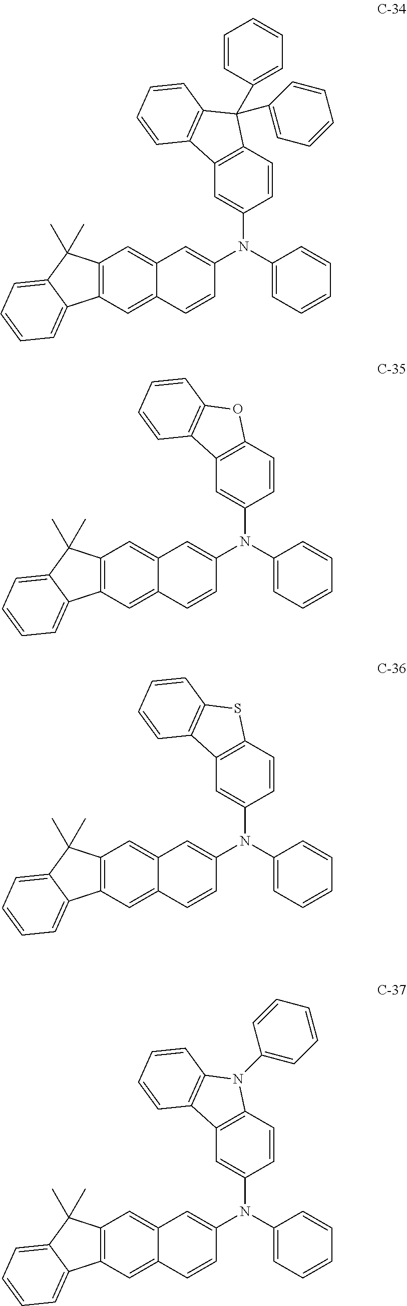 [SMILES: CC1(C)c2ccccc2-c2cc3ccc(N(c4ccccc4)c4ccc5c(c4)-c4ccccc4C5(c4ccccc4)c4ccccc4)cc3cc21 ; CC1(C)c2ccccc2-c2cc3ccc(N(c4ccccc4)c4ccc5oc6ccccc6c5c4)cc3cc21 ; CC1(C)c2ccccc2-c2cc3ccc(N(c4ccccc4)c4ccc5sc6ccccc6c5c4)cc3cc21 ; CC1(C)c2ccccc2-c2cc3ccc(N(c4ccccc4)c4ccc5c(c4)c4ccccc4n5-c4ccccc4)cc3cc21]

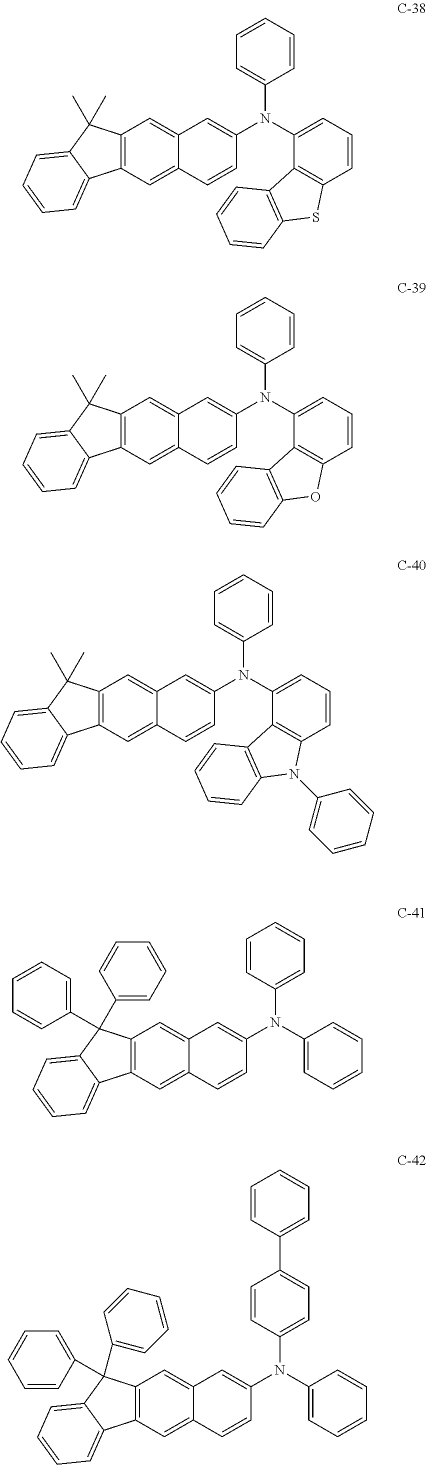 [SMILES: CC1(C)c2ccccc2-c2cc3ccc(N(c4ccccc4)c4cccc5sc6ccccc6c45)cc3cc21 ; CC1(C)c2ccccc2-c2cc3ccc(N(c4ccccc4)c4cccc5oc6ccccc6c45)cc3cc21 ; CC1(C)c2ccccc2-c2cc3ccc(N(c4ccccc4)c4cccc5c4c4ccccc4n5-c4ccccc4)cc3cc21 ; c1ccc(N(c2ccccc2)c2ccc3cc4c(cc3c2)C(c2ccccc2)(c2ccccc2)c2ccccc2-4)cc1 ; c1ccc(-c2ccc(N(c3ccccc3)c3ccc4cc5c(cc4c3)C(c3ccccc3)(c3ccccc3)c3ccccc3-5)cc2)cc1]

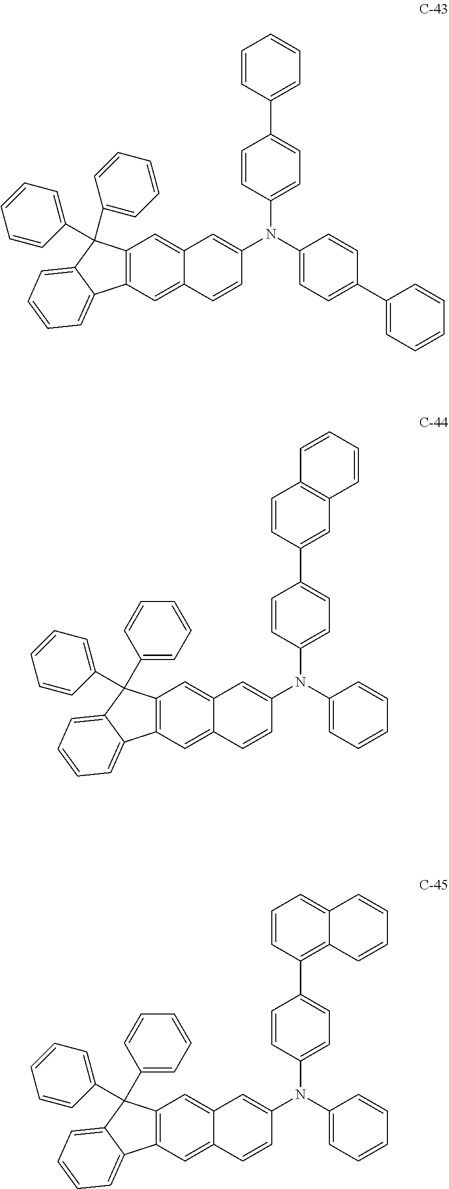 [SMILES: c1ccc(-c2ccc(N(c3ccc(-c4ccccc4)cc3)c3ccc4cc5c(cc4c3)C(c3ccccc3)(c3ccccc3)c3ccccc3-5)cc2)cc1 ; c1ccc(N(c2ccc(-c3ccc4ccccc4c3)cc2)c2ccc3cc4c(cc3c2)C(c2ccccc2)(c2ccccc2)c2ccccc2-4)cc1 ; c1ccc(N(c2ccc(-c3cccc4ccccc34)cc2)c2ccc3cc4c(cc3c2)C(c2ccccc2)(c2ccccc2)c2ccccc2-4)cc1]

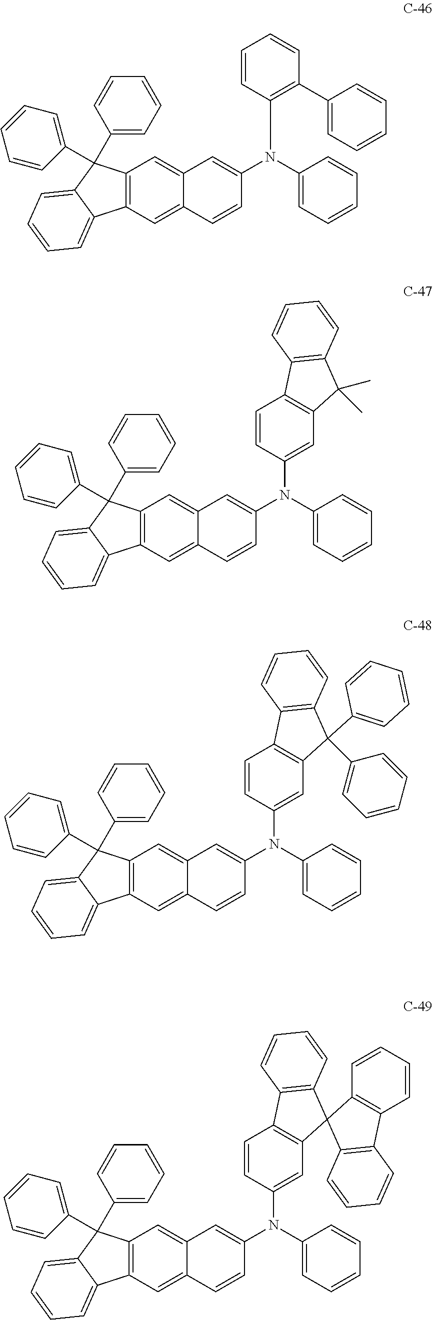 [SMILES: c1ccc(-c2ccccc2N(c2ccccc2)c2ccc3cc4c(cc3c2)C(c2ccccc2)(c2ccccc2)c2ccccc2-4)cc1 ; CC1(C)c2ccccc2-c2ccc(N(c3ccccc3)c3ccc4cc5c(cc4c3)C(c3ccccc3)(c3ccccc3)c3ccccc3-5)cc21 ; c1ccc(N(c2ccc3c(c2)C(c2ccccc2)(c2ccccc2)c2ccccc2-3)c2ccc3cc4c(cc3c2)C(c2ccccc2)(c2ccccc2)c2ccccc2-4)cc1 ; c1ccc(N(c2ccc3c(c2)C2(c4ccccc4-c4ccccc42)c2ccccc2-3)c2ccc3cc4c(cc3c2)C(c2ccccc2)(c2ccccc2)c2ccccc2-4)cc1]

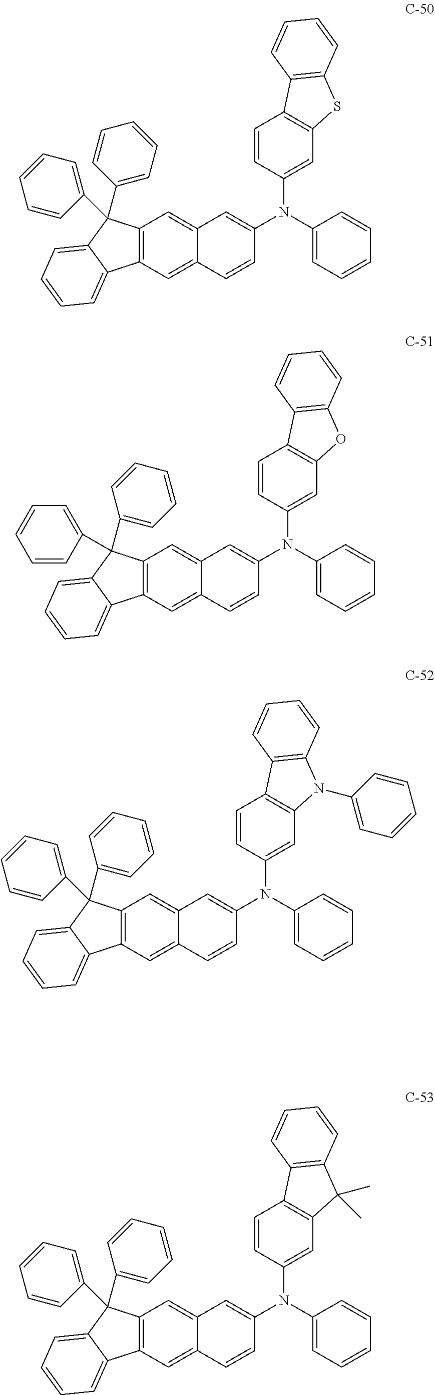 [SMILES: c1ccc(N(c2ccc3cc4c(cc3c2)C(c2ccccc2)(c2ccccc2)c2ccccc2-4)c2ccc3c(c2)sc2ccccc23)cc1 ; c1ccc(N(c2ccc3cc4c(cc3c2)C(c2ccccc2)(c2ccccc2)c2ccccc2-4)c2ccc3c(c2)oc2ccccc23)cc1 ; c1ccc(N(c2ccc3cc4c(cc3c2)C(c2ccccc2)(c2ccccc2)c2ccccc2-4)c2ccc3c4ccccc4n(-c4ccccc4)c3c2)cc1 ; CC1(C)c2ccccc2-c2ccc(N(c3ccccc3)c3ccc4cc5c(cc4c3)C(c3ccccc3)(c3ccccc3)c3ccccc3-5)cc21]

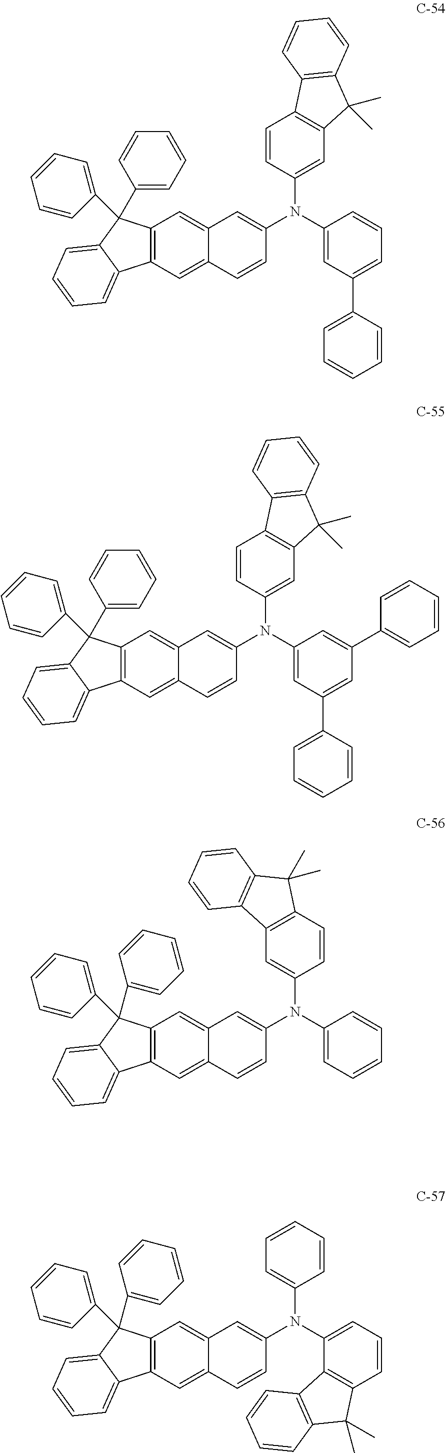 [SMILES: CC1(C)c2ccccc2-c2ccc(N(c3cccc(-c4ccccc4)c3)c3ccc4cc5c(cc4c3)C(c3ccccc3)(c3ccccc3)c3ccccc3-5)cc21 ; CC1(C)c2ccccc2-c2ccc(N(c3cc(-c4ccccc4)cc(-c4ccccc4)c3)c3ccc4cc5c(cc4c3)C(c3ccccc3)(c3ccccc3)c3ccccc3-5)cc21 ; CC1(C)c2ccccc2-c2cc(N(c3ccccc3)c3ccc4cc5c(cc4c3)C(c3ccccc3)(c3ccccc3)c3ccccc3-5)ccc21 ; CC1(C)c2ccccc2-c2c(N(c3ccccc3)c3ccc4cc5c(cc4c3)C(c3ccccc3)(c3ccccc3)c3ccccc3-5)cccc21]

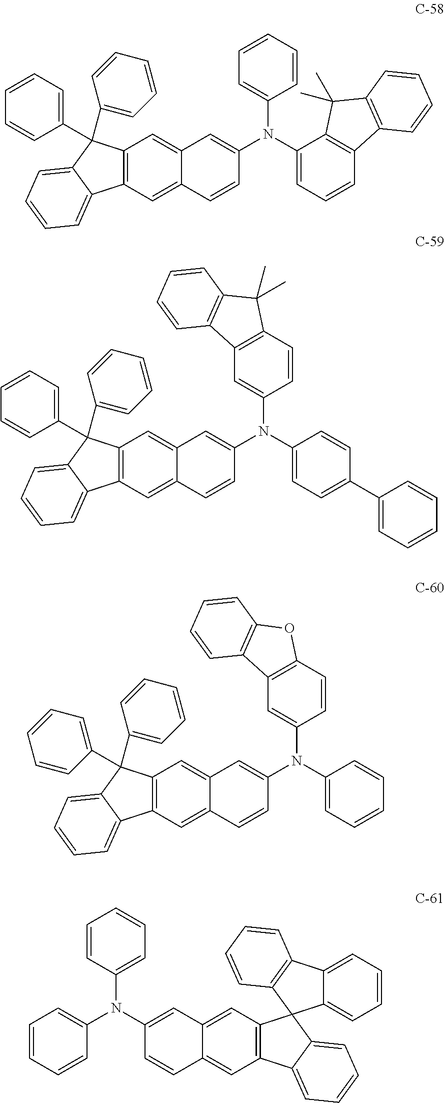 [SMILES: CC1(C)c2ccccc2-c2cccc(N(c3ccccc3)c3ccc4cc5c(cc4c3)C(c3ccccc3)(c3ccccc3)c3ccccc3-5)c21 ; CC1(C)c2ccccc2-c2cc(N(c3ccc(-c4ccccc4)cc3)c3ccc4cc5c(cc4c3)C(c3ccccc3)(c3ccccc3)c3ccccc3-5)ccc21 ; c1ccc(N(c2ccc3cc4c(cc3c2)C(c2ccccc2)(c2ccccc2)c2ccccc2-4)c2ccc3oc4ccccc4c3c2)cc1 ; c1ccc(N(c2ccccc2)c2ccc3cc4c(cc3c2)C2(c3ccccc3-c3ccccc32)c2ccccc2-4)cc1]

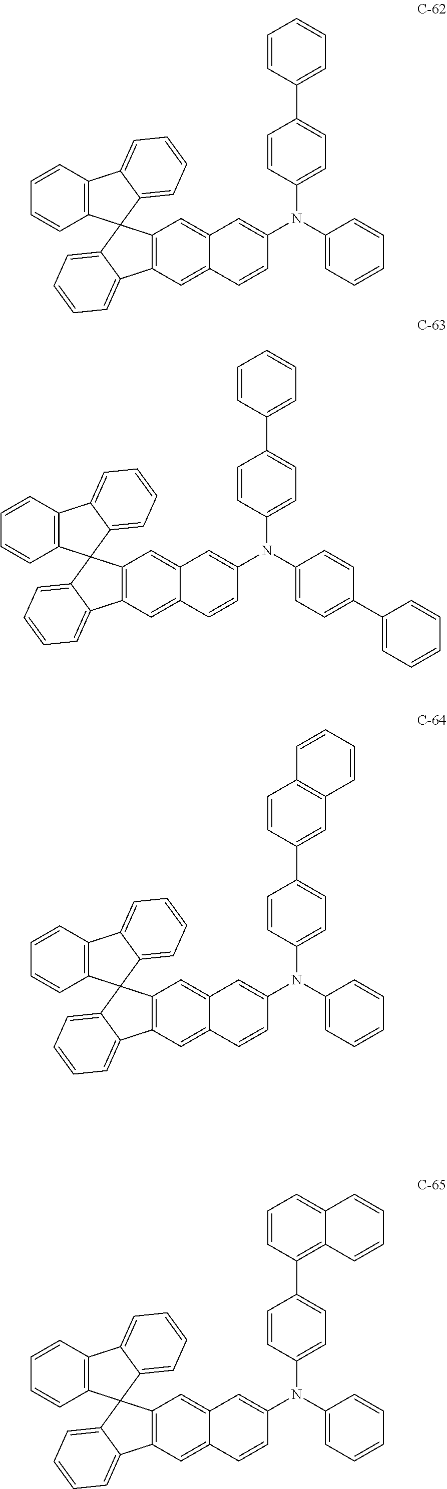 [SMILES: c1ccc(-c2ccc(N(c3ccccc3)c3ccc4cc5c(cc4c3)C3(c4ccccc4-c4ccccc43)c3ccccc3-5)cc2)cc1 ; c1ccc(-c2ccc(N(c3ccc(-c4ccccc4)cc3)c3ccc4cc5c(cc4c3)C3(c4ccccc4-c4ccccc43)c3ccccc3-5)cc2)cc1 ; c1ccc(N(c2ccc(-c3ccc4ccccc4c3)cc2)c2ccc3cc4c(cc3c2)C2(c3ccccc3-c3ccccc32)c2ccccc2-4)cc1 ; c1ccc(N(c2ccc(-c3cccc4ccccc34)cc2)c2ccc3cc4c(cc3c2)C2(c3ccccc3-c3ccccc32)c2ccccc2-4)cc1]

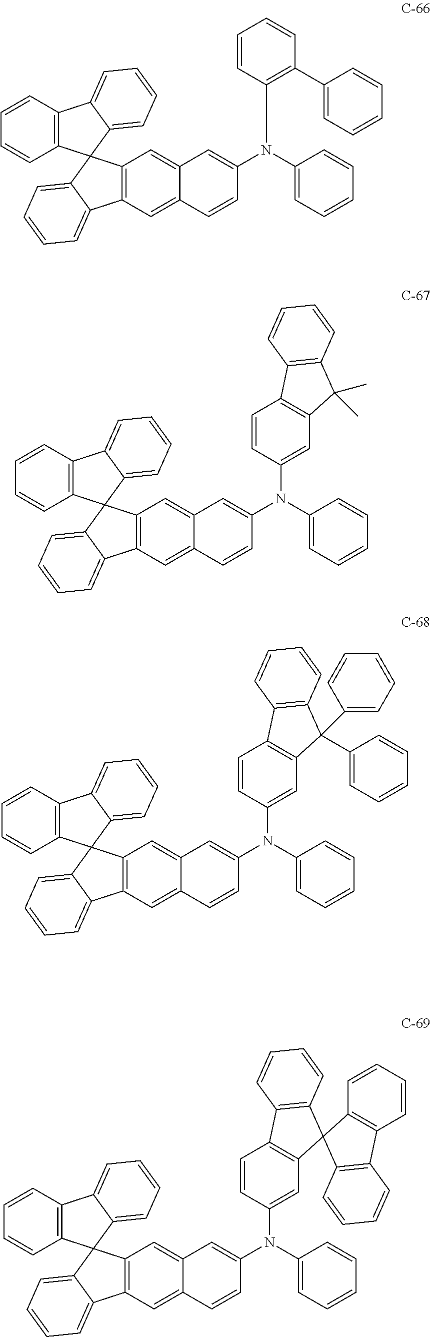 [SMILES: c1ccc(-c2ccccc2N(c2ccccc2)c2ccc3cc4c(cc3c2)C2(c3ccccc3-c3ccccc32)c2ccccc2-4)cc1 ; CC1(C)c2ccccc2-c2ccc(N(c3ccccc3)c3ccc4cc5c(cc4c3)C3(c4ccccc4-c4ccccc43)c3ccccc3-5)cc21 ; c1ccc(N(c2ccc3c(c2)C(c2ccccc2)(c2ccccc2)c2ccccc2-3)c2ccc3cc4c(cc3c2)C2(c3ccccc3-c3ccccc32)c2ccccc2-4)cc1 ; c1ccc(N(c2ccc3c(c2)C2(c4ccccc4-c4ccccc42)c2ccccc2-3)c2ccc3cc4c(cc3c2)C2(c3ccccc3-c3ccccc32)c2ccccc2-4)cc1]

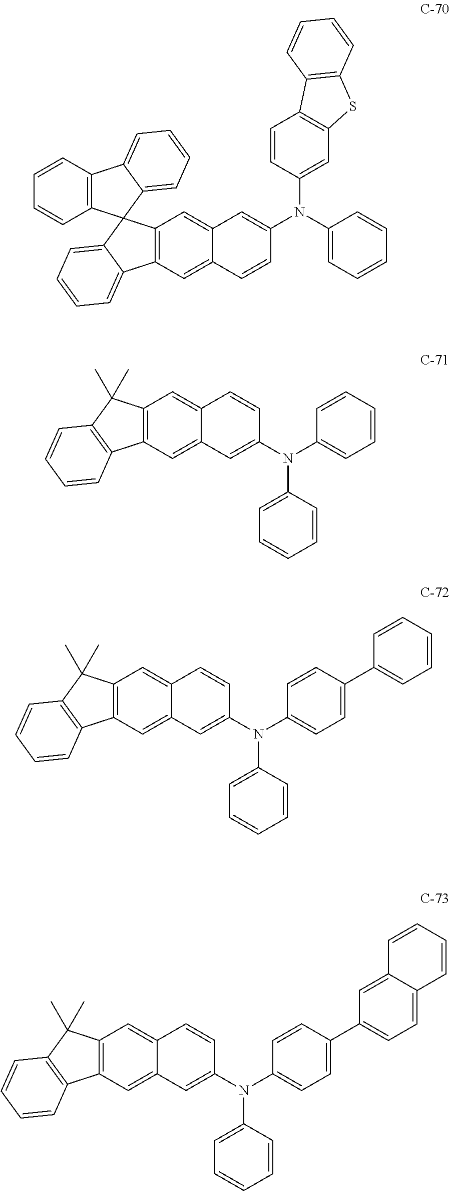 [SMILES: c1ccc(N(c2ccc3cc4c(cc3c2)C2(c3ccccc3-c3ccccc32)c2ccccc2-4)c2ccc3c(c2)sc2ccccc23)cc1 ; CC1(C)c2ccccc2-c2cc3cc(N(c4ccccc4)c4ccccc4)ccc3cc21 ; CC1(C)c2ccccc2-c2cc3cc(N(c4ccccc4)c4ccc(-c5ccccc5)cc4)ccc3cc21 ; CC1(C)c2ccccc2-c2cc3cc(N(c4ccccc4)c4ccc(-c5ccc6ccccc6c5)cc4)ccc3cc21]

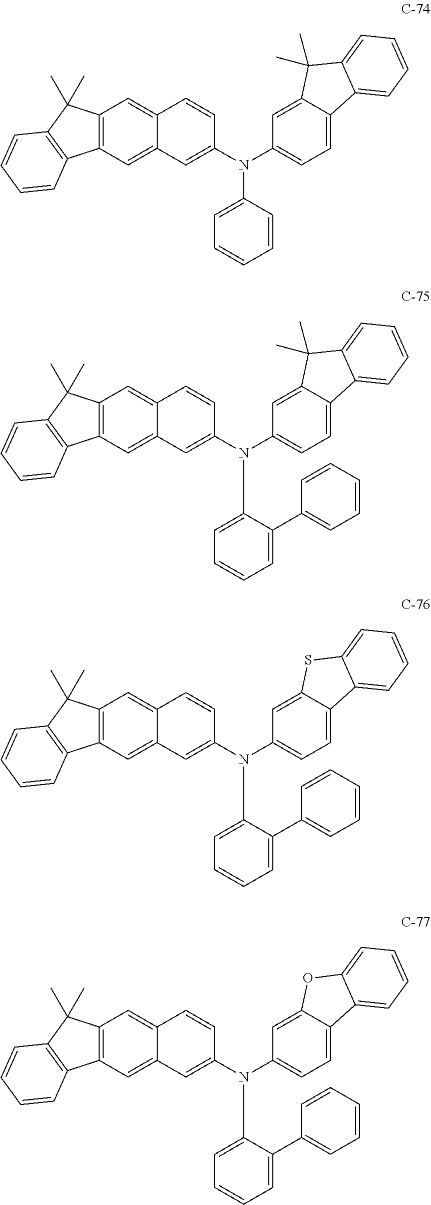 [SMILES: CC1(C)c2ccccc2-c2ccc(N(c3ccccc3)c3ccc4cc5c(cc4c3)-c3ccccc3C5(C)C)cc21 ; CC1(C)c2ccccc2-c2ccc(N(c3ccc4cc5c(cc4c3)-c3ccccc3C5(C)C)c3ccccc3-c3ccccc3)cc21 ; CC1(C)c2ccccc2-c2cc3cc(N(c4ccc5c(c4)sc4ccccc45)c4ccccc4-c4ccccc4)ccc3cc21 ; CC1(C)c2ccccc2-c2cc3cc(N(c4ccc5c(c4)oc4ccccc45)c4ccccc4-c4ccccc4)ccc3cc21]

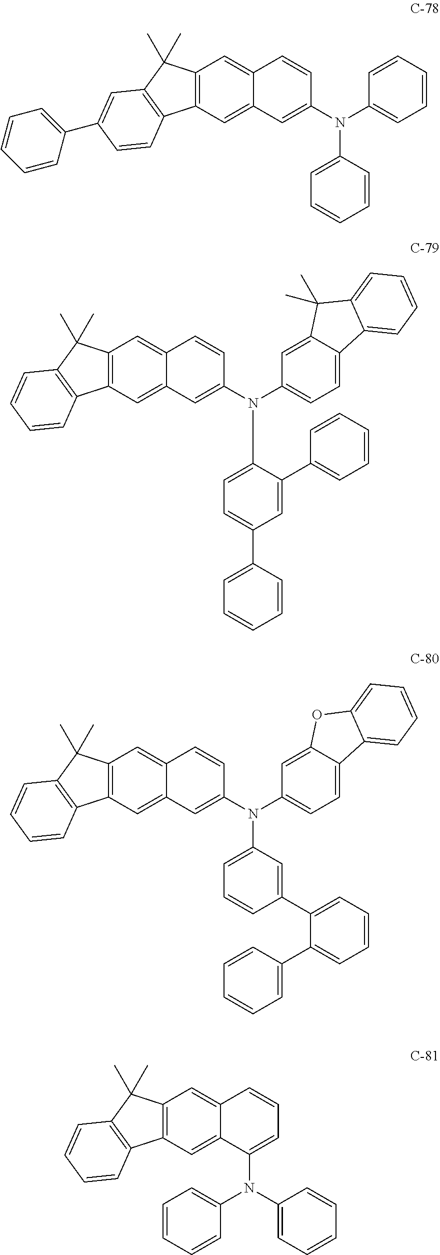 [SMILES: CC1(C)c2cc(-c3ccccc3)ccc2-c2cc3cc(N(c4ccccc4)c4ccccc4)ccc3cc21 ; CC1(C)c2ccccc2-c2ccc(N(c3ccc4cc5c(cc4c3)-c3ccccc3C5(C)C)c3ccc(-c4ccccc4)cc3-c3ccccc3)cc21 ; CC1(C)c2ccccc2-c2cc3cc(N(c4cccc(-c5ccccc5-c5ccccc5)c4)c4ccc5c(c4)oc4ccccc45)ccc3cc21 ; CC1(C)c2ccccc2-c2cc3c(N(c4ccccc4)c4ccccc4)cccc3cc21]

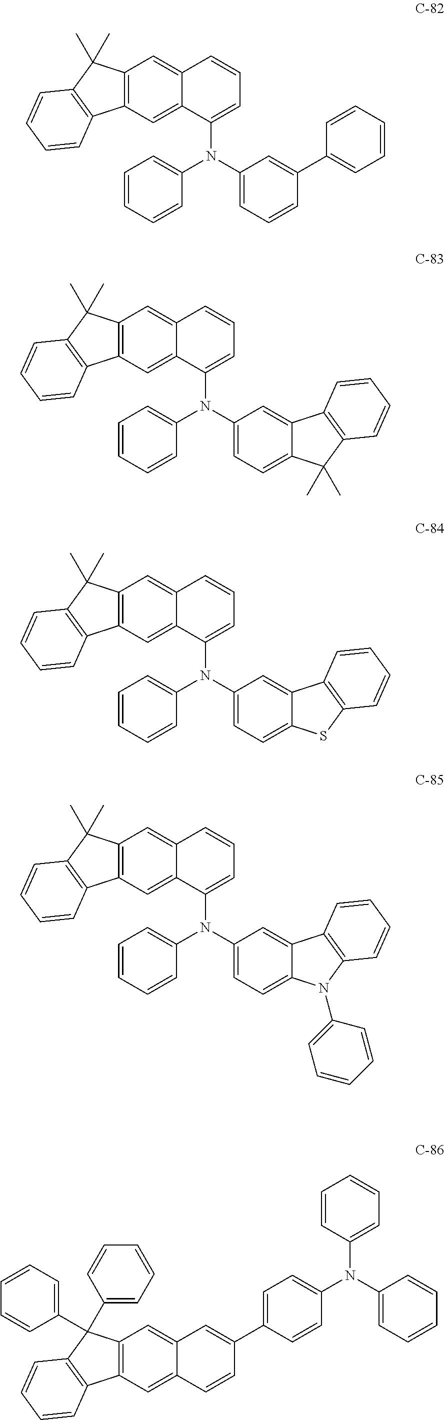 [SMILES: CC1(C)c2ccccc2-c2cc3c(N(c4ccccc4)c4cccc(-c5ccccc5)c4)cccc3cc21 ; CC1(C)c2ccccc2-c2cc(N(c3ccccc3)c3cccc4cc5c(cc34)-c3ccccc3C5(C)C)ccc21 ; CC1(C)c2ccccc2-c2cc3c(N(c4ccccc4)c4ccc5sc6ccccc6c5c4)cccc3cc21 ; CC1(C)c2ccccc2-c2cc3c(N(c4ccccc4)c4ccc5c(c4)c4ccccc4n5-c4ccccc4)cccc3cc21 ; c1ccc(N(c2ccccc2)c2ccc(-c3ccc4cc5c(cc4c3)C(c3ccccc3)(c3ccccc3)c3ccccc3-5)cc2)cc1]

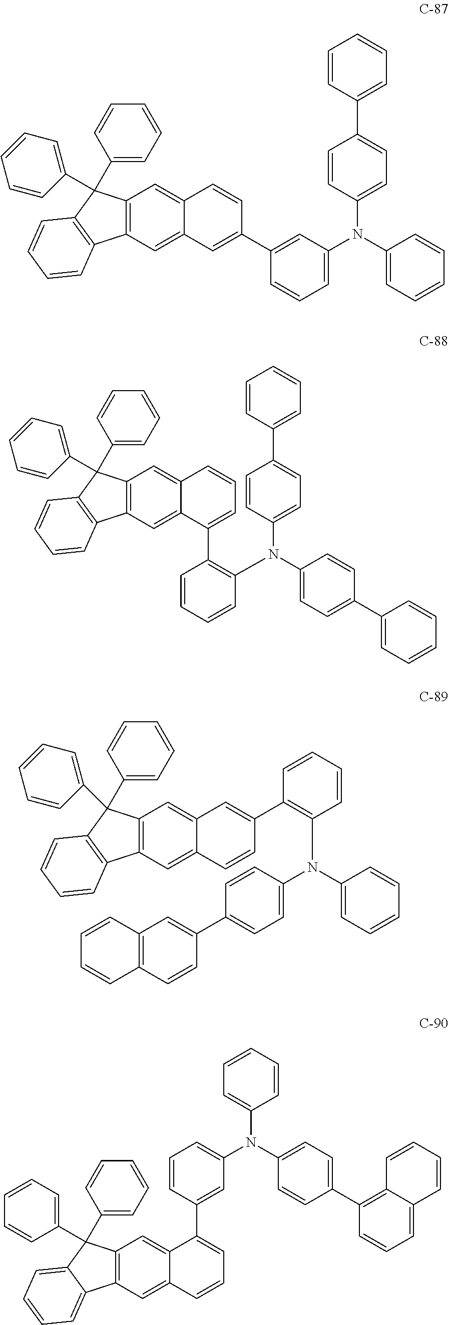 [SMILES: c1ccc(-c2ccc(N(c3ccccc3)c3cccc(-c4ccc5cc6c(cc5c4)-c4ccccc4C6(c4ccccc4)c4ccccc4)c3)cc2)cc1 ; c1ccc(-c2ccc(N(c3ccc(-c4ccccc4)cc3)c3ccccc3-c3cccc4cc5c(cc34)-c3ccccc3C5(c3ccccc3)c3ccccc3)cc2)cc1 ; c1ccc(N(c2ccc(-c3ccc4ccccc4c3)cc2)c2ccccc2-c2ccc3cc4c(cc3c2)C(c2ccccc2)(c2ccccc2)c2ccccc2-4)cc1 ; c1ccc(N(c2ccc(-c3cccc4ccccc34)cc2)c2cccc(-c3cccc4cc5c(cc34)C(c3ccccc3)(c3ccccc3)c3ccccc3-5)c2)cc1]

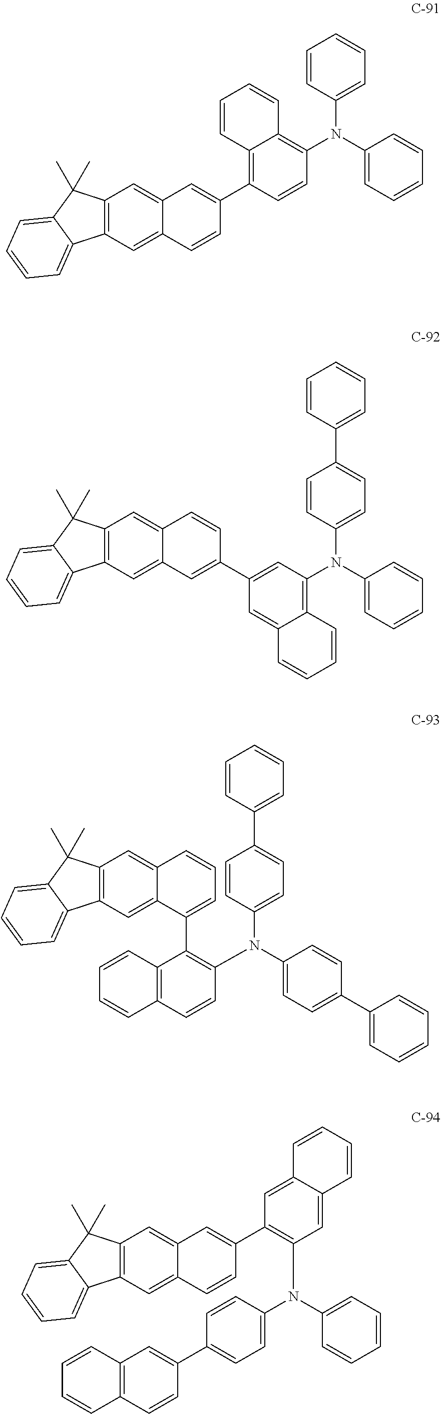 [SMILES: CC1(C)c2ccccc2-c2cc3ccc(-c4ccc(N(c5ccccc5)c5ccccc5)c5ccccc45)cc3cc21 ; CC1(C)c2ccccc2-c2cc3cc(-c4cc(N(c5ccccc5)c5ccc(-c6ccccc6)cc5)c5ccccc5c4)ccc3cc21 ; CC1(C)c2ccccc2-c2cc3c(-c4c(N(c5ccc(-c6ccccc6)cc5)c5ccc(-c6ccccc6)cc5)ccc5ccccc45)cccc3cc21 ; CC1(C)c2ccccc2-c2cc3ccc(-c4cc5ccccc5cc4N(c4ccccc4)c4ccc(-c5ccc6ccccc6c5)cc4)cc3cc21]

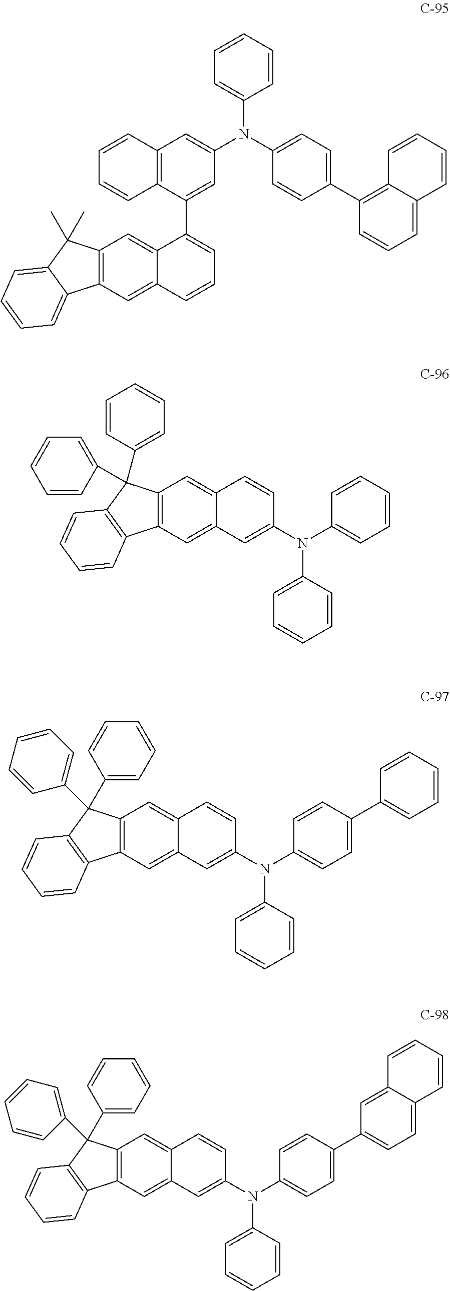 [SMILES: CC1(C)c2ccccc2-c2cc3cccc(-c4cc(N(c5ccccc5)c5ccc(-c6cccc7ccccc67)cc5)cc5ccccc45)c3cc21 ; c1ccc(N(c2ccccc2)c2ccc3cc4c(cc3c2)-c2ccccc2C4(c2ccccc2)c2ccccc2)cc1 ; c1ccc(-c2ccc(N(c3ccccc3)c3ccc4cc5c(cc4c3)-c3ccccc3C5(c3ccccc3)c3ccccc3)cc2)cc1 ; c1ccc(N(c2ccc(-c3ccc4ccccc4c3)cc2)c2ccc3cc4c(cc3c2)-c2ccccc2C4(c2ccccc2)c2ccccc2)cc1]

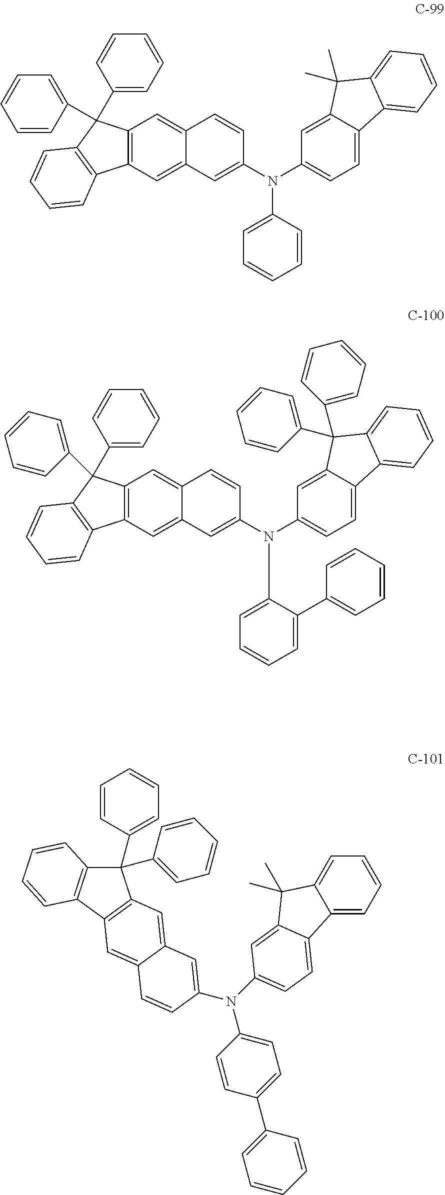 [SMILES: CC1(C)c2ccccc2-c2ccc(N(c3ccccc3)c3ccc4cc5c(cc4c3)-c3ccccc3C5(c3ccccc3)c3ccccc3)cc21 ; c1ccc(-c2ccccc2N(c2ccc3c(c2)C(c2ccccc2)(c2ccccc2)c2ccccc2-3)c2ccc3cc4c(cc3c2)-c2ccccc2C4(c2ccccc2)c2ccccc2)cc1 ; CC1(C)c2ccccc2-c2ccc(N(c3ccc(-c4ccccc4)cc3)c3ccc4cc5c(cc4c3)C(c3ccccc3)(c3ccccc3)c3ccccc3-5)cc21]

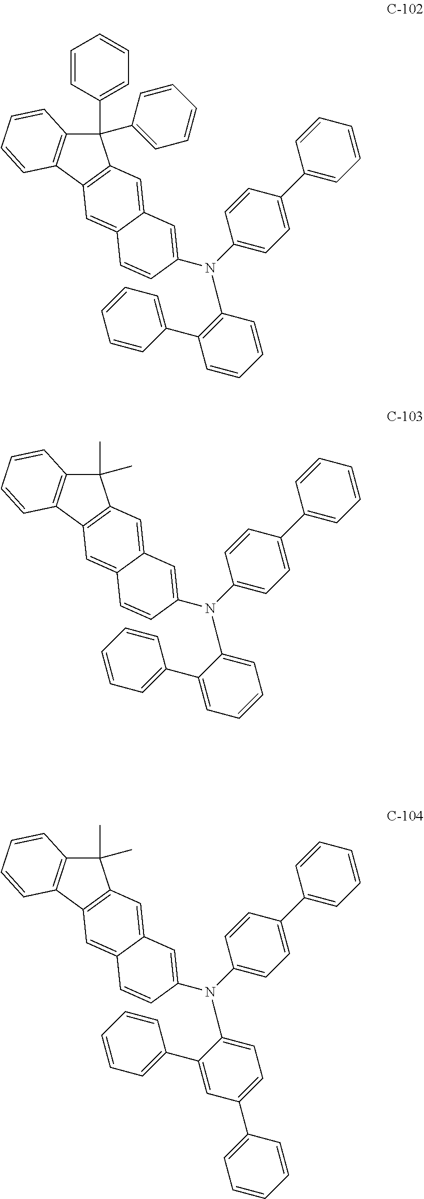 [SMILES: c1ccc(-c2ccc(N(c3ccc4cc5c(cc4c3)C(c3ccccc3)(c3ccccc3)c3ccccc3-5)c3ccccc3-c3ccccc3)cc2)cc1 ; CC1(C)c2ccccc2-c2cc3ccc(N(c4ccc(-c5ccccc5)cc4)c4ccccc4-c4ccccc4)cc3cc21 ; CC1(C)c2ccccc2-c2cc3ccc(N(c4ccc(-c5ccccc5)cc4)c4ccc(-c5ccccc5)cc4-c4ccccc4)cc3cc21]

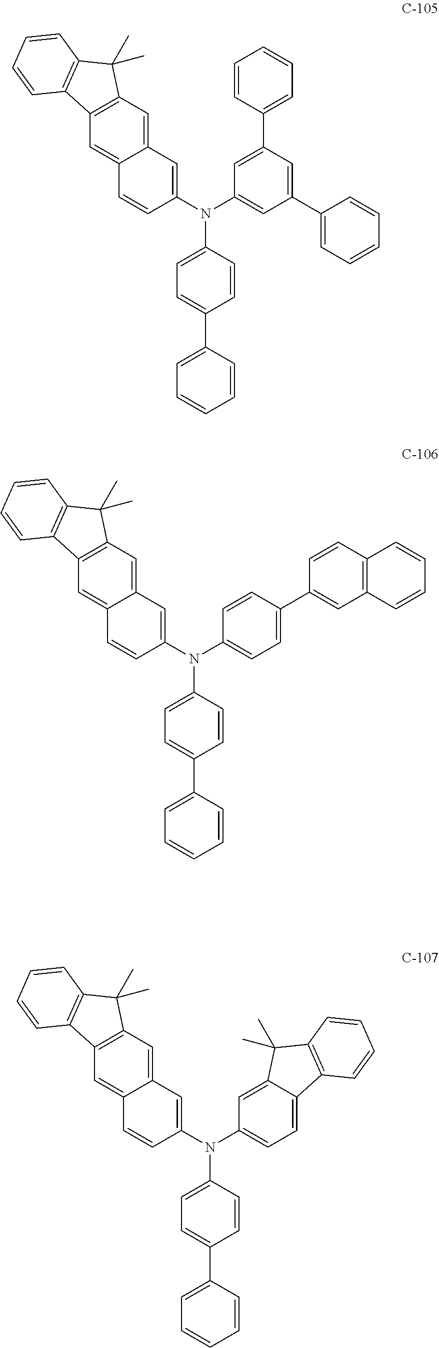 [SMILES: CC1(C)c2ccccc2-c2cc3ccc(N(c4ccc(-c5ccccc5)cc4)c4cc(-c5ccccc5)cc(-c5ccccc5)c4)cc3cc21 ; CC1(C)c2ccccc2-c2cc3ccc(N(c4ccc(-c5ccccc5)cc4)c4ccc(-c5ccc6ccccc6c5)cc4)cc3cc21 ; CC1(C)c2ccccc2-c2ccc(N(c3ccc(-c4ccccc4)cc3)c3ccc4cc5c(cc4c3)C(C)(C)c3ccccc3-5)cc21]

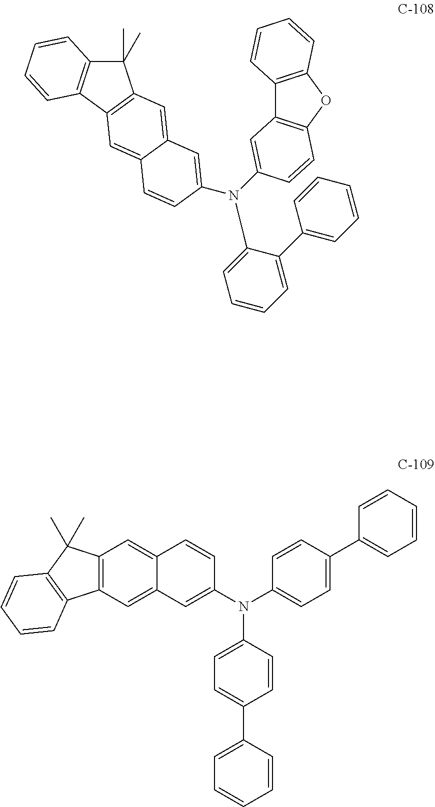 [SMILES: CC1(C)c2ccccc2-c2cc3ccc(N(c4ccc5oc6ccccc6c5c4)c4ccccc4-c4ccccc4)cc3cc21 ; CC1(C)c2ccccc2-c2cc3cc(N(c4ccc(-c5ccccc5)cc4)c4ccc(-c5ccccc5)cc4)ccc3cc21]

The organic electroluminescent compound according to the present disclosure may be prepared by a synthetic method known to one skilled in the art. For example, the organic electroluminescent compound of the present disclosure may be synthesized as shown in the following reaction schemes 1 and 2, but is not limited thereto. The organic electroluminescent compound of the present disclosure can be synthesized in large quantities by using the following synthesis method, since the starting materials are readily available and the generation of isomers can be minimized.

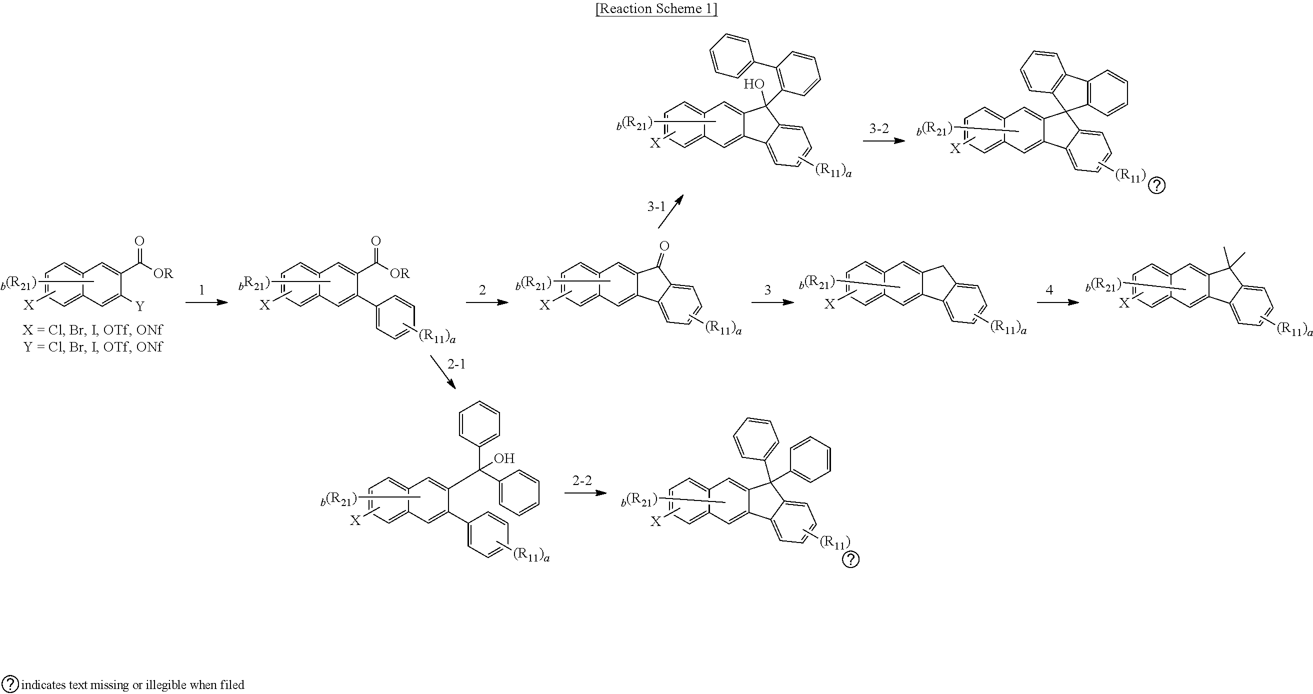

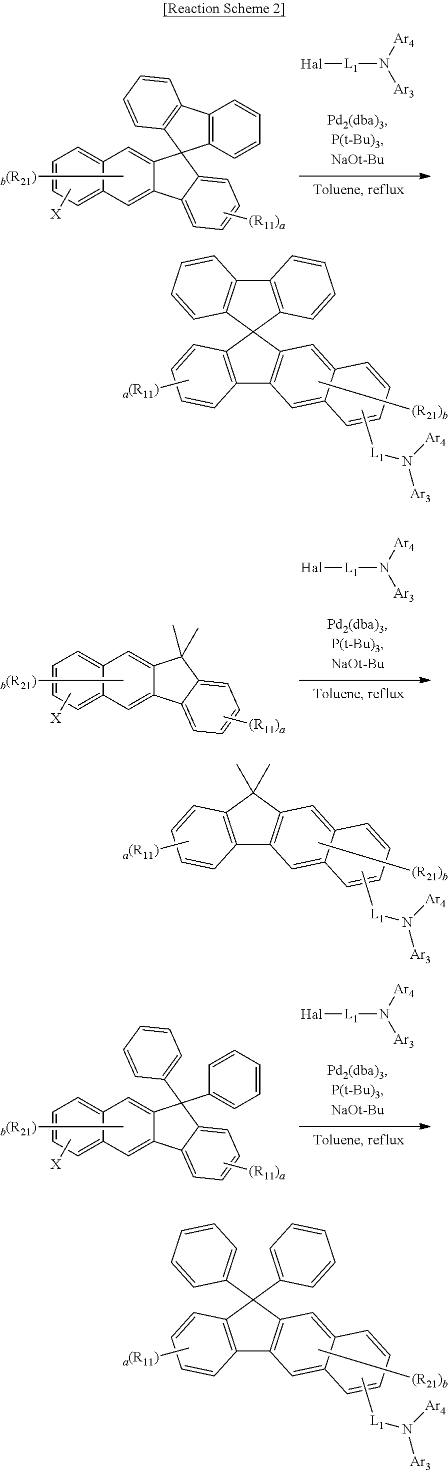

In reaction scheme 1, “1” represents a Suzuki reaction, “2” represents an intramolecular cyclization, “3” represents a reduction of ketone, “4” represents a methylation, “2-1” represents Grinard reaction, “2-2” represents an intramolecular cyclization, “3-1” represents a lithiation, and “3-2” represents an intramolecular cyclization, respectively. In reaction schemes 1 and 2, L₁, Ar₃, and Ar₄ are as defined in formula 1, a is an integer of 1 to 4, b is an integer of 1 to 5, and when a and b are each an integer of 2 or more, each of R₁₁ and each of R₂₁ may be the same as or different from each other, and the definitions of R₁ and R₂₁ are each independently the same as the definition of R₁ to R₆ in formula 1.

Although illustrative synthesis examples of the compound represented by formula 1 are described above, one skilled in the art will be able to readily understand that all of them are based on a Buchwald-Hartwig cross-coupling reaction, an N-arylation reaction, an H-mont-mediated etherification reaction, a Miyaura borylation reaction, a Suzuki cross-coupling reaction, an Intramolecular acid-induced cyclization reaction, a Pd(II)-catalyzed oxidative cyclization reaction, a Grignard reaction, a Heck reaction, a Cyclic Dehydration reaction, an SN₁ substitution reaction, an SN₂ substitution reaction, a Phosphine-mediated reductive cyclization reaction, etc., and the reactions above proceed even when substituents, which are defined in formula 1 above but are not specified in the specific synthesis examples, are bonded.

Dopants that can be used in combination with the compounds of the present disclosure may be at least one phosphorescent or fluorescent dopant, preferably a phosphorescent dopant. The phosphorescent dopant is not particularly limited, but may be a complex compound of metal atoms selected from iridium (Ir), osmium (Os), copper (Cu), and platinum (Pt), and in some cases, preferably, may be an ortho-metalized complex compound of a metal atom selected from iridium (Ir), osmium (Os), copper (Cu), and platinum (Pt), and in some cases, more preferably, may be an ortho-metalized iridium complex compound.

The organic electroluminescent compound represented by formula 1 of the present disclosure may be comprised in at least one layer constituting an organic electroluminescent device, for example, may be comprised in at least one layer selected from a hole injection layer, a hole transport layer, a hole auxiliary layer, a light-emitting auxiliary layer, a light-emitting layer, an electron transport layer, an electron buffer layer, an electron injection layer, an interlayer, a hole blocking layer, and an electron blocking layer. Each of the layers may be further constituted with multiple layers. In addition, the compound represented by formula 1 of the present disclosure may be comprised in at least one layer of a hole injection layer, a hole transport layer, a hole auxiliary layer, a light-emitting auxiliary layer, and an electron transport layer, but is not limited thereto.

The organic electroluminescent materials of the present disclosure, for example, at least one material of a hole injection material, a hole transport material, a hole auxiliary material, a light-emitting auxiliary material, an electron blocking material, a light-emitting material, an electron buffer material, a hole blocking material, an electron transport material, and an electron injection material may comprise a compound represented by formula 1. The material may be a material of a hole transport zone, and the material of the hole transport zone may be at least one of a hole injection material, a hole transport material, a hole auxiliary material, a light-emitting auxiliary material, and an electron blocking material. The material of the hole transport zone may consist of the compound represented by formula 1 alone, or may further comprise conventional materials comprised in the organic electroluminescent material. When two or more types of materials are comprised in one layer, the layer may be formed by mixed deposition or by co-deposition separately at the same time. When the high refractive index material of the present disclosure is used, a thickness of the device can be reduced, and thus can promote to reduce materials and improve process productivity. In particular, since the red light-emitting organic electroluminescent device is generally thicker than the blue or green light-emitting device, the use of the high refractive index material of the present disclosure for the red light-emitting device can reduce its thickness, thereby greatly improving the efficiency for manufacturing the device.

The organic electroluminescent device according to the present disclosure comprises a first electrode, a second electrode, and at least one organic layer between the first and second electrodes. One of the first and second electrodes may be an anode, and the other may be a cathode. The organic layer may comprise at least one light-emitting layers, and may further comprise at least one layer selected from a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer.

The present disclosure may comprise a hole transport zone between the anode and the light-emitting layer, and the hole transport zone may comprise at least one of a hole injection layer, a hole transport layer, a hole auxiliary layer, a light-emitting auxiliary layer, and an electron blocking layer. The hole injection layer, hole transport layer, hole auxiliary layer, light-emitting auxiliary layer, and electron blocking layer each may be a single layer or multiple layers in which two or more layers are stacked. For a hole injection layer, a plurality of layers may be used for the purpose of lowering a hole injection barrier (or a hole injection voltage) from the anode to the hole transport layer or the electron blocking layer, wherein two compounds may be used simultaneously for each layer. An electron blocking layer may be located between the hole transport layer (or the hole injection layer) and the light-emitting layer, and may block the overflow of electrons from the light-emitting layer to confine excitons in the light-emitting layer, thereby preventing a leakage of light.

In addition, the hole transport zone may comprise a p-doped hole injection layer, a hole transport layer, and a light-emitting auxiliary layer. Herein, the p-doped hole injection layer means a hole injection layer doped with a p-dopant. The p-dopant is a material which leads to have p-type semiconductor properties. The p-type semiconductor properties refer to the properties of receiving or transferring holes at the highest occupied molecule orbital (HOMO) energy level, that is, a property of a material having a high hole conductivity.

The organic electroluminescent device according to the present disclosure may comprise the compound represented by formula 1, or may further comprise conventional materials comprised in the organic electroluminescent device. The organic electroluminescent device according to the present disclosure may comprise the compound represented by formula 1 in the layer of a hole transport zone. For example, the organic electroluminescent device according to the present disclosure may comprise the compound of the present disclosure in at least one of a hole injection layer, a hole transport layer, a hole auxiliary layer, a light-emitting auxiliary layer, and an electron blocking layer.

Organic electroluminescent devices are classified into bottom emission device and top emission device according to their light-emitting structure. Specifically, the bottom emission device is the structure in which most of the light is directed toward the thin film transistor (TFT) in the organic electroluminescent device, and the top emission device is the structure in which most of the light is output to the opposite side of the thin film transistor in the organic electroluminescent device. Preferably, the organic electroluminescent device of the present disclosure may be applied to a top emission device. However, the organic electroluminescent device of the present disclosure is not limited thereto, and may be also applied to a bottom emission device.

The present disclosure can achieve the desired CIE 1931 color coordinate while optimizing a resonance phenomenon with a thin thickness by using a material exhibiting a high refractive index, thereby providing an organic electroluminescent device having an advantage in a process.

In addition, the present disclosure may provide a display system by using the compound represented by formula 1. That is, it is possible to produce a display system or a lighting system by using the compound of the present disclosure. Specifically, it is possible to produce a display system, e.g., a display system for smartphones, tablets, notebooks, PCs, TVs, or cars, or a lighting system, e.g., an outdoor or indoor lighting system, by using the compound of the present disclosure.

Hereinafter, the preparation method of the compound of the present disclosure and the properties thereof will be explained with reference to the representative compounds of the present disclosure in order to understand the present disclosure in detail. However, the present disclosure is not limited to the following examples.

### Example 1: Preparation of Compound C-28

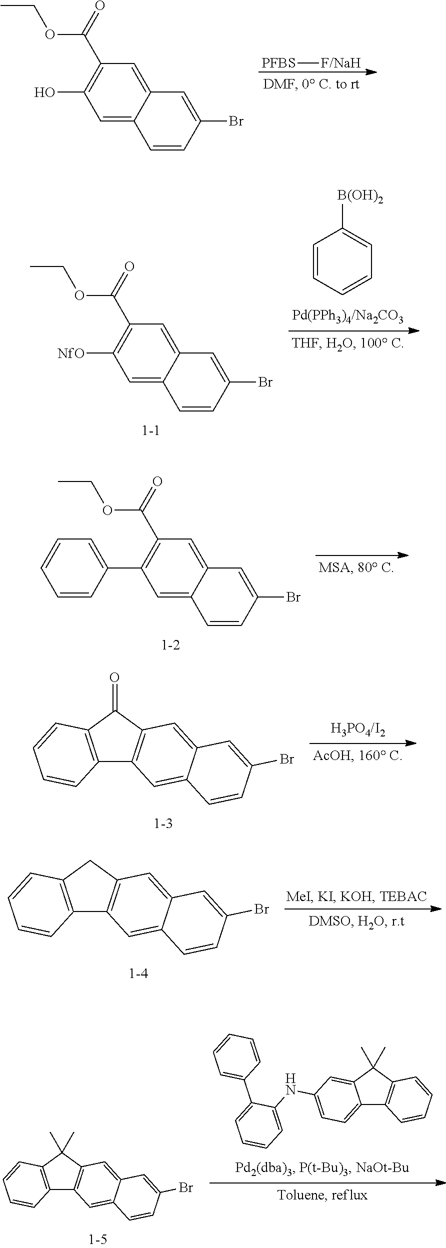

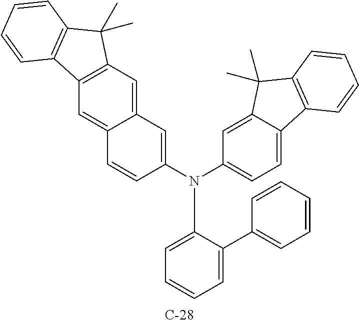

1) Synthesis of Compound 1-1

Ethyl 7-bromo-3-hydroxy-2-naphthoate (100 g, 338.8 mmol) and 1,700 mL of N,N-dimethylformamide were added to a reaction vessel, and sodium hydride (20.3 g) was slowly added at 0° C. After stirring for 30 minutes, perfluorobutanesulfonylfluoride (72 mL, 406.6 mmol) was added and stirred at room temperature for 4 hours. After the reaction was completed, methanol was added to the mixture, and the mixture was added to 2,000 mL of water to form a solid. The resulting solid was filtered under reduced pressure and washed with methanol to obtain compound 1-1 (157 g, yield: 92%).

2) Synthesis of Compound 1-2

Compound 1-1 (157.0 g, 271.9 mmol), phenylboronic acid (33.1 g, 271.9 mmol), tetrakis(triphenylphosphine)palladium(0) (9.45 g, 8.157 mmol), sodium carbonate (72 g, 679.75 mmol), 1,350 mL of tetrahydrofuran, and 340 mL of water were added to a reaction vessel, followed by stirring at 100° C. for 4 hours. After the reaction was completed, the mixture was washed with distilled water and the organic layer was extracted with ethyl acetate. The organic layer was dried with magnesium sulfate, the solvent was removed by a rotary evaporator, and then purified by column chromatography to obtain compound 1-2 (52 g, yield: 53%).

3) Synthesis of Compound 1-3

Compound 1-2 (52 g, 146.38 mmol) and 585 mL of methanesulfonic acid were added to a reaction vessel, followed by stirring at 60° C. for 5 hours. After the reaction was completed, the stirred mixture was added to 1,000 mL of water to form a solid. The resulting solid was filtered under reduced pressure and washed with methanol to obtain compound 1-3 (41.4 g, yield: 92%).

4) Synthesis of Compound 1-4

Phosphoric acid (36 mL, 214.6 mmol), iodine (17.7 g, 69.63 mmol), and acetic acid (330 mL) were added to a reaction vessel, followed by stirring at 160° C. for 10 minutes. Compound 1-3 (41.4 g, 133.91 mmol) was dissolved in 340 mL of acetic acid, and then added to the stirred mixture and stirred for 2 hours. After the reaction was completed, the mixture was added to 1,000 mL of water to form a solid. The resulting solid was filtered under reduced pressure and washed with methanol to obtain compound 1-4 (32.5 g, yield: 82%).

5) Synthesis of Compound 1-5

Compound 1-4 (32.5 g, 110.1 mmol), potassium iodide (1.83 g, 11.01 mmol), potassium hydroxide (30.89 g, 550.5 mmol), triethylbenzylammonium chloride (1.25 g, 5.505 mmol), 550 mL of dimethyl sulfoxide, and 55 mL of water were added to a reaction vessel and stirred for 1 hour. Methyl iodide was added and stirred at room temperature for 24 hours. After the reaction was completed, the mixture was washed with distilled water, and the organic layer was extracted with dichloromethane. The organic layer was dried with magnesium sulfate, the solvent was removed by a rotary evaporator, and then purified by column chromatography to obtain compound 1-5 (20 g, yield: 56%).

6) Synthesis of Compound C-28

Compound 1-5 (3.0 g, 9.28 mmol), N-([1,1′-biphenyl]-2-yl)-9,9′-dimethyl-9H-fluoren-2-amine (2.8 g, 7.73 mmol), tris(dibenzylideneacetone)dipalladium(0) (0.35 g, 0.39 mmol), tri-tert-butylphosphine (0.4 mL, 0.77 mmol), sodium ter-butoxide (1.1 g, 11.6 mmol), and 40 mL of toluene were added to a reaction vessel and stirred under reflux for 1 hour. After cooling the reaction mixture to room temperature, the solid was filtered and washed with ethyl acetate. The filtrate was distilled under reduced pressure and purified by column chromatography to obtain compound C-28 (1.9 g, yield: 34%).

| | MW | M.P. |
|---|---|---|
| C-28 | 603.30 | 238° C. |

### Example 2: Preparation of Compound C-22

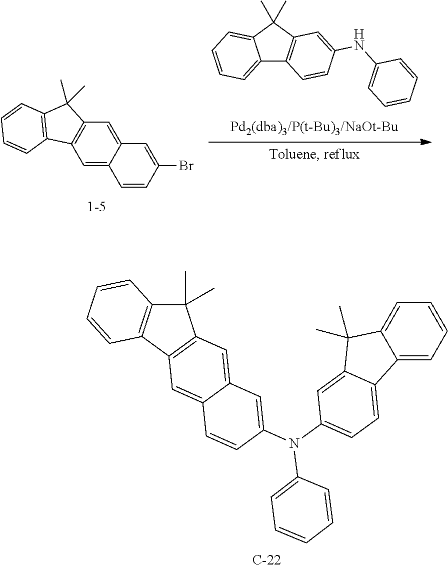

Compound 1-5 (3.0 g, 9.28 mmol), 9,9′-dimethyl-N-phenyl-9H-fluoren-2-amine (2.9 g, 10.21 mmol), tris(dibenzylideneacetone)dipalladium(0) (0.35 g, 0.39 mmol), tri-tert-butylphosphine (0.4 mL, 0.77 mmol), sodium tert-butoxide (1.1 g, 11.6 mmol), and 40 mL of toluene were added to a reaction vessel and stirred under reflux for 1 hour. After cooling the reaction mixture to room temperature, the solid was filtered and washed with ethyl acetate. The filtrate was distilled under reduced pressure and purified by column chromatography to obtain compound C-22 (2.4 g, yield: 49%).

| | MW | M.P. |
|---|---|---|
| C-22 | 527.30 | 246° C. |

### Example 3: Preparation of Compound C-18

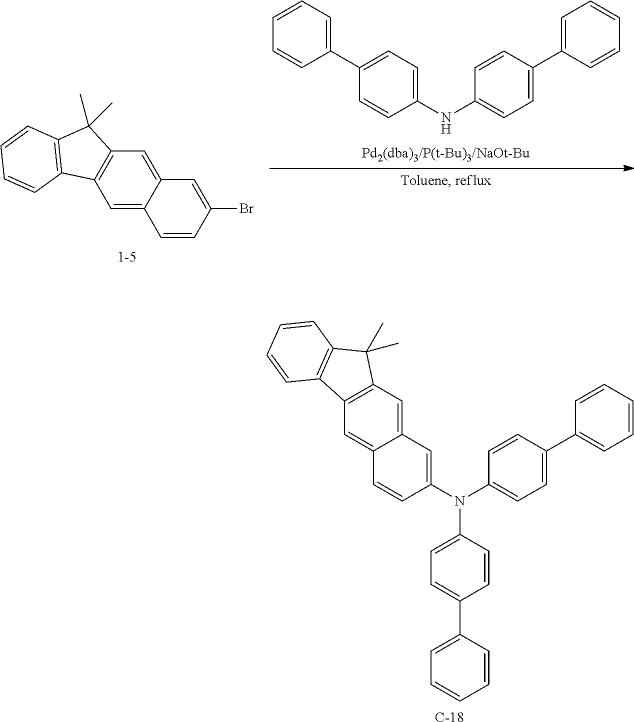

Compound 1-5 (3.0 g, 9.28 mmol), di([1,1′-biphenyl]-4-yl)amine (3.3 g, 10.21 mmol), tris(dibenzylideneacetone)dipalladium(0) (0.35 g, 0.39 mmol), tri-tert-butylphosphine (0.4 mL, 0.77 mmol), sodium tert-butoxide (1.1 g, 11.6 mmol), and 40 mL of toluene were added to a reaction vessel and stirred under reflux for 1 hour. After cooling the reaction mixture to room temperature, the solid was filtered and washed with ethyl acetate. The filtrate was distilled under reduced pressure and purified by column chromatography to obtain compound C-18 (1.4 g, yield: 26%).

| | MW | M.P. |
|---|---|---|
| C-18 | 563.30 | 239° C. |

### Example 4: Preparation of Compound C-19

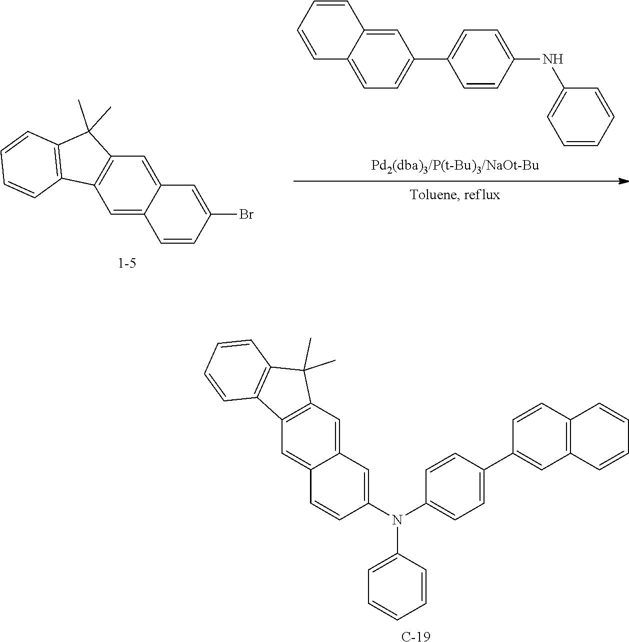

Compounds 1-5 (3.0 g, 9.28 mmol), 4-(naphthalen-2-yl)-N-phenylaniline (2.7 g, 9.28 mmol), tris(dibenzylideneacetone)dipalladium(0) (0.4 g, 0.46 mmol), tri-tert-butylphosphine (0.5 mL, 0.93 mmol), sodium tert-butoxide (1.3 g, 13.92 mmol), and 46 mL of toluene were added to a reaction vessel and stirred under reflux for 1 hour. After cooling the reaction mixture to room temperature, the solid was filtered and washed with ethyl acetate. The filtrate was distilled under reduced pressure and purified by column chromatography to obtain compound C-19 (3.1 g, yield: 62%).

| | MW | M.P. |
|---|---|---|
| C-19 | 537.71 | 170° C. |

### Example 5: Preparation of Compound C-31

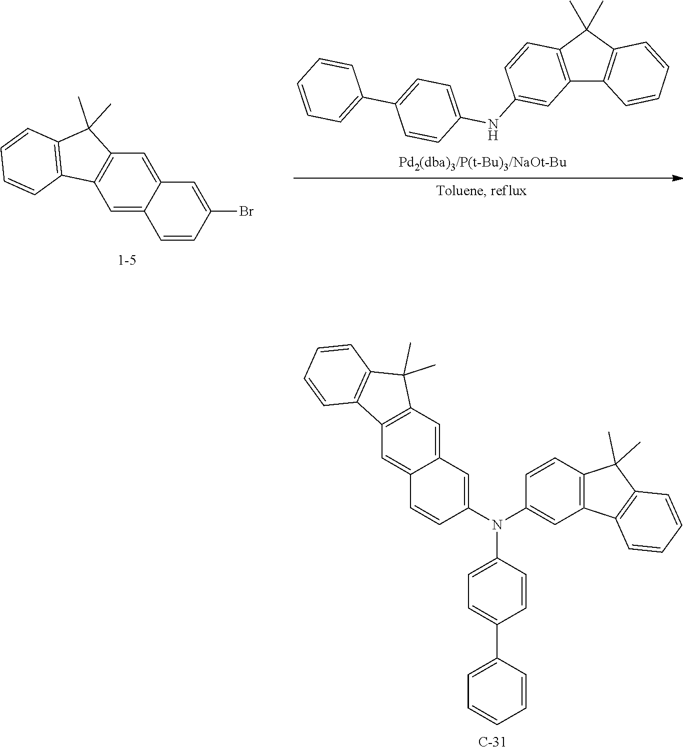

Compound 1-5 (3.0 g, 9.28 mmol), N-([1,1′-biphenyl]-4-yl)-9,9′-dimethyl]-9H-fluoren-3-amine (3.4 g, 9.28 mmol), tris(dibenzylideneacetone)dipalladium(0) (0.4 g, 0.46 mmol), tri-tert-butylphosphine (0.5 mL, 0.93 mmol), sodium tert-butoxide (1.3 g, 13.92 mmol), and 46 mL of toluene were added to a reaction vessel and stirred under reflux for 1 hour. After cooling the reaction mixture to room temperature, the solid was filtered and washed with ethyl acetate. The filtrate was distilled under reduced pressure and purified by column chromography to obtain compound C-31 (4.1 g, yield: 73%).

| | MW | M.P. |
|---|---|---|
| C-31 | 603.81 | 223° C. |

### Example 6: Preparation of Compound C-103

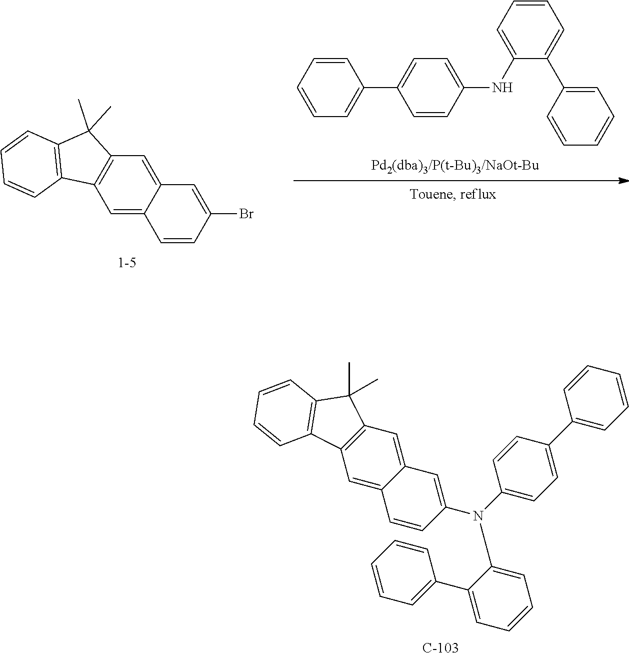

Compound 1-5 (2.0 g, 6.19 mmol), N-([1,1′-biphenyl]-4-yl)-{1,1′-biphenyl}-2-amine (1.8 g, 5.63 mmol), tris(dibenzylideneacetone)dipalladium(0) (0.3 g, 0.28 mmol), tri-tert-butylphosphine (0.3 mL, 0.57 mmol), sodium tert-butoxide (0.8 g, 8.45 mmol), and 28 mL of toluene were added to a reaction vessel and stirred under reflux for 1 hour. After cooling the reaction mixture to room temperature, the solid was filtered and washed with ethyl acetate. The filtrate was distilled under reduced pressure and purified by column chromatography to obtain compound C-103 (1.7 g, yield: 53%).

| | MW | M.P. |
|---|---|---|
| C-103 | 563.3 | 171° C. |

### Example 7: Preparation of Compound C-104

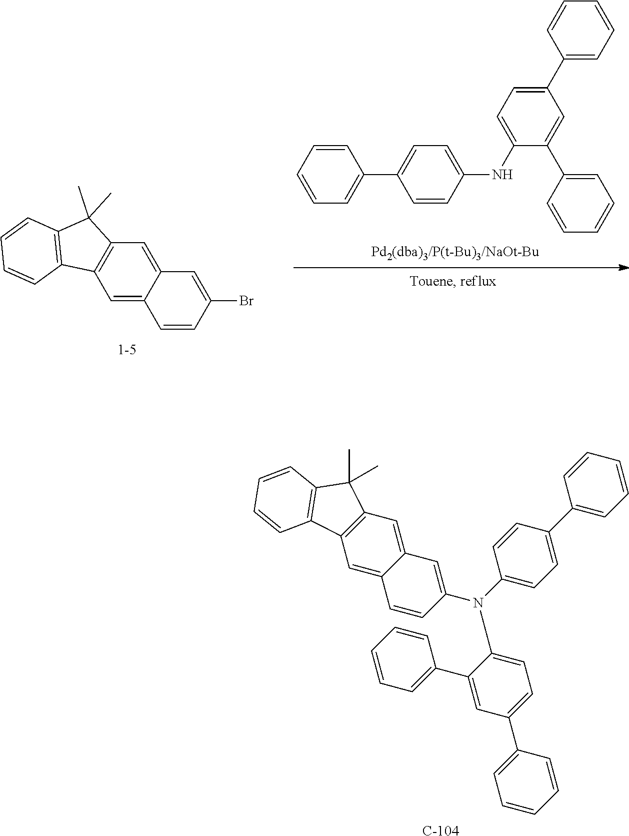 [SMILES: CC1(C)c2ccccc2-c2cc3ccc(Br)cc3cc21 ; c1ccc(-c2ccc(Nc3ccc(-c4ccccc4)cc3-c3ccccc3)cc2)cc1 ; CC1(C)c2ccccc2-c2cc3ccc(N(c4ccc(-c5ccccc5)cc4)c4ccc(-c5ccccc5)cc4-c4ccccc4)cc3cc21 ; "C-104"]

Compound 1-5 (4.7 g, 12.99 mmol), N-([1,1′-biphenyl]-4-yl)-{1,1′:3′,1″-terphenyl}-4′-amine (5.2 g, 12.99 mmol), tris(dibenzylideneacetone)dipalladium(0) (0.6 g, 0.65 mmol), tri-tert-butylphosphine (0.6 mL, 1.30 mmol), sodium tert-butoxide (1.9 g, 19.49 mmol), and 65 mL of toluene were added to a reaction vessel and stirred under reflux for 1 hour. After cooling the reaction mixture to room temperature, the solid was filtered and washed with ethyl acetate. The filtrate was distilled under reduced pressure and purified by column chromatography to obtain compound C-104 (2.7 g, yield: 33%).

| | MW | M.P. |
|---|---|---|
| C-104 | 563.3 | 226° C. |

### Example 8: Preparation of Compound C-27

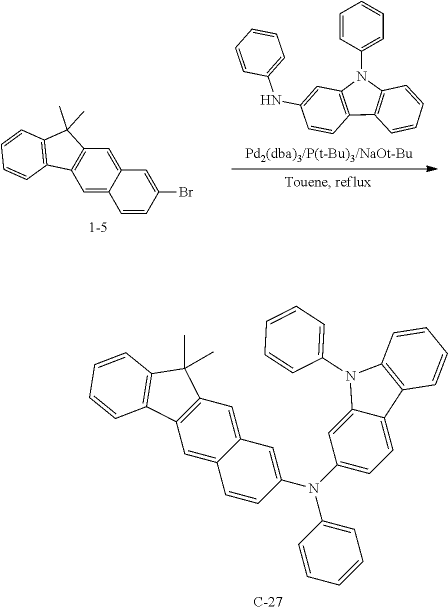

Compound 1-5 (3.0 g, 9.28 mmol), N,9-diphenyl-9H-carbazol-2-amine (3.1 g, 9.28 mmol), tris(dibenzylideneacetone)dipalladium(0) (0.4 g, 0.46 mmol), tri-tert-butylphosphine (0.5 mL, 0.93 mmol), sodium tert-butoxide (1.3 g, 13.92 mmol), and 47 mL of toluene were added to a reaction vessel and stirred under reflux for 1 hour. After cooling the reaction mixture to room temperature, the solid was filtered and washed with ethyl acetate. The filtrate was distilled under reduced pressure and purified by column chromatography to obtain compound C-27 (3.0 g, yield: 56%).

| | MW | M.P. |
|---|---|---|
| C-27 | 576.73 | 220° C. |

### Example 9: Preparation of Compound C-105

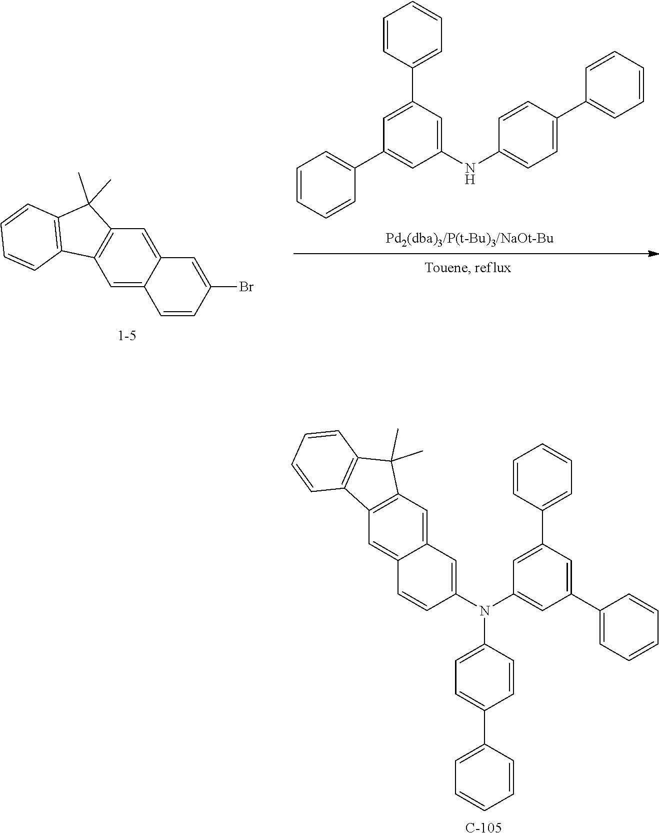

Compound 1-5 (5.62 g, 17.38 mmol). N-([1,1′-biphenyl]-4-yl)-{1,1′:3′,1″-terphenyl}-5′-amine (7.6 g, 19.12 mmol), tris(dibenzylideneacetone)dipalladium(0) (0.8 g, 0.87 mmol), tri-tert-butylphosphine (0.9 mL, 1.74 mmol), sodium tert-butoxide (2.5 g, 26.07 mmol), and 87 mL of toluene were added to a reaction vessel and stirred under reflux for 1 hour. After cooling the reaction mixture to room temperature, the solid was filtered and washed with ethyl acetate. The filtrate was distilled under reduced pressure and purified by column chromatography to obtain compound C-105 (7.5 g, yield: 67%).

| | MW | M.P. |
|---|---|---|
| C-105 | 639.8 | 171° C. |

### Example 10: Preparation of Compound C-106

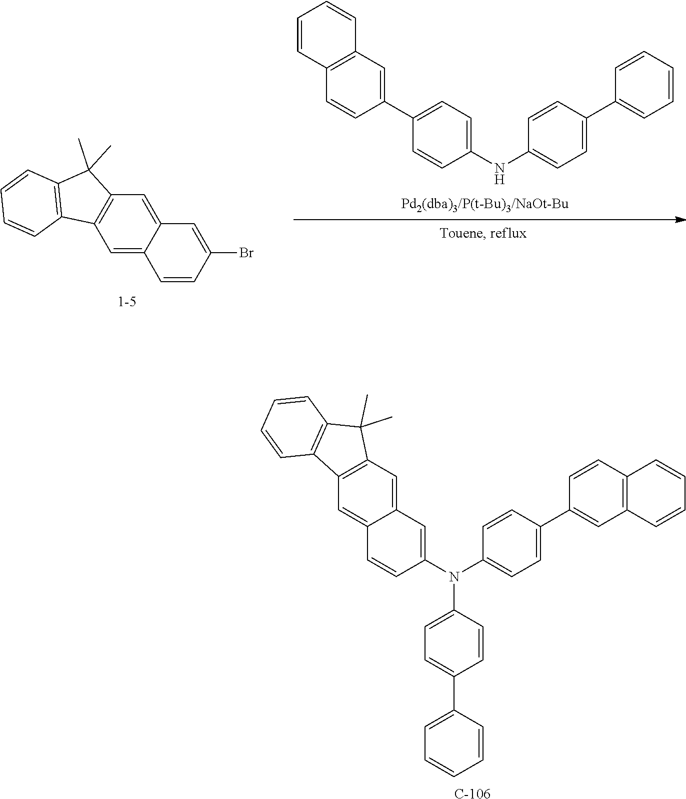

Compound 1-5 (4.0 g, 12.38 mmol), N-(4-[naphthalen-2-yl]phenyl)-[1,1′-biphenyl]-4-amine (5.05 g, 13.61 mmol), tris(dibenzylideneacetone)dipalladium(0) (0.6 g, 0.62 mmol), tri-tert-butylphosphine (0.6 mL, 1.24 mmol), sodium tert-butoxide (1.8 g, 18.57 mmol), and 62 mL of toluene were added to a reaction vessel and stirred under reflux for 1 hour. After cooling the reaction mixture to room temperature, the solid was filtered and washed with ethyl acetate. The filtrate was distilled under reduced pressure and purified by column chromatography to obtain compound C-106 (3.8 g, yield: 50%).

| | MW | M.P. |
|---|---|---|
| C-106 | 613.8 | 235° C. |

### Example 11: Preparation of Compound C-107

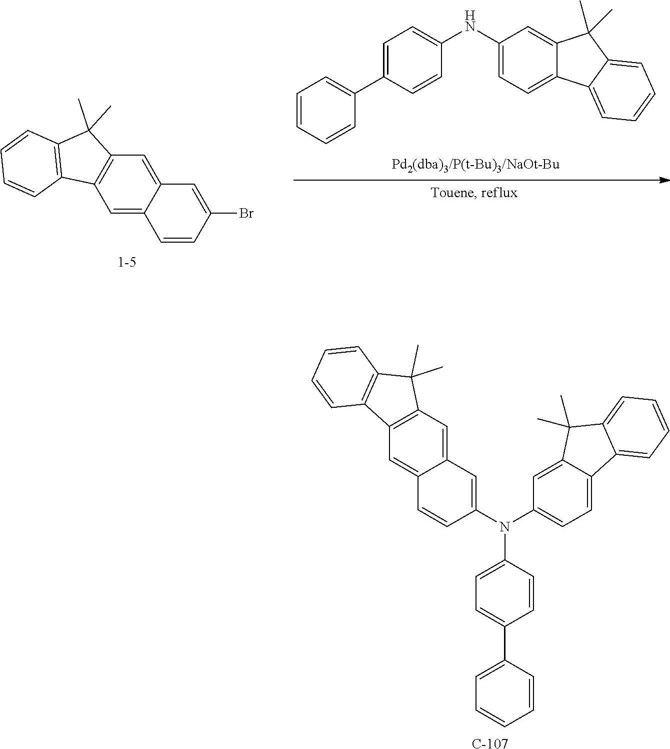

Compound 1-5 (3.5 g, 10.82 mmol), N-([1,1′-biphenyl]-4-yl)-9,9′-dimethyl-9H-fluoren-2-amine (4.3 g, 11.91 mmol), tris(dibenzylideneacetone)dipalladium(0) (0.5 g, 0.54 mmol), tri-tert-butylphosphine (0.55 mL, 1.08 mmol), sodium tert-butoxide (1.6 g, 16.23 mmol), and 54 mL of toluene were added to a reaction vessel and stirred under reflux for 1 hour. After cooling the reaction mixture to room temperature, the solid was filtered and washed with ethyl acetate. The filtrate was distilled under reduced pressure and purified by column chromatography to obtain compound C-107 (5.4 g, yield: 82%).

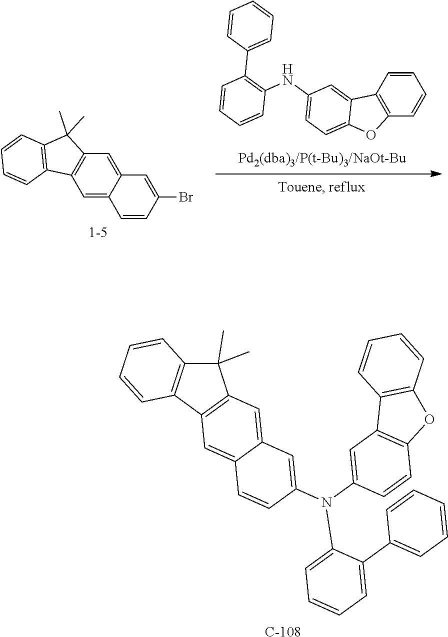 [SMILES: CC1(C)c2ccccc2-c2cc3ccc(Br)cc3cc21 ; c1ccc(-c2ccccc2Nc2ccc3oc4ccccc4c3c2)cc1 ; CC1(C)c2ccccc2-c2cc3ccc(N(c4ccc5oc6ccccc6c5c4)c4ccccc4-c4ccccc4)cc3cc21 ; "C-108"]

### Example 12: Preparation of Compound C-108

| | MW | M.P. |
|---|---|---|
| C-107 | 603.8 | 240° C. |

Compound 1-5 (3.5 g, 10.82 mmol), N-([1,1′-biphenyl]-2-yl)dibenzo[b,d]furan-2-amine (4.0 g, 11.91 mmol), tris(dibenzylideneacetone)dipalladium(0) (0.5 g, 0.54 mmol), tri-tert-butylphosphine (0.55 mL, 1.08 mmol), sodium tert-butoxide (1.6 g, 16.23 mmol), and 54 mL of toluene were added to a reaction vessel and stirred under reflux for 1 hour. After cooling the reaction mixture to room temperature, the solid was filtered and washed with ethyl acetate. The filtrate was distilled under reduced pressure and purified by column chromatography to obtain compound C-108 (2.8 g, yield: 45%).

| | MW | M.P. |
|---|---|---|
| C-108 | 577.7 | 158° C. |

### Example 13: Preparation of Compound C-43

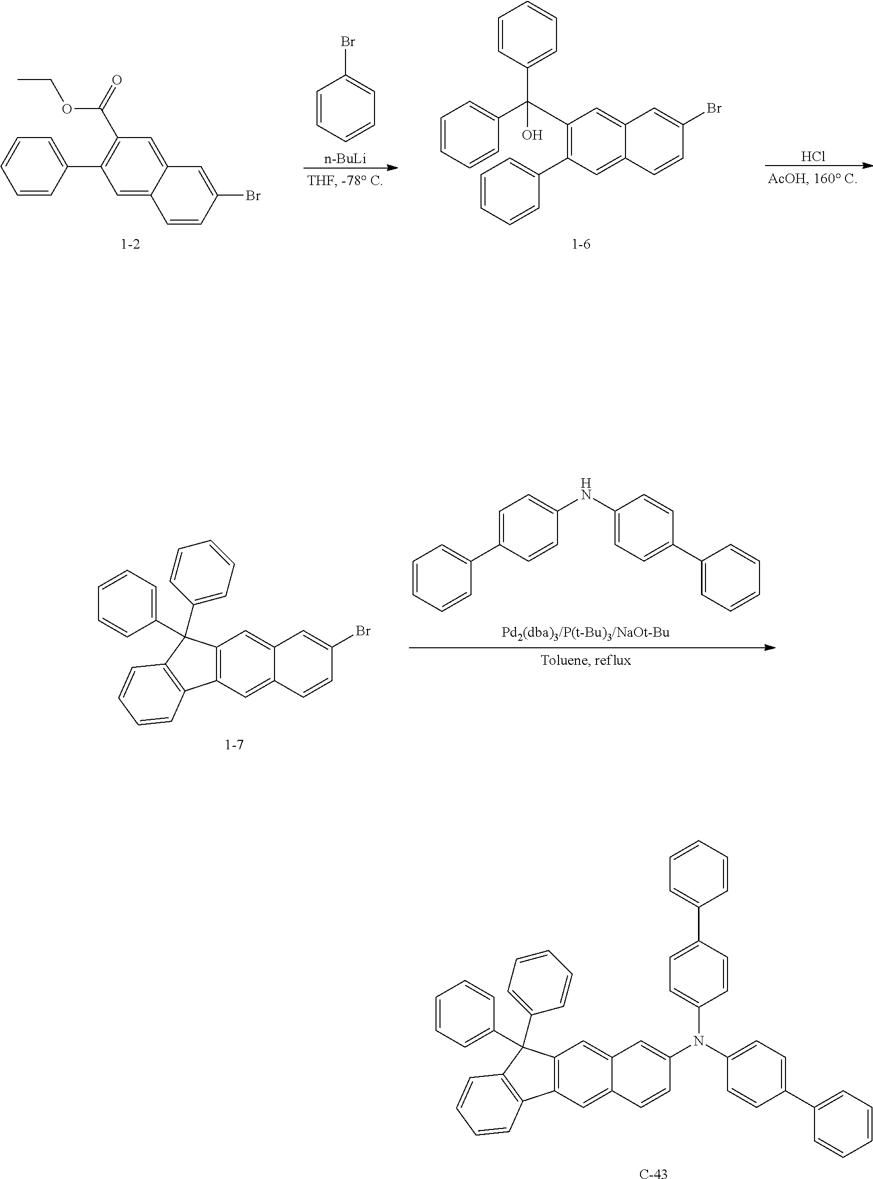

1) Synthesis of Compound 1-6

Bromobenzene (14 mL, 132.6 mmol) and 221 mL of tetrahydrofuran were added to a reaction vessel and stirred at −78° C. After 10 minutes, n-butyllithium (53 mL, 132.6 mmol) was slowly added and stirred. After 1 hour, compound 1-2 (15.7 g, 44.2 mmol) was added and stirred at room temperature for 18 hours. After the reaction was completed, the mixture was washed with 200 mL of water and 200 mL of ammonium chloride, extracted with ethyl acetate, dried with magnesium sulfate, and then the solvent was removed by a rotary evaporator to obtain compound 1-6 (24.8 g, yield: 120%).

2) Synthesis of Compound 1-7

Compound 1-6 (24.8 g, 53.4 mmol), hydrochloric acid (22.3 mL), and acetic acid (223 mL) were added to a reaction vessel and stirred at 160° C. After 18 hours, the resulting solid was filtered under reduced pressure, washed with water, aqueous sodium hydroxide solution, methanol, and hexane, and then purified by column chromatography to obtain compound 1-7 (12.0 g, yield: 50%).

2) Synthesis of Compound C-43

Compound 1-7 (4.0 g, 8.95 mmol), bis(4-biphenyl)amine (2.6 g, 8.14 mmol), tris(dibenzylideneacetone)dipalladium(0) (0.37 g, 0.41 mmol), tri-tert-butylphosphine (0.4 mL, 0.81 mmol), sodium tert-butoxide (1.2 g, 12.2 mmol), and 40 mL of toluene were added to a reaction vessel and stirred under reflux for 1 hour. After cooling the reaction mixture to room temperature, the solid was filtered and washed with ethyl acetate. The filtrate was distilled under reduced pressure and purified by column chromatography to obtain compound C-43 (4.3 g, yield: 70%).

| | MW | M.P. |
|---|---|---|
| C-43 | 687.3 | 273° C. |

### Example 14: Preparation of Compound C-47

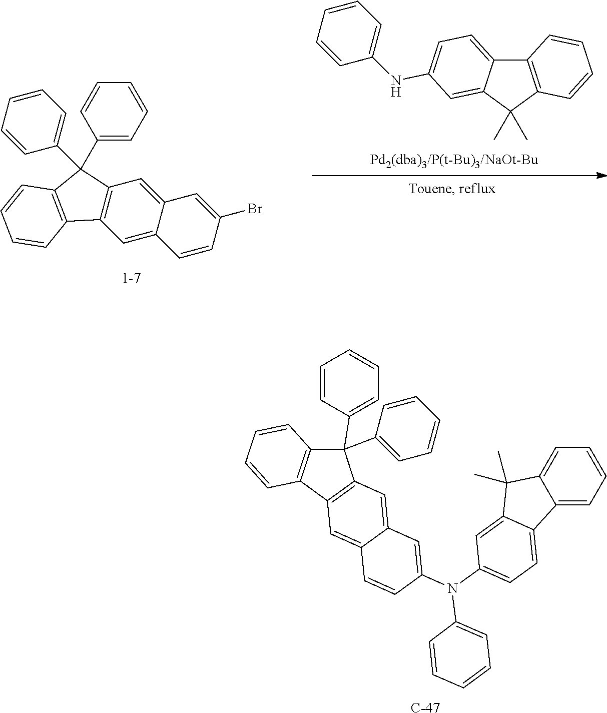

Compound 1-7 (4.0 g, 8.95 mmol), 9,9-dimethyl-N-phenyl-9H-fluoren-2-amine (2.3 g, 8.14 mmol), tris(dibenzylideneacetone)dipalladium(0) (0.4 g, 0.41 mmol), tri-tert-butylphosphine (0.4 mL, 0.81 mmol), sodium tert-butoxide (1.2 g, 12.21 mmol), and 40 mL of toluene were added to a reaction vessel and stirred under reflux for 1 hour. After cooling the reaction mixture to room temperature, the solid was filtered and washed with ethyl acetate. The filtrate was distilled under reduced pressure and purified by column chromatography to obtain compound C-47 (3.9 g, yield: 74%).

| | MW | M.P. |
|---|---|---|
| C-47 | 651.84 | 320° C. |

### Example 15: Preparation of Compound C-101

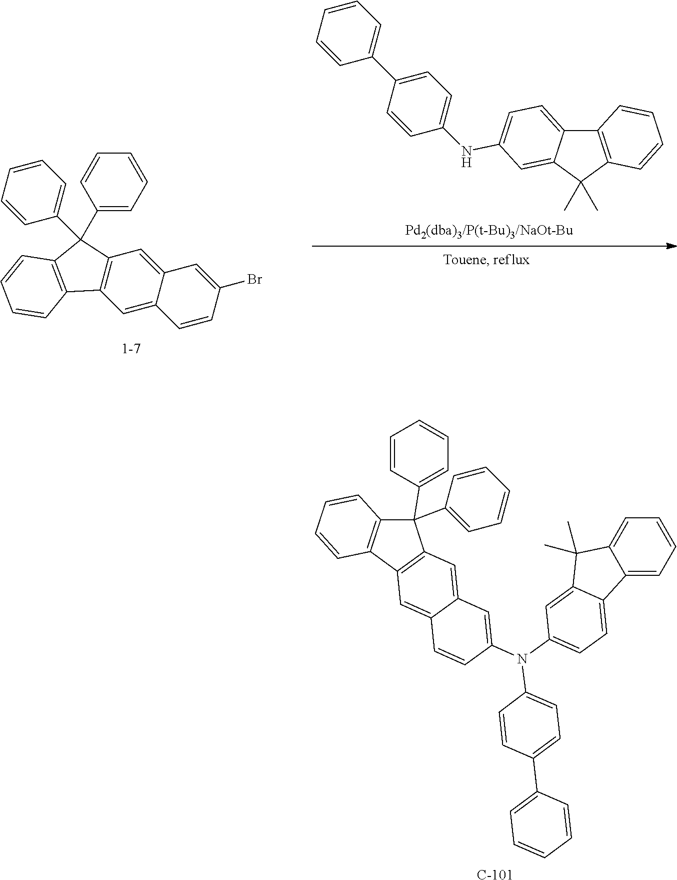

Compound 1-7 (4.0 g, 8.95 mmol), N-([1,1′-biphenyl]-4-yl)-9,9′-dimethyl-9H-fluoren-2-amine (3.2 g, 8.95 mmol), tris(dibenzylideneacetone)dipalladium(0) (0.4 g, 0.45 mmol), tri-tert-butylphosphine (0.4 mL, 0.90 mmol), sodium tert-butoxide (1.3 g, 13.43 mmol), and 45 mL of toluene were added to a reaction vessel and stirred under reflux for 1 hour. After cooling the reaction mixture to room temperature, the solid was filtered and washed with ethyl acetate. The filtrate was distilled under reduced pressure and purified by column chromatography to obtain compound C-101 (3.9 g, yield: 74%).

| | MW | M.P. |
|---|---|---|
| C-101 | 727.93 | 324° C. |

### Example 16: Preparation of Compound C-53

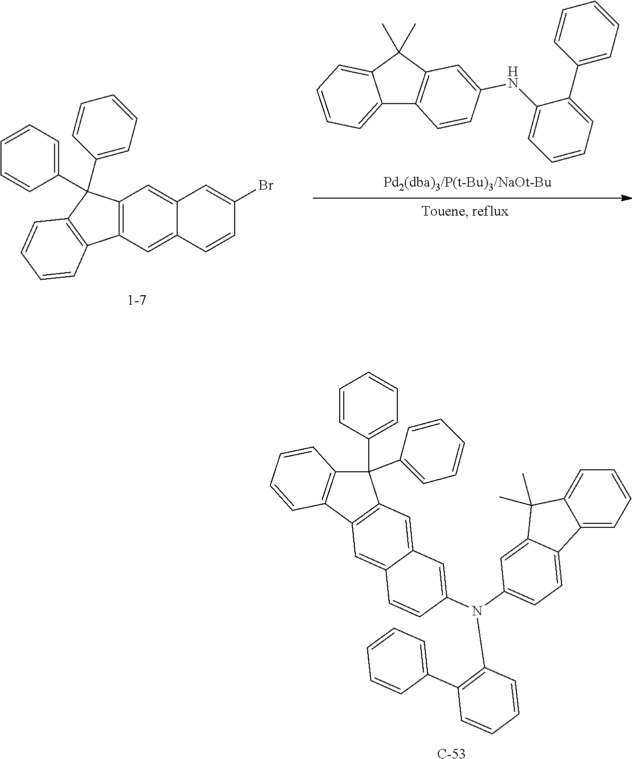

Compound 1-7 (6.0 g, 13.45 mmol), N-([1,1′-biphenyl]-2-yl)-9,9′-dimethyl-9H-fluoren-2-amine (5.8 g, 16.14 mmol), tris(dibenzylideneacetone)dipalladium(0) (0.62 g, 0.67 mmol), tri-tert-butylphosphine (0.66 mL, 1.35 mmol), sodium tert-butoxide (2.6 g, 26.9 mmol), and 70 mL of toluene were added to a reaction vessel and stirred under reflux for 4 hours. After cooling the reaction mixture to room temperature, the solid was filtered and washed with ethyl acetate. The filtrate was distilled under reduced pressure and purified by column chromatography to obtain compound C-53 (5.4 g, yield: 55%).

| | MW | M.P. |
|---|---|---|
| C-53 | 727.95 | 173° C. |

### Example 17: Preparation of Compound C-102

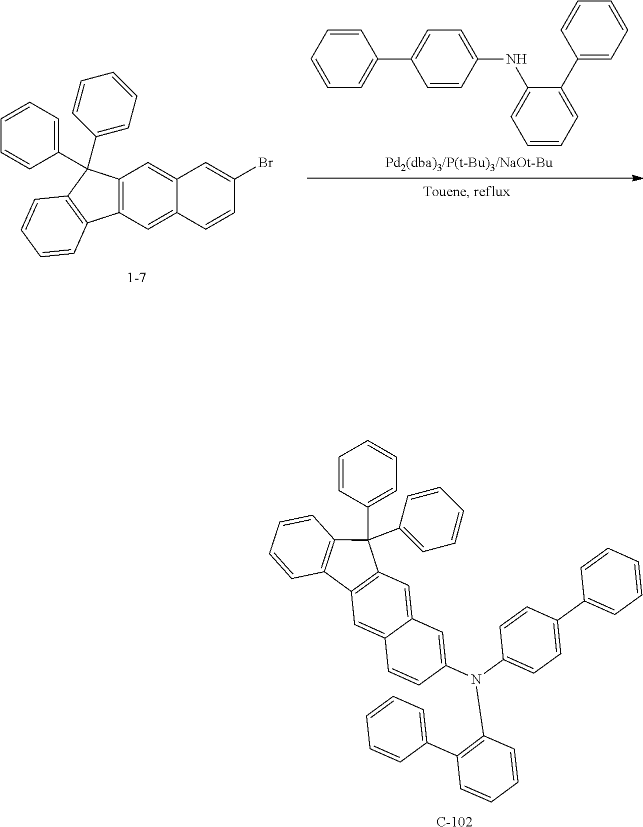

Compound 1-7 (6.0 g, 13.45 mmol), N-([1,1′-biphenyl]-4-yl)-[1,1′-biphenyl]-2-amine (5.2 g, 16.14 mmol), tris(dibenzylideneacetone)dipalladium(0) (0.62 g, 0.67 mmol), tri-tert-butylphosphine (0.66 mL, 1.35 mmol), sodium tert-butoxide (2.6 g, 26.9 mmol), and 70 mL of toluene were added to a reaction vessel and stirred under reflux for 4 hours. After cooling the reaction mixture to room temperature, the solid was filtered and washed with ethyl acetate. The filtrate was distilled under reduced pressure and purified by column chromatography to obtain compound C-102 (5.0 g, yield: 54%).

| | MW | M.P. |
|---|---|---|
| C-102 | 687.89 | 230° C. |

### Example 18: Preparation of Compound C-109

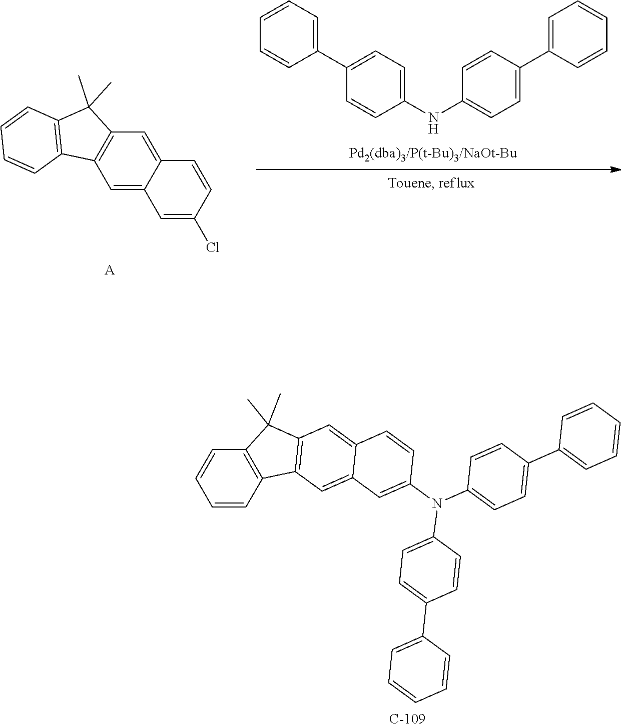

Compound A (3.0 g, 9.28 mmol), di([1,1′-biphenyl]-4-yl)amine (3.3 g, 10.21 mmol), tris(dibenzylideneacetone)dipalladium(0) (0.35 g, 0.39 mmol), tri-tert-butylphosphine (0.4 mL, 0.77 mmol), sodium tert-butoxide (1.1 g, 11.6 mmol), and 40 mL of toluene were added to a reaction vessel and stirred under reflux for 1 hour. After cooling the reaction mixture to room temperature, the solid was filtered and washed with ethyl acetate. The filtrate was distilled under reduced pressure and purified by column chromatography to obtain compound C-109 (1.5 g, yield: 26%).

| | MW | M.P. |
|---|---|---|
| C-109 | 563.30 | 254° C. |

Hereinafter, a method of manufacturing an organic electroluminescent device comprising the compound of the present disclosure and light-emitting characteristics will be described for a detailed understanding of the present disclosure. The following examples are only to describe the characteristics of the OLED according to the present disclosure for a detailed understanding of the present disclosure, but the present disclosure is not limited thereto.

### Device Example 1: Producing an OLED Using a Compound According to the Present Disclosure

An OLED was produced by using an organic electroluminescent compound according to the present disclosure. First of all, a transparent electrode indium tin oxide (ITO) thin film (10 Ω/sq) obtained from a glass for an OLED (GEOMATEC CO., LTD., Japan) was subjected to an ultrasonic washing with acetone, ethanol, and distilled water, sequentially, and then was stored in isopropanol. After evacuating until the degree of vacuum in the chamber reached 10⁻⁶ torr, the ITO substrate was mounted on a substrate holder of the vacuum vapor deposition apparatus. Compound HT-1 was introduced into a cell of the vacuum vapor deposition apparatus, and compound HI-1 was introduced into another cell. The two materials were evaporated at different rates to deposit a hole injection layer with a thickness of 10 nm by doping compound HI-1 in an amount of 3 wt % based to the total amount of compound HT-1 and compound HI-1. Subsequently, compound HT-1 was introduced into a cell of the vacuum vapor deposition apparatus and was evaporated by applying an electric current to the cell, thereby depositing a first hole transport layer with a thickness of 90 nm. Subsequently, compound C-19 according to the present disclosure was introduced into another cell of the vacuum vapor deposition apparatus and was evaporated by applying an electric current to the cell, thereby depositing a second hole transport layer with a thickness of 60 nm on the first hole transport layer. After forming the hole injection layer and the hole transport layers, a light-emitting layer was formed thereon as follows: Compound RH-1 was introduced into a cell of the vacuum vapor deposition apparatus as a host of the light-emitting layer, and compound RD was introduced into another cell as a dopant. The two materials were evaporated to deposit a light-emitting layer with a thickness of 40 nm on the second hole transport layer by doping the dopant in an amount of 2 wt % based on the total amount of the host and dopant. Subsequently, compound ET and compound EI in two different cells were evaporated at a rate of 1:1 to deposit an electron transport layer with a thickness of 35 nm on the light-emitting layer. After depositing compound EI to a thickness of 2 nm as an electron injection layer, an Al cathode was deposited to a thickness of 80 nm by using another vacuum vapor deposition apparatus to produce an OLED.

### Comparative Example 1: Producing an OLED Using a Conventional Compound

An OLED was produced in the same manner as in Device Example 1, except that compound A instead of compound C-19 was used.

Method of Measuring Refractive Index

The refractive index of the compound used in the second hole transport layer in the Device Example and the Comparative Example was measured by the following method. After putting the compound, for which the refractive index is to be measured, into a cell of the vacuum vapor deposition apparatus and evacuating it until the degree of vacuum in the chamber reached 10⁻⁶ torr, an electric current was applied to the cell to evaporate, thereby producing a specimen with a thickness of 30 nm on the silicon wafer substrate. The refractive index was measured using an Ellipsometer. Specifically, a refractive index based on 620 nm at an incidence angle of 60° was used by utilizing UVISEL from HORIBA at a wavelength of 350 nm to 800 nm.

The measurement results of driving voltage, power efficiency, and color coordinate based on a luminance of 1,000 cd/m² of the OLED produced in Device Example 1 and Comparative Example 1, and the measurement results of refractive index of the compound used in Device Example 1 and Comparative Example 1 are shown in Table 1 below.

TABLE 1

| | Second Hole | | | | | | |
|---|---|---|---|---|---|---|---|
| | Transport | | Driving | Efficiency | Color Coordinate | | Refractive |
| | Layer | Host | Voltage [V] | [lm/W] | x | y | Index |
| Device | C-19 | RH-1 | 2.9 | 30.5 | 0.659 | 0.341 | 1.78 |
| Example 1 | | | | | | | |
| Comparative | A | RH-1 | 3.1 | 28.7 | 0.666 | 0.334 | 1.71 |
| Example 1 | | | | | | | |

### Device Examples 2-1 to 2-3: Producing an OLED Using a Compound According to the Present Disclosure

An OLED was produced in the same manner as in Device Example 1, except that compound shown in Table 2 below was used instead of compound C-19 as the compound for the second hole transport layer, and compound RH-2 was used instead of compound RH-1 as the host of the light-emitting layer.

### Comparative Examples 2-1 and 2-2: Producing an OLED Using a Conventional Compound

An OLED was produced in the same manner as in Device Example 1, except that compound shown in Table 2 below was used instead of compound C-19 as the compound for the second hole transport layer, and compound RH-2 was used instead of compound RH-1 as the host of the light-emitting layer.

The measurement results of driving voltage, power efficiency, and color coordinate based on a luminance of 1,000 cd/m² of the OLED produced in Device Examples 2-1 to 2-3 and Comparative Example 2-1 and 2-2, and the measurement results of refractive index of the compound used in Device Examples 2-1 to 2-3 and Comparative Example 2-1 and 2-2 are shown in Table 2 below.

TABLE 2

| | Second Hole | | | | | | |
|---|---|---|---|---|---|---|---|
| | Transport | | Driving | Efficiency | Color Coordinate | | Refractive |
| | Layer | Host | Voltage [V] | [lm/W] | x | y | Index |
| Device | C-18 | RH-2 | 3.0 | 32.8 | 0.660 | 0.340 | 1.81 |
| Example 2-1 | | | | | | | |
| Device | C-101 | RH-2 | 3.1 | 32.1 | 0.660 | 0.339 | 1.79 |
| Example 2-2 | | | | | | | |
| Device | C-109 | RH-2 | 3.0 | 32.6 | 0.661 | 0.338 | 1.80 |
| Example 2-3 | | | | | | | |
| Comparative | A | RH-2 | 3.1 | 30.8 | 0.658 | 0.342 | 1.71 |
| Example 2-1 | | | | | | | |
| Comparative | B | RH-2 | 3.3 | 30.8 | 0.660 | 0.340 | 1.72 |
| Example 2-2 | | | | | | | |

According to the results above, the organic electroluminescent compound according to the present disclosure has a higher refractive index than that of the conventional compound, and thus an effect of reducing the thickness of the device is expected. In addition, it was confirmed that the Device Examples according to the present disclosure can provide a deep red organic electroluminescent device with improved driving voltage and power efficiency even when a hole transport layer having the same thickness as compared to the Comparative Examples is used. This is understood that the larger the refractive index of the material, the slower the speed of light travel, and the number of molecules in the unit volume increases, thereby improving the inter-molecular electron hopping ability, thereby lowering the driving voltage of the organic electroluminescent device, resulting in the effect of saving power consumption.

The compounds used in the Device Examples and Comparative Examples are shown in Table 3 below.

TABLE 3

| | |
|---|---|
| Hole Injection Layer/ First Hole Transport Layer | 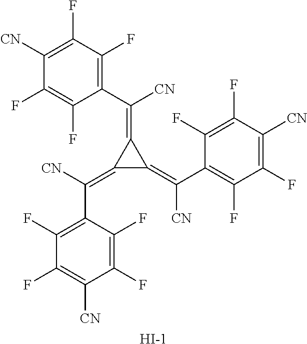 |
| | 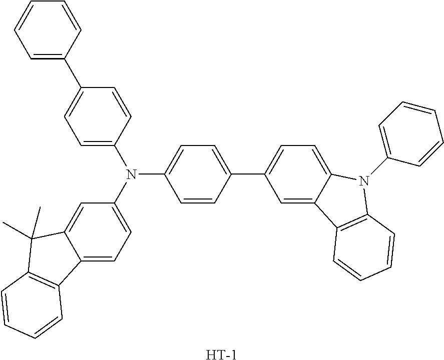 |
| Second Hole Transport Layer | 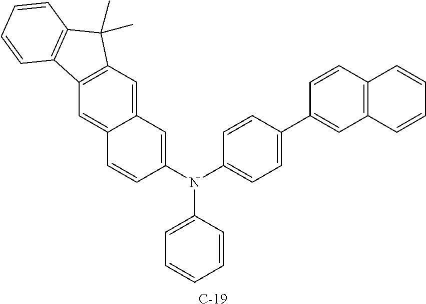 |
| | 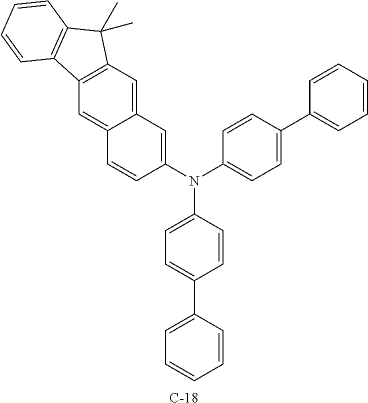 |
| | 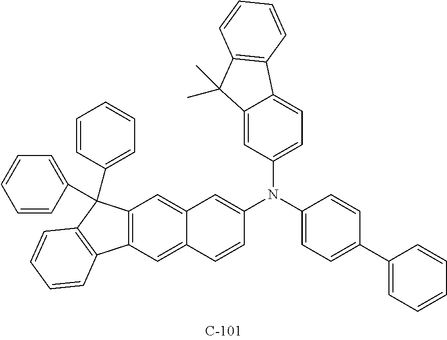 [SMILES: CC1(C)c2ccccc2-c2ccc(N(c3ccc(-c4ccccc4)cc3)c3ccc4cc5c(cc4c3)C(c3ccccc3)(c3ccccc3)c3ccccc3-5)cc21] |
| | 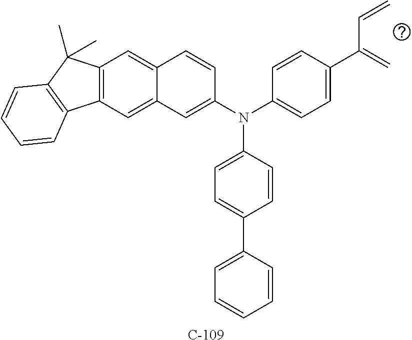 |
| | 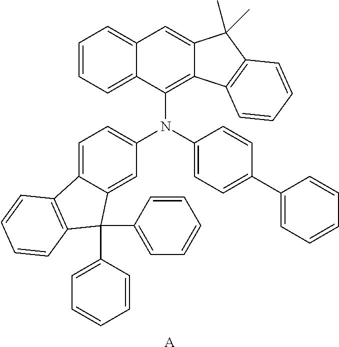 |
| | 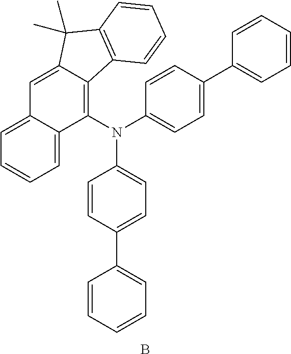 |
| Light- Emitting Layer | 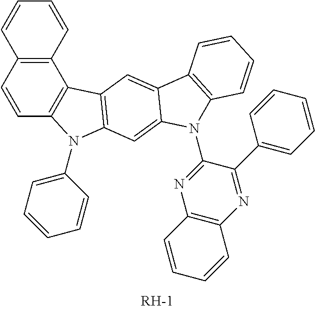 [SMILES: c1ccc(-c2nc3ccccc3nc2-n2c3ccccc3c3cc4c5c6ccccc6ccc5n(-c5ccccc5)c4cc32)cc1] |
| | 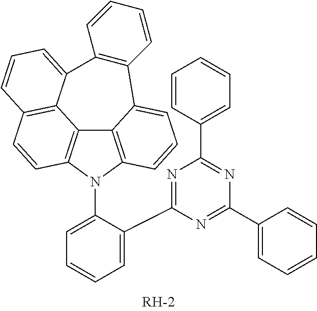 [SMILES: c1ccc(-c2nc(-c3ccccc3)nc(-c3ccccc3-n3c4cccc5c4c4c6c(cccc6ccc43)-c3ccccc3-5)n2)cc1] |
| | 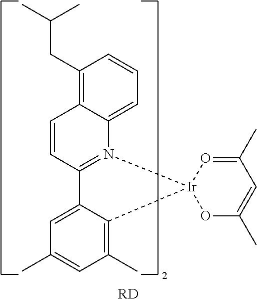 |
| Electron Transport Layer/ Electron Injection Layer | 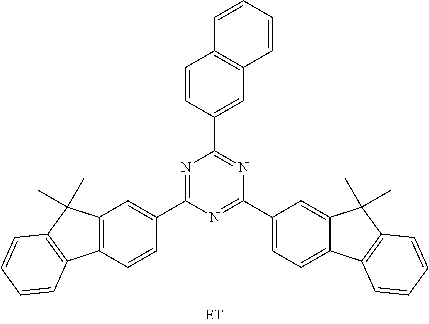 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 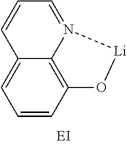 |

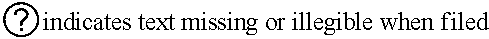

1. An organic electroluminescent compound represented by the following formula 1: 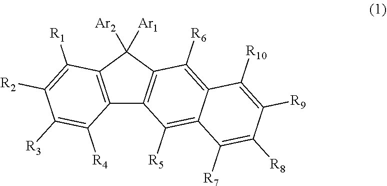
wherein
Ar₁ and Ar₂ each independently represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, or a substituted or unsubstituted (3- to 30-membered)heteroaryl, or Ar₁ and Ar₂ may be linked to each other to form a fused ring;
R₁ to R₆ each independently represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsiyl, or a substituted or unsubstituted tri(C6-C30)arylsilyl;
R₇ to R₁₀ each independently represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or 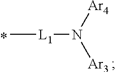
with a proviso that at least one of R₇ to R₁₀ is 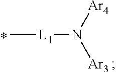
L₁ represents a single bond, a substituted or unsubstituted (C6-C30)arylene, or a substituted or unsubstituted (3- to 30-membered)heteroarylene;
Ar₃ and Ar₄ each independently represent hydrogen, deuterium, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, or a substituted or unsubstituted (3- to 30-membered)heteroaryl, or Ar₃ and Ar₄ may be linked to each other to form a fused ring; and
* represents the position linked to benzofluorene.

2. The organic electroluminescent compound according to claim 1, wherein formula 1 is represented by at least one of the following formulae 1-1 to 1-4: 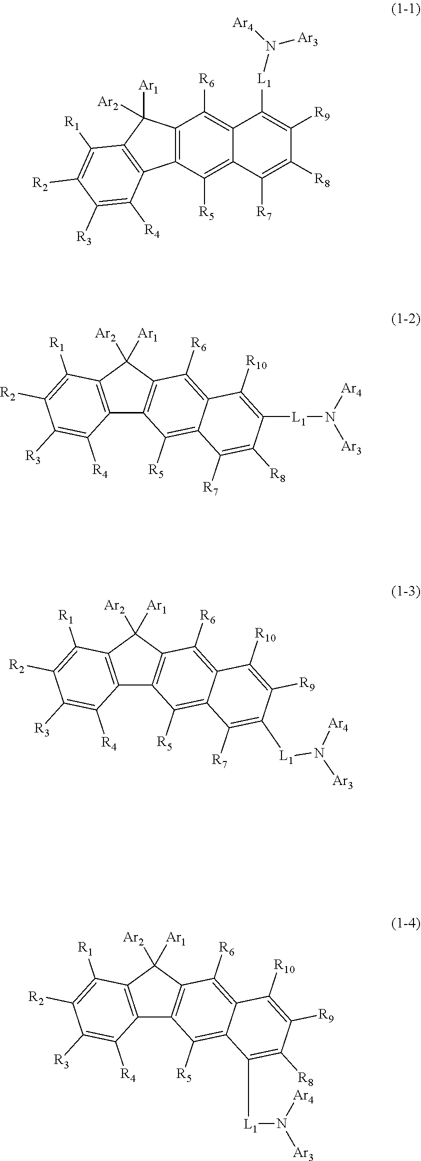
wherein
Ar₁ to Ar₄, R₁ to R₆, and L₁ are as defined in formula 1; and
R₇ to R₁₀ each independently represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted tri(C6-C30)arylsilyl.

3. The organic electroluminescent compound according to claim 1, wherein the substituents of the substituted alkyl, the substituted aryl, the substituted arylene, the substituted heteroaryl, the substituted heteroarylene, the substituted cycloalkyl, the substituted heterocycloalkyl, the substituted trialkylsilyl, the substituted dialkylarylsilyl, the substituted alkyldiarylsilyl, and the substituted triarylsilyl, each independently, are at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with at least one of a (C1-C30)alkyl(s), a (3- to 30-membered)heteroaryl(s) and a di(C6-C30)arylamino(s); a tri(C1-C30)alkylsilyl; a tri(C6-C30)arylsilyl; a di(C1-C30)alkyl(C6-C30)arysilyl; a (C1-C30)alkyldi(C6-C30)arylsilyl; an amino; a mono- or di-(C1-C30)alkylamino; a mono- or di-(C2-C30)alkenylamino; a mono- or di-(C6-C30)arylamino; a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a di(C6-C30)arylboronyl; a (C6-C30)arylphosphine; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

4. The organic electroluminescent compound according to claim 1, wherein the compound represented by formula 1 is at least one selected from the following compounds:

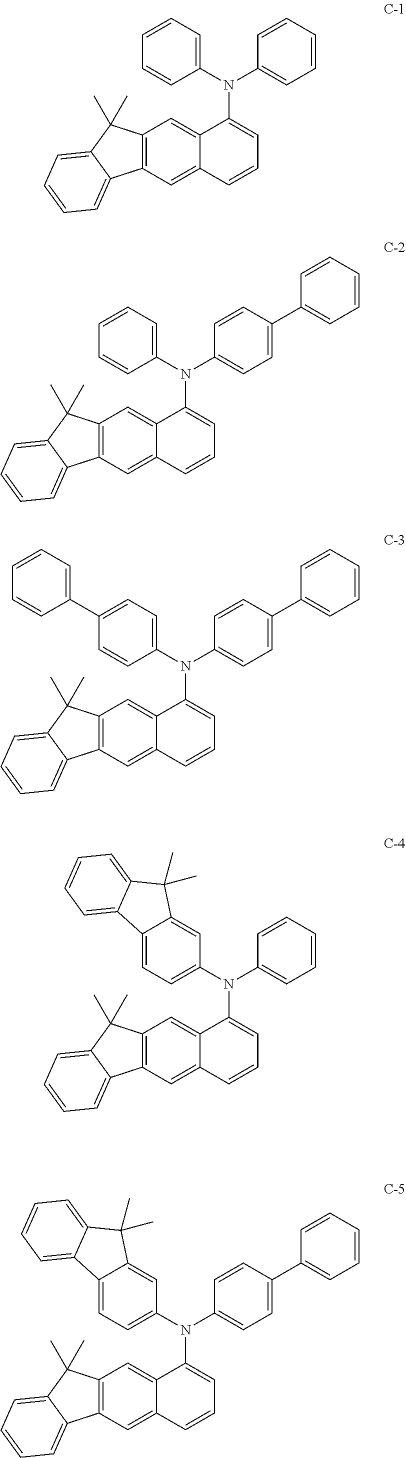 [SMILES: CC1(C)c2ccccc2-c2cc3cccc(N(c4ccccc4)c4ccccc4)c3cc21 ; CC1(C)c2ccccc2-c2cc3cccc(N(c4ccccc4)c4ccc(-c5ccccc5)cc4)c3cc21 ; CC1(C)c2ccccc2-c2cc3cccc(N(c4ccc(-c5ccccc5)cc4)c4ccc(-c5ccccc5)cc4)c3cc21 ; CC1(C)c2ccccc2-c2ccc(N(c3ccccc3)c3cccc4cc5c(cc34)C(C)(C)c3ccccc3-5)cc21 ; CC1(C)c2ccccc2-c2ccc(N(c3ccc(-c4ccccc4)cc3)c3cccc4cc5c(cc34)C(C)(C)c3ccccc3-5)cc21]

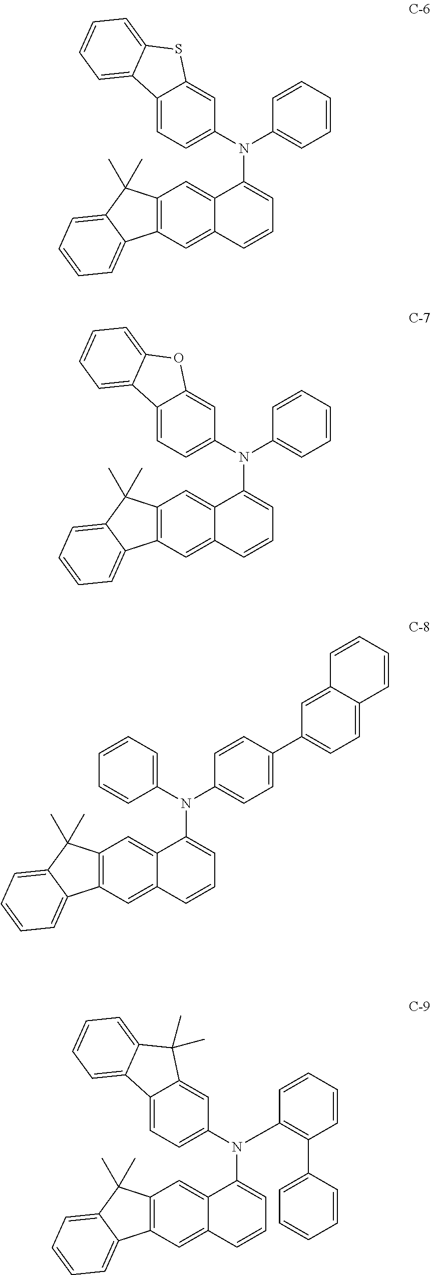 [SMILES: CC1(C)c2ccccc2-c2cc3cccc(N(c4ccccc4)c4ccc5c(c4)sc4ccccc45)c3cc21 ; CC1(C)c2ccccc2-c2cc3cccc(N(c4ccccc4)c4ccc5c(c4)oc4ccccc45)c3cc21 ; CC1(C)c2ccccc2-c2cc3cccc(N(c4ccccc4)c4ccc(-c5ccc6ccccc6c5)cc4)c3cc21 ; CC1(C)c2ccccc2-c2ccc(N(c3ccccc3-c3ccccc3)c3cccc4cc5c(cc34)C(C)(C)c3ccccc3-5)cc21]

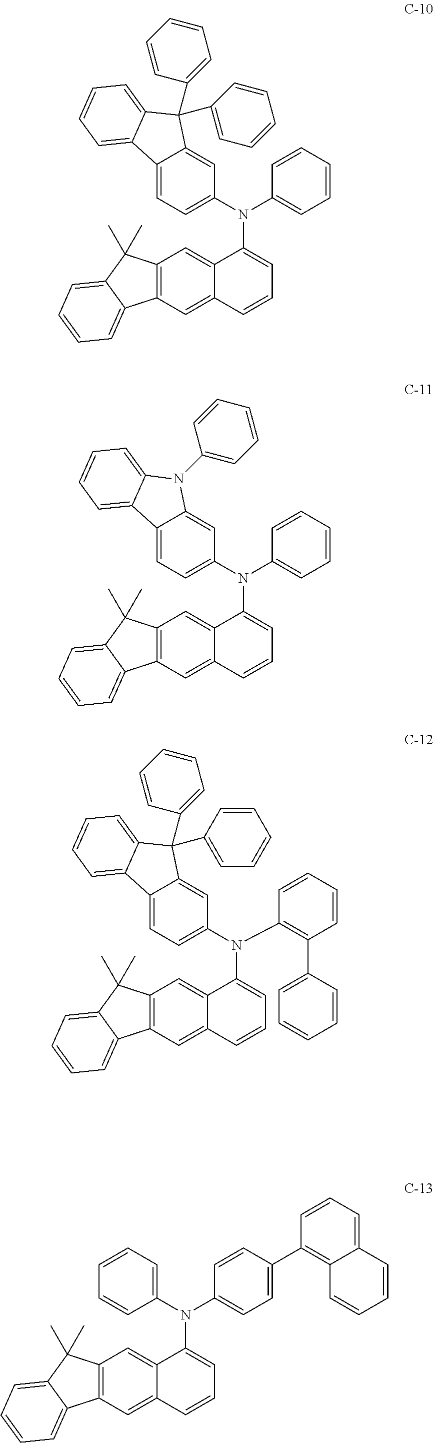 [SMILES: CC1(C)c2ccccc2-c2cc3cccc(N(c4ccccc4)c4ccc5c(c4)C(c4ccccc4)(c4ccccc4)c4ccccc4-5)c3cc21 ; CC1(C)c2ccccc2-c2cc3cccc(N(c4ccccc4)c4ccc5c6ccccc6n(-c6ccccc6)c5c4)c3cc21 ; CC1(C)c2ccccc2-c2cc3cccc(N(c4ccc5c(c4)C(c4ccccc4)(c4ccccc4)c4ccccc4-5)c4ccccc4-c4ccccc4)c3cc21 ; CC1(C)c2ccccc2-c2cc3cccc(N(c4ccccc4)c4ccc(-c5cccc6ccccc56)cc4)c3cc21]

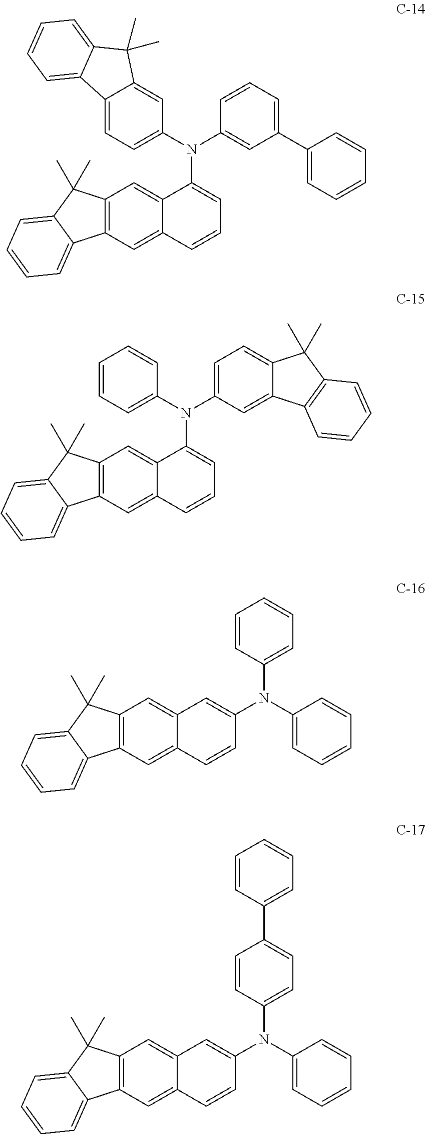 [SMILES: CC1(C)c2ccccc2-c2ccc(N(c3cccc(-c4ccccc4)c3)c3cccc4cc5c(cc34)C(C)(C)c3ccccc3-5)cc21 ; CC1(C)c2ccccc2-c2cc(N(c3ccccc3)c3cccc4cc5c(cc34)C(C)(C)c3ccccc3-5)ccc21 ; CC1(C)c2ccccc2-c2cc3ccc(N(c4ccccc4)c4ccccc4)cc3cc21 ; CC1(C)c2ccccc2-c2cc3ccc(N(c4ccccc4)c4ccc(-c5ccccc5)cc4)cc3cc21]

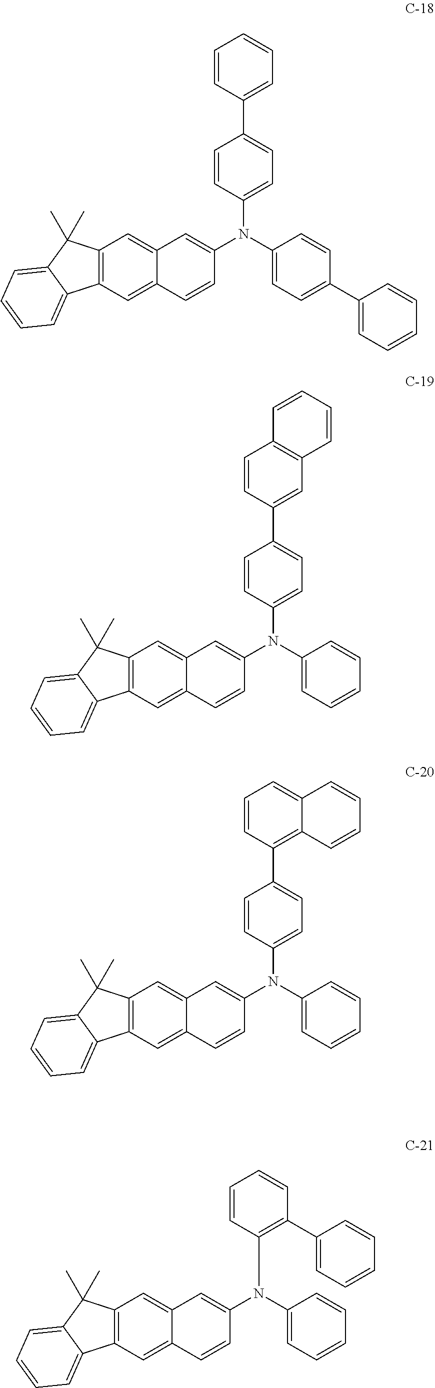 [SMILES: CC1(C)c2ccccc2-c2cc3ccc(N(c4ccc(-c5ccccc5)cc4)c4ccc(-c5ccccc5)cc4)cc3cc21 ; CC1(C)c2ccccc2-c2cc3ccc(N(c4ccccc4)c4ccc(-c5ccc6ccccc6c5)cc4)cc3cc21 ; CC1(C)c2ccccc2-c2cc3ccc(N(c4ccccc4)c4ccc(-c5cccc6ccccc56)cc4)cc3cc21 ; CC1(C)c2ccccc2-c2cc3ccc(N(c4ccccc4)c4ccccc4-c4ccccc4)cc3cc21]

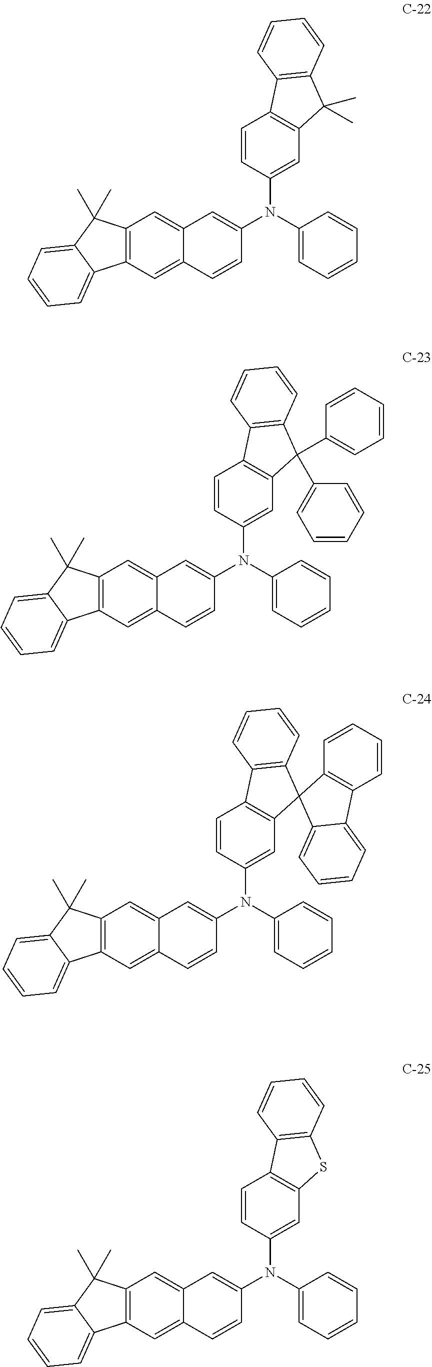 [SMILES: CC1(C)c2ccccc2-c2ccc(N(c3ccccc3)c3ccc4cc5c(cc4c3)C(C)(C)c3ccccc3-5)cc21 ; CC1(C)c2ccccc2-c2cc3ccc(N(c4ccccc4)c4ccc5c(c4)C(c4ccccc4)(c4ccccc4)c4ccccc4-5)cc3cc21 ; CC1(C)c2ccccc2-c2cc3ccc(N(c4ccccc4)c4ccc5c(c4)C4(c6ccccc6-c6ccccc64)c4ccccc4-5)cc3cc21 ; CC1(C)c2ccccc2-c2cc3ccc(N(c4ccccc4)c4ccc5c(c4)sc4ccccc45)cc3cc21]

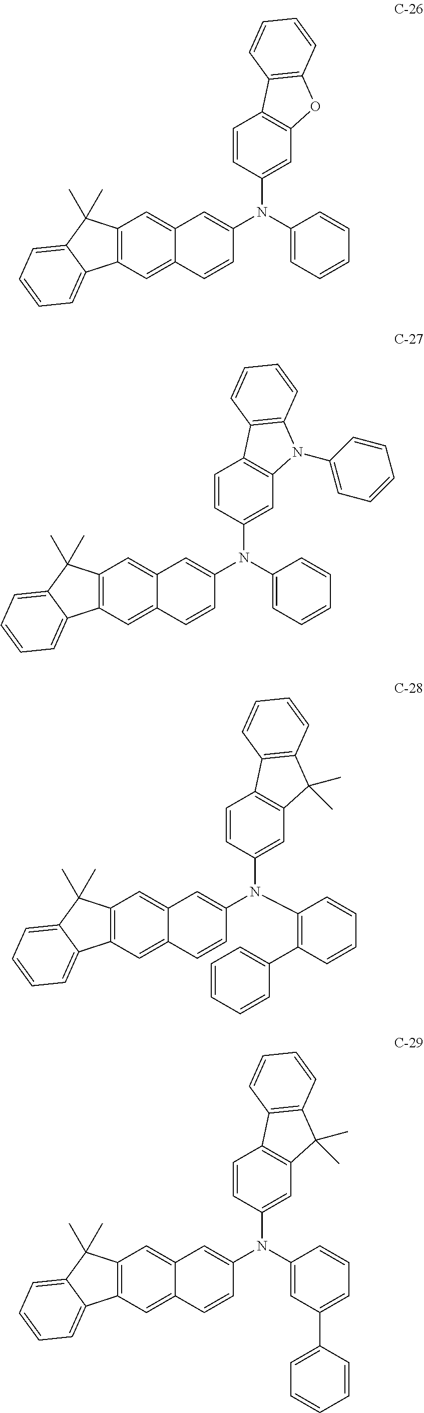 [SMILES: CC1(C)c2ccccc2-c2cc3ccc(N(c4ccccc4)c4ccc5c(c4)oc4ccccc45)cc3cc21 ; CC1(C)c2ccccc2-c2cc3ccc(N(c4ccccc4)c4ccc5c6ccccc6n(-c6ccccc6)c5c4)cc3cc21 ; CC1(C)c2ccccc2-c2ccc(N(c3ccc4cc5c(cc4c3)C(C)(C)c3ccccc3-5)c3ccccc3-c3ccccc3)cc21 ; CC1(C)c2ccccc2-c2ccc(N(c3cccc(-c4ccccc4)c3)c3ccc4cc5c(cc4c3)C(C)(C)c3ccccc3-5)cc21]

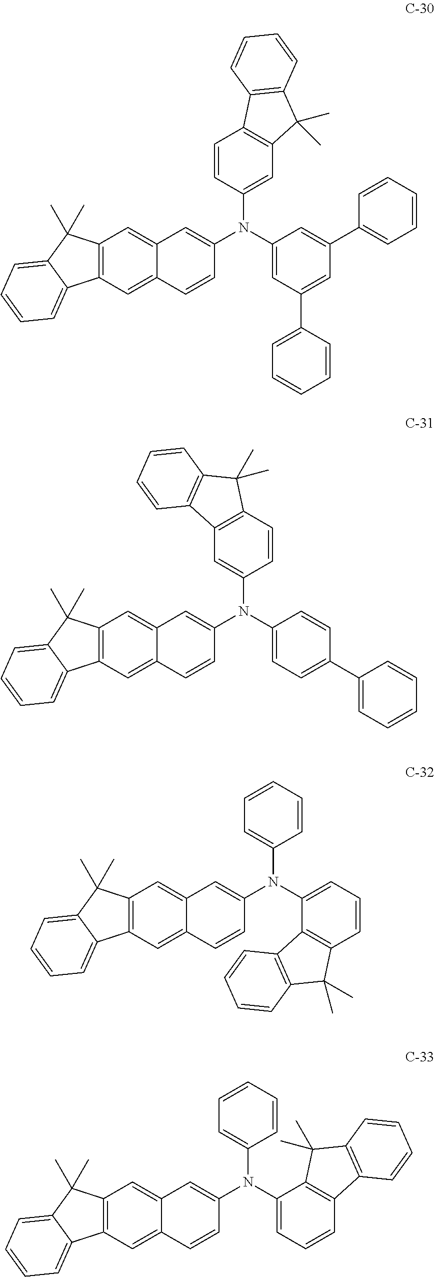 [SMILES: CC1(C)c2ccccc2-c2ccc(N(c3cc(-c4ccccc4)cc(-c4ccccc4)c3)c3ccc4cc5c(cc4c3)C(C)(C)c3ccccc3-5)cc21 ; CC1(C)c2ccccc2-c2cc(N(c3ccc(-c4ccccc4)cc3)c3ccc4cc5c(cc4c3)C(C)(C)c3ccccc3-5)ccc21 ; CC1(C)c2ccccc2-c2cc3ccc(N(c4ccccc4)c4cccc5c4-c4ccccc4C5(C)C)cc3cc21 ; CC1(C)c2ccccc2-c2cc3ccc(N(c4ccccc4)c4cccc5c4C(C)(C)c4ccccc4-5)cc3cc21]

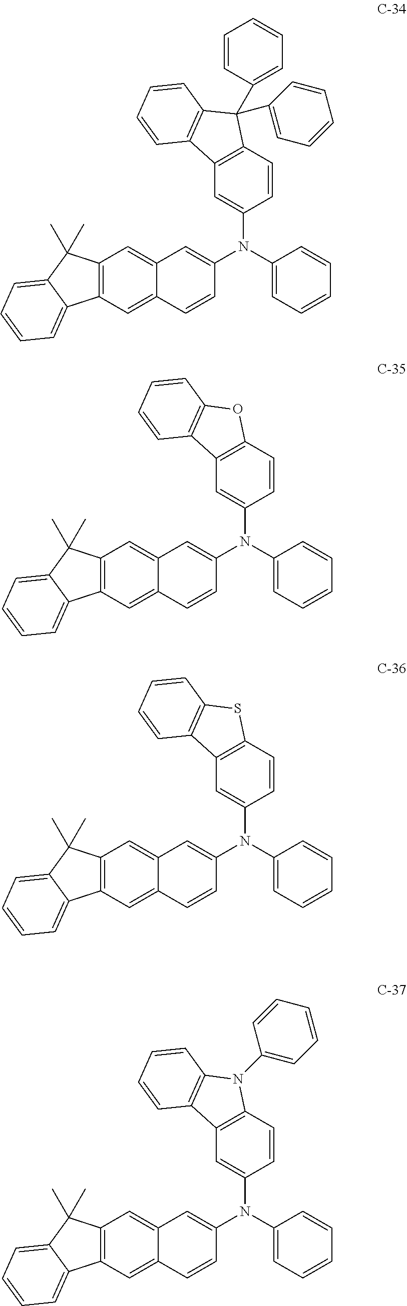 [SMILES: CC1(C)c2ccccc2-c2cc3ccc(N(c4ccccc4)c4ccc5c(c4)-c4ccccc4C5(c4ccccc4)c4ccccc4)cc3cc21 ; CC1(C)c2ccccc2-c2cc3ccc(N(c4ccccc4)c4ccc5oc6ccccc6c5c4)cc3cc21 ; CC1(C)c2ccccc2-c2cc3ccc(N(c4ccccc4)c4ccc5sc6ccccc6c5c4)cc3cc21 ; CC1(C)c2ccccc2-c2cc3ccc(N(c4ccccc4)c4ccc5c(c4)c4ccccc4n5-c4ccccc4)cc3cc21]

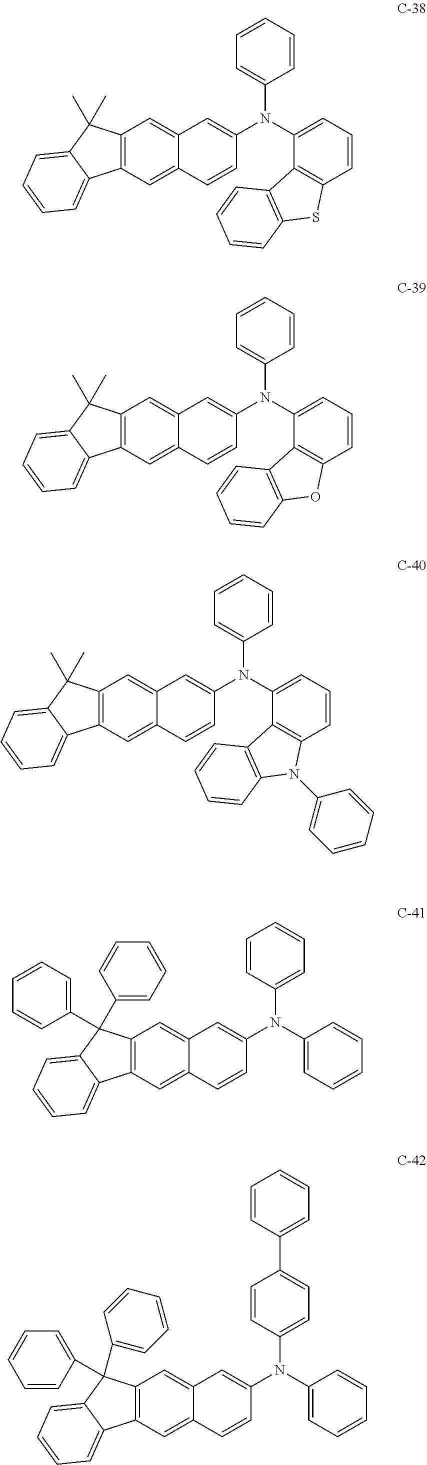 [SMILES: CC1(C)c2ccccc2-c2cc3ccc(N(c4ccccc4)c4cccc5sc6ccccc6c45)cc3cc21 ; CC1(C)c2ccccc2-c2cc3ccc(N(c4ccccc4)c4cccc5oc6ccccc6c45)cc3cc21 ; CC1(C)c2ccccc2-c2cc3ccc(N(c4ccccc4)c4cccc5c4c4ccccc4n5-c4ccccc4)cc3cc21 ; c1ccc(N(c2ccccc2)c2ccc3cc4c(cc3c2)C(c2ccccc2)(c2ccccc2)c2ccccc2-4)cc1 ; c1ccc(-c2ccc(N(c3ccccc3)c3ccc4cc5c(cc4c3)C(c3ccccc3)(c3ccccc3)c3ccccc3-5)cc2)cc1]

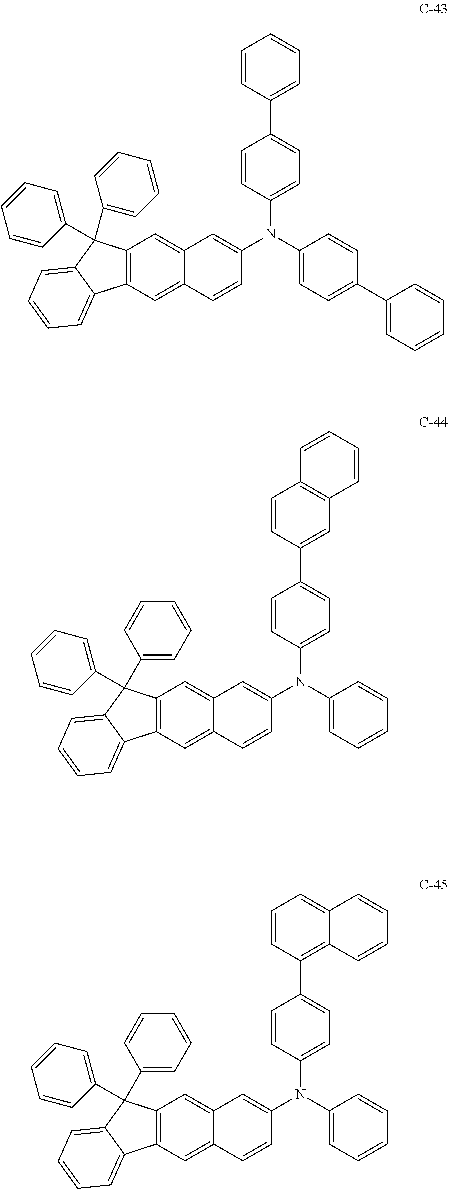 [SMILES: c1ccc(-c2ccc(N(c3ccc(-c4ccccc4)cc3)c3ccc4cc5c(cc4c3)C(c3ccccc3)(c3ccccc3)c3ccccc3-5)cc2)cc1 ; c1ccc(N(c2ccc(-c3ccc4ccccc4c3)cc2)c2ccc3cc4c(cc3c2)C(c2ccccc2)(c2ccccc2)c2ccccc2-4)cc1 ; c1ccc(N(c2ccc(-c3cccc4ccccc34)cc2)c2ccc3cc4c(cc3c2)C(c2ccccc2)(c2ccccc2)c2ccccc2-4)cc1]

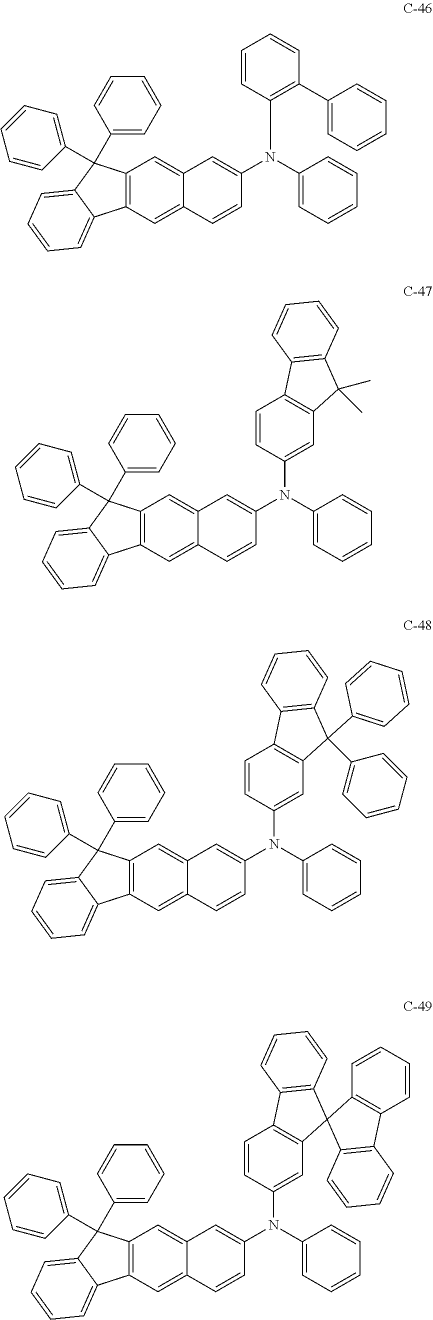 [SMILES: c1ccc(-c2ccccc2N(c2ccccc2)c2ccc3cc4c(cc3c2)C(c2ccccc2)(c2ccccc2)c2ccccc2-4)cc1 ; CC1(C)c2ccccc2-c2ccc(N(c3ccccc3)c3ccc4cc5c(cc4c3)C(c3ccccc3)(c3ccccc3)c3ccccc3-5)cc21 ; c1ccc(N(c2ccc3c(c2)C(c2ccccc2)(c2ccccc2)c2ccccc2-3)c2ccc3cc4c(cc3c2)C(c2ccccc2)(c2ccccc2)c2ccccc2-4)cc1 ; c1ccc(N(c2ccc3c(c2)C2(c4ccccc4-c4ccccc42)c2ccccc2-3)c2ccc3cc4c(cc3c2)C(c2ccccc2)(c2ccccc2)c2ccccc2-4)cc1]

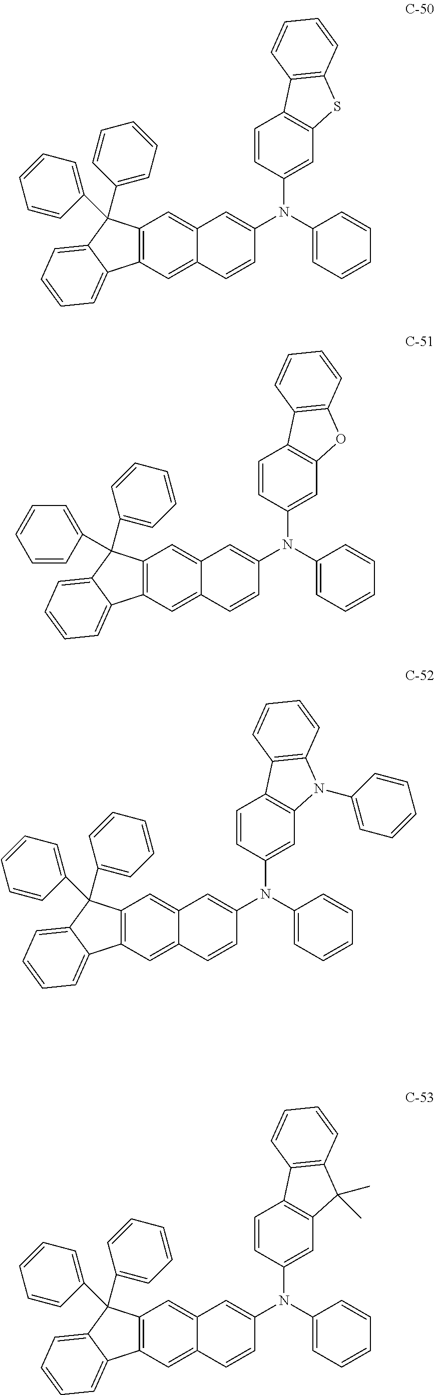 [SMILES: c1ccc(N(c2ccc3cc4c(cc3c2)C(c2ccccc2)(c2ccccc2)c2ccccc2-4)c2ccc3c(c2)sc2ccccc23)cc1 ; c1ccc(N(c2ccc3cc4c(cc3c2)C(c2ccccc2)(c2ccccc2)c2ccccc2-4)c2ccc3c(c2)oc2ccccc23)cc1 ; c1ccc(N(c2ccc3cc4c(cc3c2)C(c2ccccc2)(c2ccccc2)c2ccccc2-4)c2ccc3c4ccccc4n(-c4ccccc4)c3c2)cc1 ; CC1(C)c2ccccc2-c2ccc(N(c3ccccc3)c3ccc4cc5c(cc4c3)C(c3ccccc3)(c3ccccc3)c3ccccc3-5)cc21]

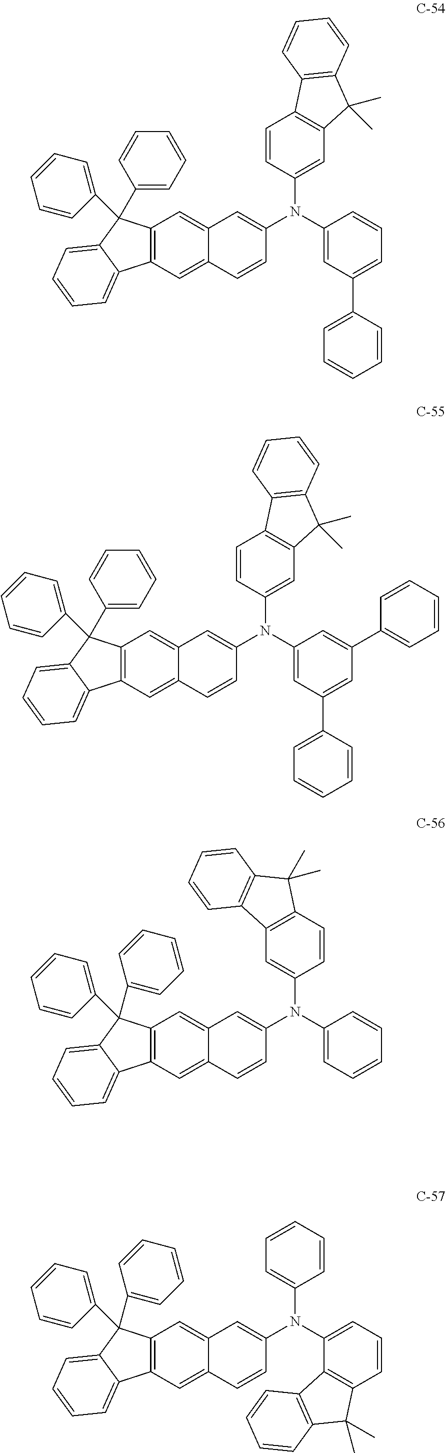 [SMILES: CC1(C)c2ccccc2-c2ccc(N(c3cccc(-c4ccccc4)c3)c3ccc4cc5c(cc4c3)C(c3ccccc3)(c3ccccc3)c3ccccc3-5)cc21 ; CC1(C)c2ccccc2-c2ccc(N(c3cc(-c4ccccc4)cc(-c4ccccc4)c3)c3ccc4cc5c(cc4c3)C(c3ccccc3)(c3ccccc3)c3ccccc3-5)cc21 ; CC1(C)c2ccccc2-c2cc(N(c3ccccc3)c3ccc4cc5c(cc4c3)C(c3ccccc3)(c3ccccc3)c3ccccc3-5)ccc21 ; CC1(C)c2ccccc2-c2c(N(c3ccccc3)c3ccc4cc5c(cc4c3)C(c3ccccc3)(c3ccccc3)c3ccccc3-5)cccc21]

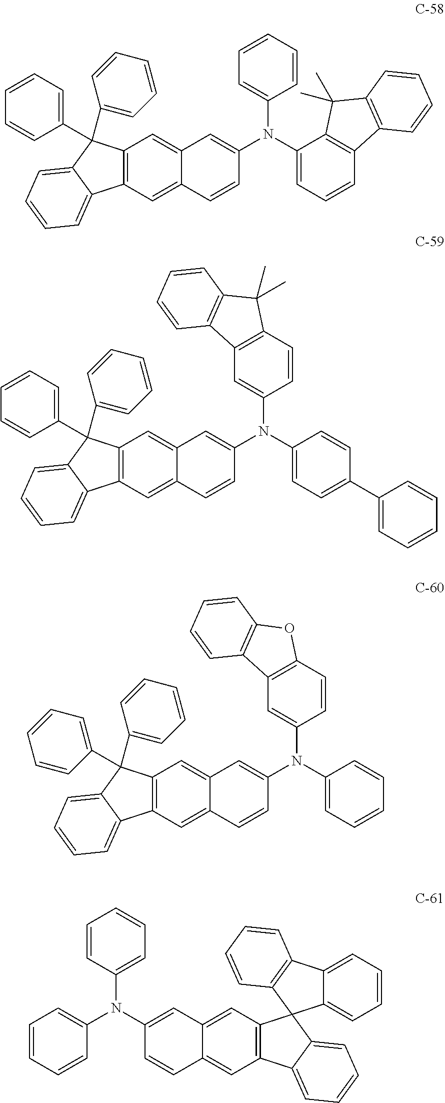 [SMILES: CC1(C)c2ccccc2-c2cccc(N(c3ccccc3)c3ccc4cc5c(cc4c3)C(c3ccccc3)(c3ccccc3)c3ccccc3-5)c21 ; CC1(C)c2ccccc2-c2cc(N(c3ccc(-c4ccccc4)cc3)c3ccc4cc5c(cc4c3)C(c3ccccc3)(c3ccccc3)c3ccccc3-5)ccc21 ; c1ccc(N(c2ccc3cc4c(cc3c2)C(c2ccccc2)(c2ccccc2)c2ccccc2-4)c2ccc3oc4ccccc4c3c2)cc1 ; c1ccc(N(c2ccccc2)c2ccc3cc4c(cc3c2)C2(c3ccccc3-c3ccccc32)c2ccccc2-4)cc1]

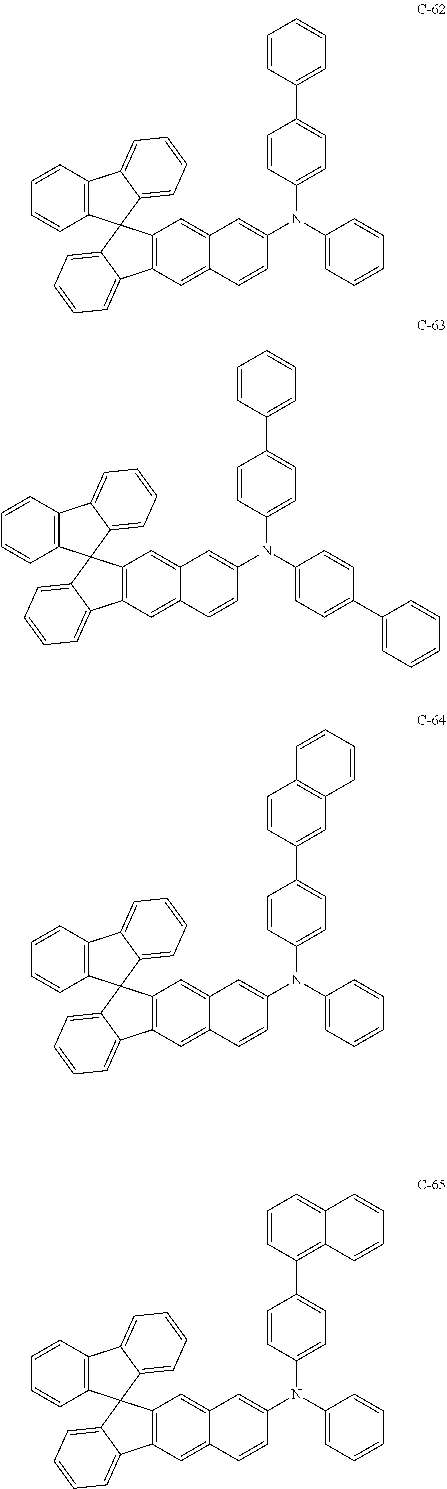 [SMILES: c1ccc(-c2ccc(N(c3ccccc3)c3ccc4cc5c(cc4c3)C3(c4ccccc4-c4ccccc43)c3ccccc3-5)cc2)cc1 ; c1ccc(-c2ccc(N(c3ccc(-c4ccccc4)cc3)c3ccc4cc5c(cc4c3)C3(c4ccccc4-c4ccccc43)c3ccccc3-5)cc2)cc1 ; c1ccc(N(c2ccc(-c3ccc4ccccc4c3)cc2)c2ccc3cc4c(cc3c2)C2(c3ccccc3-c3ccccc32)c2ccccc2-4)cc1 ; c1ccc(N(c2ccc(-c3cccc4ccccc34)cc2)c2ccc3cc4c(cc3c2)C2(c3ccccc3-c3ccccc32)c2ccccc2-4)cc1]

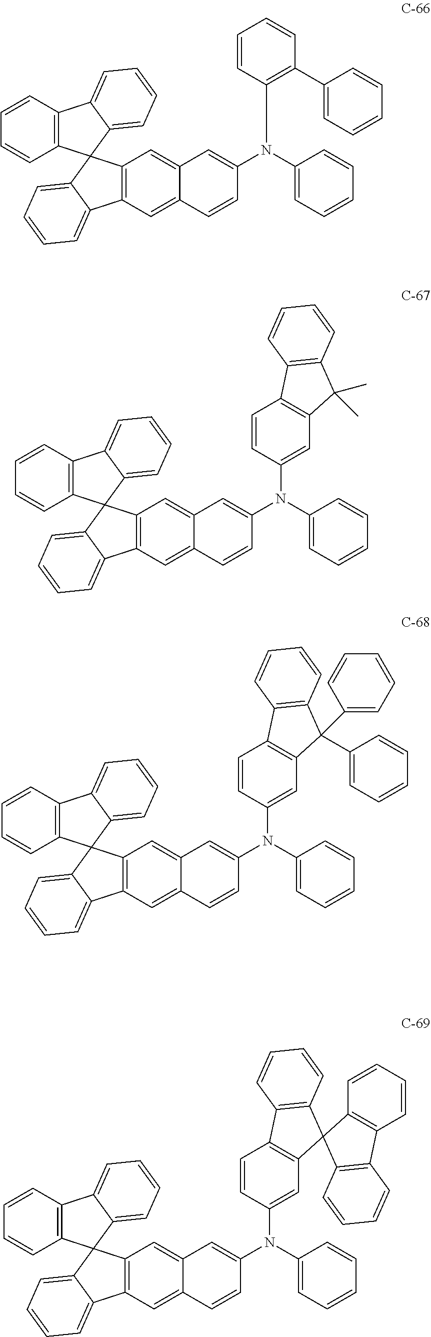 [SMILES: c1ccc(-c2ccccc2N(c2ccccc2)c2ccc3cc4c(cc3c2)C2(c3ccccc3-c3ccccc32)c2ccccc2-4)cc1 ; CC1(C)c2ccccc2-c2ccc(N(c3ccccc3)c3ccc4cc5c(cc4c3)C3(c4ccccc4-c4ccccc43)c3ccccc3-5)cc21 ; c1ccc(N(c2ccc3c(c2)C(c2ccccc2)(c2ccccc2)c2ccccc2-3)c2ccc3cc4c(cc3c2)C2(c3ccccc3-c3ccccc32)c2ccccc2-4)cc1 ; c1ccc(N(c2ccc3c(c2)C2(c4ccccc4-c4ccccc42)c2ccccc2-3)c2ccc3cc4c(cc3c2)C2(c3ccccc3-c3ccccc32)c2ccccc2-4)cc1]

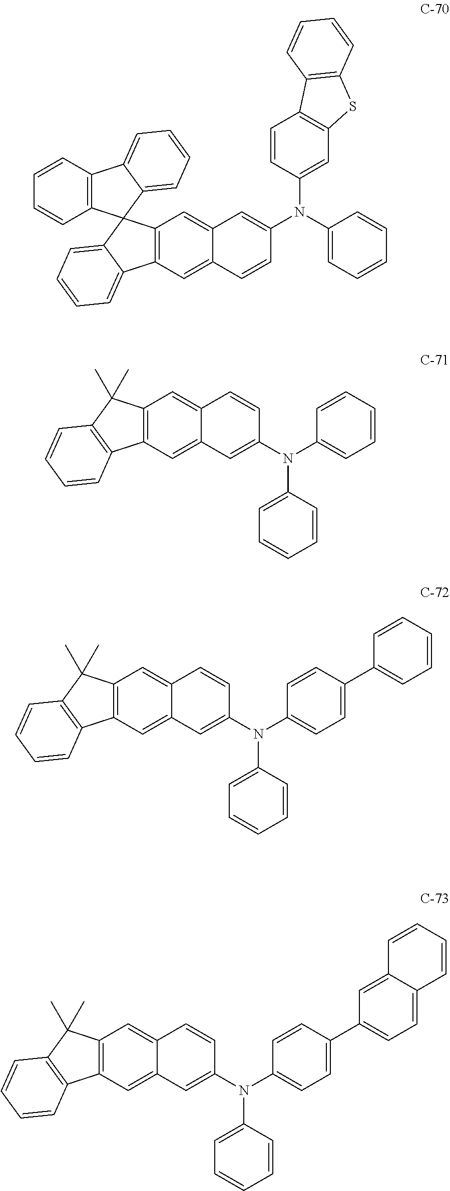 [SMILES: c1ccc(N(c2ccc3cc4c(cc3c2)C2(c3ccccc3-c3ccccc32)c2ccccc2-4)c2ccc3c(c2)sc2ccccc23)cc1 ; CC1(C)c2ccccc2-c2cc3cc(N(c4ccccc4)c4ccccc4)ccc3cc21 ; CC1(C)c2ccccc2-c2cc3cc(N(c4ccccc4)c4ccc(-c5ccccc5)cc4)ccc3cc21 ; CC1(C)c2ccccc2-c2cc3cc(N(c4ccccc4)c4ccc(-c5ccc6ccccc6c5)cc4)ccc3cc21]

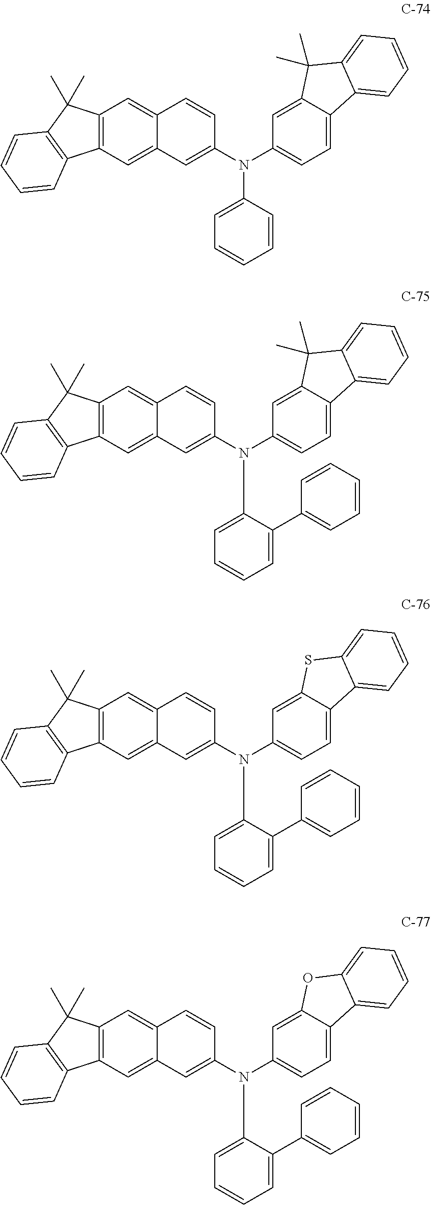 [SMILES: CC1(C)c2ccccc2-c2ccc(N(c3ccccc3)c3ccc4cc5c(cc4c3)-c3ccccc3C5(C)C)cc21 ; CC1(C)c2ccccc2-c2ccc(N(c3ccc4cc5c(cc4c3)-c3ccccc3C5(C)C)c3ccccc3-c3ccccc3)cc21 ; CC1(C)c2ccccc2-c2cc3cc(N(c4ccc5c(c4)sc4ccccc45)c4ccccc4-c4ccccc4)ccc3cc21 ; CC1(C)c2ccccc2-c2cc3cc(N(c4ccc5c(c4)oc4ccccc45)c4ccccc4-c4ccccc4)ccc3cc21]

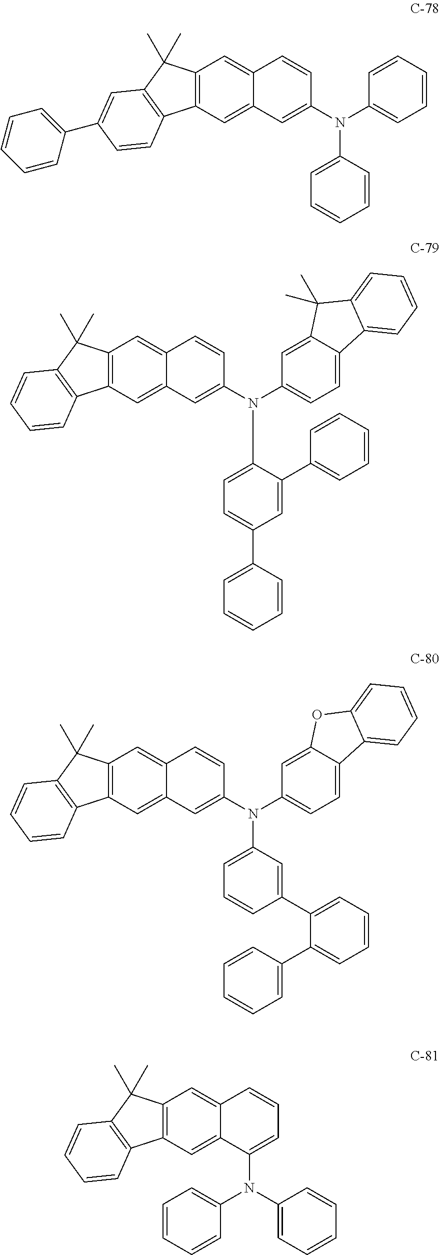 [SMILES: CC1(C)c2cc(-c3ccccc3)ccc2-c2cc3cc(N(c4ccccc4)c4ccccc4)ccc3cc21 ; CC1(C)c2ccccc2-c2ccc(N(c3ccc4cc5c(cc4c3)-c3ccccc3C5(C)C)c3ccc(-c4ccccc4)cc3-c3ccccc3)cc21 ; CC1(C)c2ccccc2-c2cc3cc(N(c4cccc(-c5ccccc5-c5ccccc5)c4)c4ccc5c(c4)oc4ccccc45)ccc3cc21 ; CC1(C)c2ccccc2-c2cc3c(N(c4ccccc4)c4ccccc4)cccc3cc21]

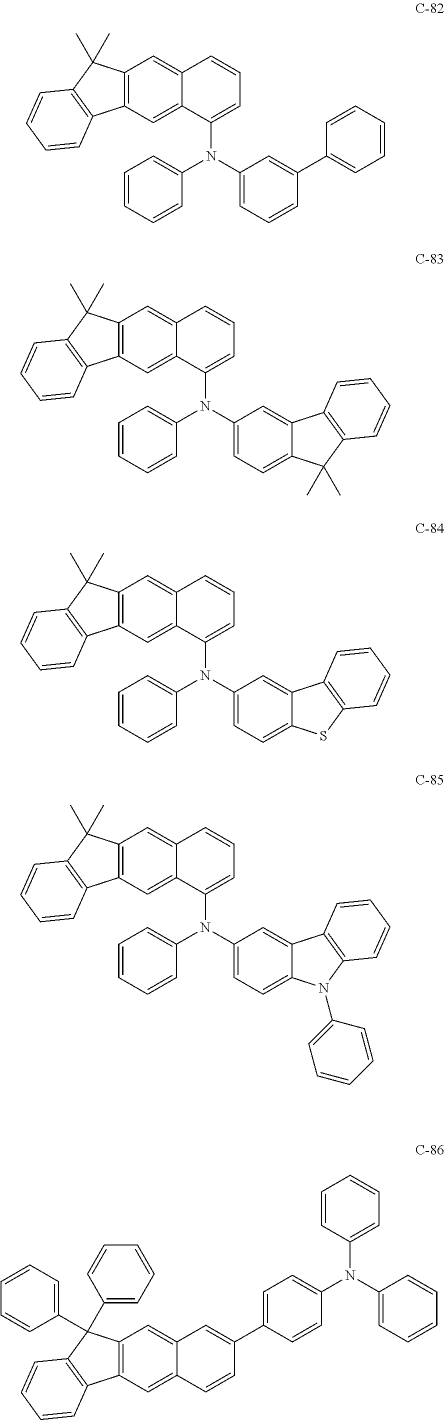 [SMILES: CC1(C)c2ccccc2-c2cc3c(N(c4ccccc4)c4cccc(-c5ccccc5)c4)cccc3cc21 ; CC1(C)c2ccccc2-c2cc(N(c3ccccc3)c3cccc4cc5c(cc34)-c3ccccc3C5(C)C)ccc21 ; CC1(C)c2ccccc2-c2cc3c(N(c4ccccc4)c4ccc5sc6ccccc6c5c4)cccc3cc21 ; CC1(C)c2ccccc2-c2cc3c(N(c4ccccc4)c4ccc5c(c4)c4ccccc4n5-c4ccccc4)cccc3cc21 ; c1ccc(N(c2ccccc2)c2ccc(-c3ccc4cc5c(cc4c3)C(c3ccccc3)(c3ccccc3)c3ccccc3-5)cc2)cc1]

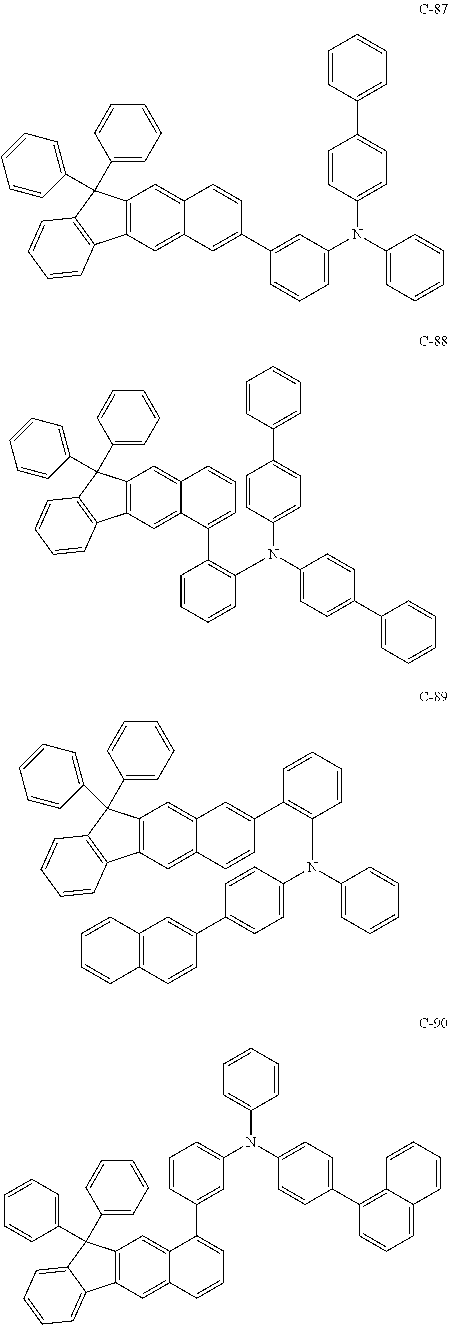 [SMILES: c1ccc(-c2ccc(N(c3ccccc3)c3cccc(-c4ccc5cc6c(cc5c4)-c4ccccc4C6(c4ccccc4)c4ccccc4)c3)cc2)cc1 ; c1ccc(-c2ccc(N(c3ccc(-c4ccccc4)cc3)c3ccccc3-c3cccc4cc5c(cc34)-c3ccccc3C5(c3ccccc3)c3ccccc3)cc2)cc1 ; c1ccc(N(c2ccc(-c3ccc4ccccc4c3)cc2)c2ccccc2-c2ccc3cc4c(cc3c2)C(c2ccccc2)(c2ccccc2)c2ccccc2-4)cc1 ; c1ccc(N(c2ccc(-c3cccc4ccccc34)cc2)c2cccc(-c3cccc4cc5c(cc34)C(c3ccccc3)(c3ccccc3)c3ccccc3-5)c2)cc1]

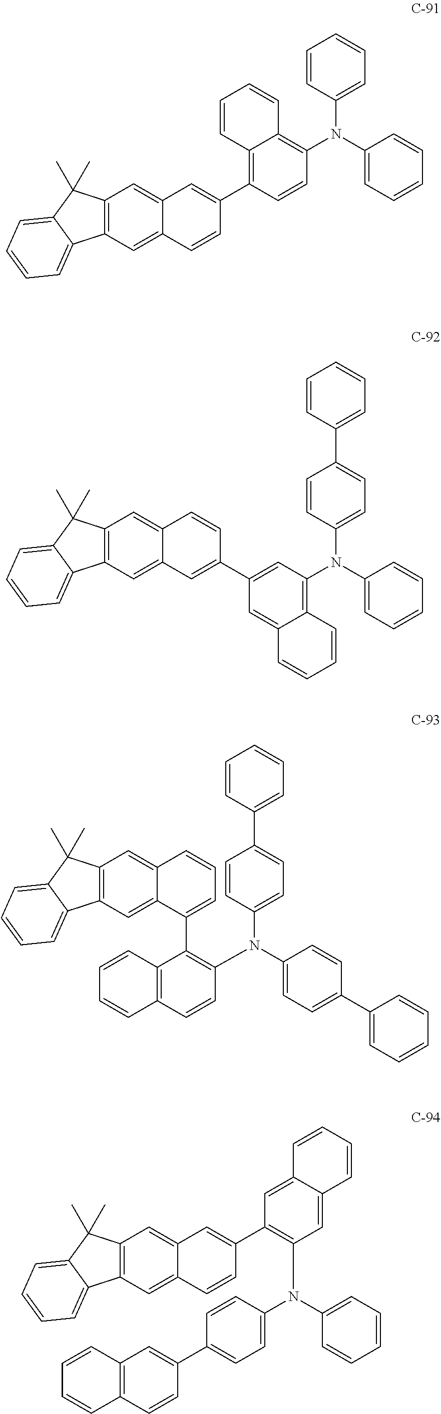 [SMILES: CC1(C)c2ccccc2-c2cc3ccc(-c4ccc(N(c5ccccc5)c5ccccc5)c5ccccc45)cc3cc21 ; CC1(C)c2ccccc2-c2cc3cc(-c4cc(N(c5ccccc5)c5ccc(-c6ccccc6)cc5)c5ccccc5c4)ccc3cc21 ; CC1(C)c2ccccc2-c2cc3c(-c4c(N(c5ccc(-c6ccccc6)cc5)c5ccc(-c6ccccc6)cc5)ccc5ccccc45)cccc3cc21 ; CC1(C)c2ccccc2-c2cc3ccc(-c4cc5ccccc5cc4N(c4ccccc4)c4ccc(-c5ccc6ccccc6c5)cc4)cc3cc21]

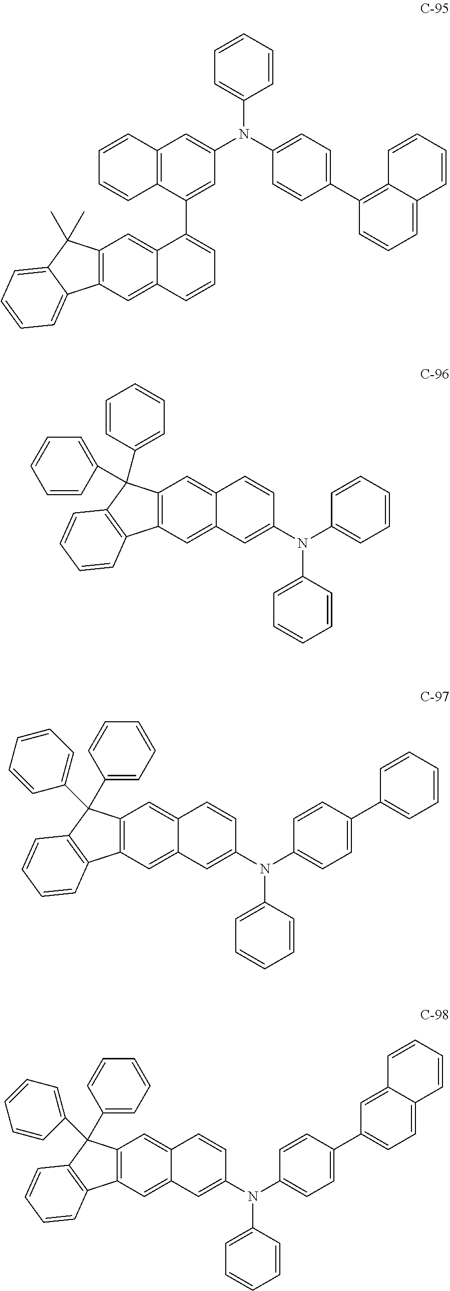 [SMILES: CC1(C)c2ccccc2-c2cc3cccc(-c4cc(N(c5ccccc5)c5ccc(-c6cccc7ccccc67)cc5)cc5ccccc45)c3cc21 ; c1ccc(N(c2ccccc2)c2ccc3cc4c(cc3c2)-c2ccccc2C4(c2ccccc2)c2ccccc2)cc1 ; c1ccc(-c2ccc(N(c3ccccc3)c3ccc4cc5c(cc4c3)-c3ccccc3C5(c3ccccc3)c3ccccc3)cc2)cc1 ; c1ccc(N(c2ccc(-c3ccc4ccccc4c3)cc2)c2ccc3cc4c(cc3c2)-c2ccccc2C4(c2ccccc2)c2ccccc2)cc1]

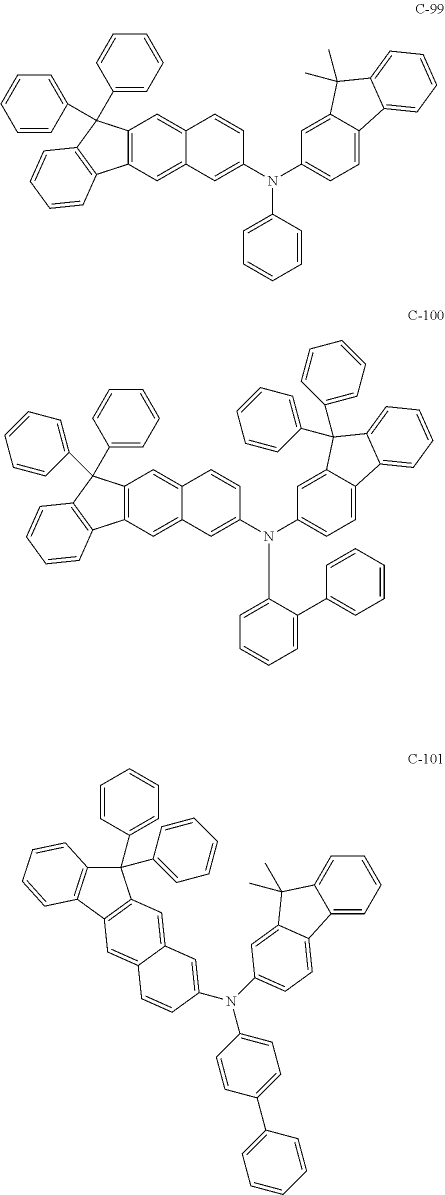 [SMILES: CC1(C)c2ccccc2-c2ccc(N(c3ccccc3)c3ccc4cc5c(cc4c3)-c3ccccc3C5(c3ccccc3)c3ccccc3)cc21 ; c1ccc(-c2ccccc2N(c2ccc3c(c2)C(c2ccccc2)(c2ccccc2)c2ccccc2-3)c2ccc3cc4c(cc3c2)-c2ccccc2C4(c2ccccc2)c2ccccc2)cc1 ; CC1(C)c2ccccc2-c2ccc(N(c3ccc(-c4ccccc4)cc3)c3ccc4cc5c(cc4c3)C(c3ccccc3)(c3ccccc3)c3ccccc3-5)cc21]

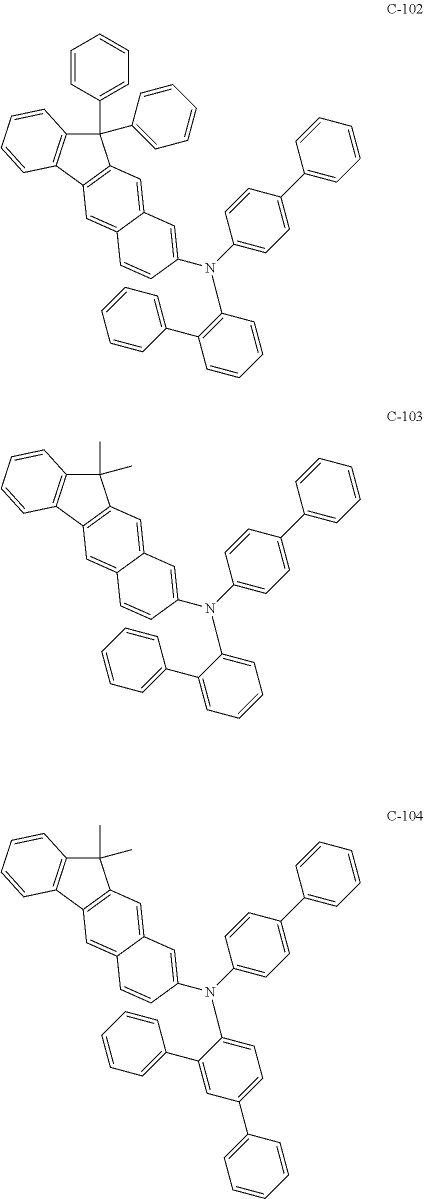 [SMILES: c1ccc(-c2ccc(N(c3ccc4cc5c(cc4c3)C(c3ccccc3)(c3ccccc3)c3ccccc3-5)c3ccccc3-c3ccccc3)cc2)cc1 ; CC1(C)c2ccccc2-c2cc3ccc(N(c4ccc(-c5ccccc5)cc4)c4ccccc4-c4ccccc4)cc3cc21 ; CC1(C)c2ccccc2-c2cc3ccc(N(c4ccc(-c5ccccc5)cc4)c4ccc(-c5ccccc5)cc4-c4ccccc4)cc3cc21]

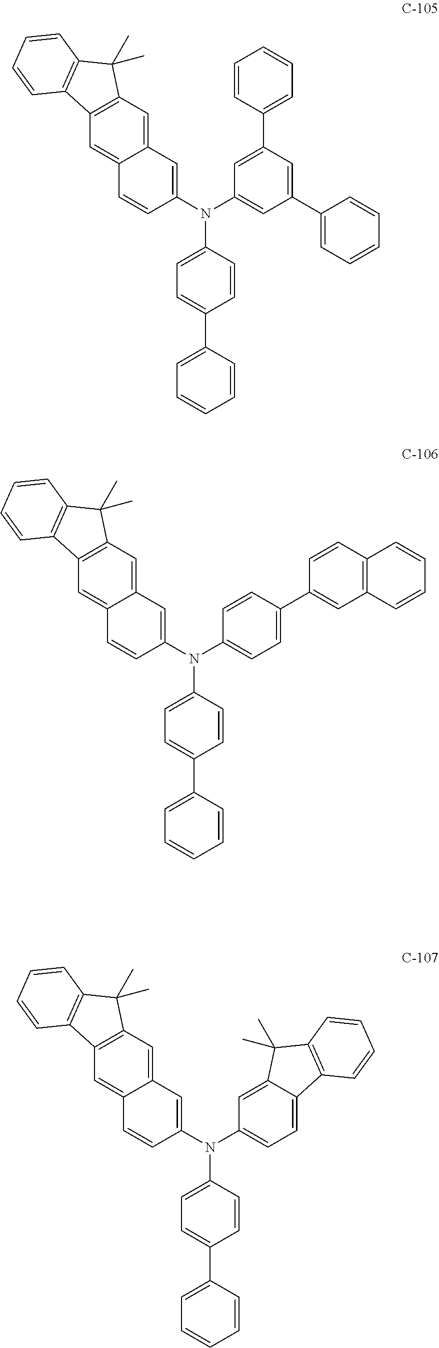 [SMILES: CC1(C)c2ccccc2-c2cc3ccc(N(c4ccc(-c5ccccc5)cc4)c4cc(-c5ccccc5)cc(-c5ccccc5)c4)cc3cc21 ; CC1(C)c2ccccc2-c2cc3ccc(N(c4ccc(-c5ccccc5)cc4)c4ccc(-c5ccc6ccccc6c5)cc4)cc3cc21 ; CC1(C)c2ccccc2-c2ccc(N(c3ccc(-c4ccccc4)cc3)c3ccc4cc5c(cc4c3)C(C)(C)c3ccccc3-5)cc21]

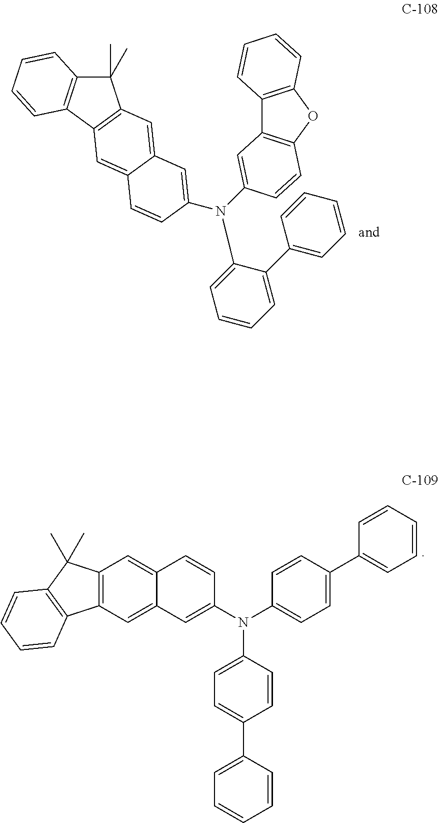 [SMILES: CC1(C)c2ccccc2-c2cc3ccc(N(c4ccc5oc6ccccc6c5c4)c4ccccc4-c4ccccc4)cc3cc21 ; CC1(C)c2ccccc2-c2cc3cc(N(c4ccc(-c5ccccc5)cc4)c4ccc(-c5ccccc5)cc4)ccc3cc21]

5. An organic electroluminescent material comprising the organic electroluminescent compound according to claim 1.

6. The organic electroluminescent material according to claim 5, wherein the organic electroluminescent material is the material of a hole transport zone.

7. The organic electroluminescent material according to claim 5, wherein the material of a hole transport zone is at least one of a hole injection material, a hole transport material, a hole auxiliary material, a light-emitting auxiliary material, and an electron blocking material.

8. An organic electroluminescent device comprising the organic electroluminescent compound according to claim 1.

9. The organic electroluminescent device according to claim 8, wherein the organic electroluminescent compound is comprised in the layer of a hole transport zone.
